SEQUENCE LISTING (29 sequences)

SEQ ID NO: 1 - protein, 724 aa, Thermoascus aurantiacus
ACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAE

SEQ ID NO: 2 - protein, 717 aa, Mycothermus thermophilus
MNRVTNLLAWAGAIGLAQATCPFADPAALYSRQDTTSGQSPLAAYEVDDSTGYLTSDVGGPIQDQTSLKAGIRGPTLLEDFMFRQKIQHFDHERVPERAVHARGAGAHGTFTSYADWSNITAASFLNATGKQTPVFVRFSTVAGSRGSADTARDVHGFATRFYTDEGNFDIVGNNIPVFFIQDAIQFPDLIHSVKPRPDNEIPQAATAHDSAWDFFSQQPSTMHTLFWAMSGHGIPRSYRHMDGFGVHTFRFVKDDGSSKLIKWHFKSRQGKASLVWEEAQVLSGKNADFHRQDLWDAIESGNGPEWDVCVQIVDESQAQAFGFDLLDPTKIIPEEYAPLTKLGLLKLDRNPTNYFAETEQVMFQPGHIVRGIDFTEDPLLQGRLFSYLDTQLNRNGGPNFEQLPINMPRVPIHNNNRDGAGQMFIHRNKYPYTPNTLNSGYPRQANQNAGRGFFTAPGRTASGALVREVSPTFNDHWSQPRLFFNSLTPVEQQFLVNAMRFEISLVKSEEVKKNVLTQLNRVSHDVAVRVAAAIGLGAPDADDTYYHNNKTAGVSIVGSGPLPTIKTLRVGILATTSESSALDQAAQLRTRLEKDGLVVTVVAETLREGVDQTYSTADATGFDGVVVVDGAAALFASTASSPLFPTGRPLQIFVDAYRWGKPVGVCGGKSSEVLDAADVPEDGDGVYSEESVDMFVEEFEKGLATFRFTDRFALDS

SEQ ID NO: 3 - protein, 351 aa, Coprinus cinereus
MVSCKLPLPLLTLAIALANVNAFPAYQSLGGLSKRQLETIIPGLPVVNPGPPPGPLADSTLKLVNDAAHPYQAPRPHLDHRGPCPGLNTLANHGYLPRSGIATPAQIVQAVMEGFNMENTFAKFVTYAAFLVDGNPITNLMSIGGKTWRTGIIEPPPPAIVGGLNTHAVFEGDTSMTRGDFHFGDNHSFNQTLFDQFVEYSNIHGGGFYNLTAATELRYQRIQQSIATNPEMSFVSPRWFTAILLQDEKFPDDFHRAPGPFSFEGLGYLVTRRPMPPGRNVGGVDNYVPDPNSADFNSFCKMYEDFVNDIVVALYPNPTGLLRRNLIKNLEYFWTGMFDPACTEVKPYGTL

SEQ ID NO: 4 - protein, 598 aa, Talaromyces emersonii
RAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 5 - protein, 556 aa, Gloeophyllum sepiarum
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 6 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 7 - protein, 573 aa, Pycnoporus sanguineus
MRFTLLASLIGLAVGAFAQSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 8 - protein, 574 aa, Triametes cinglulata
MRFTLLTSLLGLALGAFAQSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSSTVNLPASTTIEYKYIRKFNGAVTWESDPNNSITTPASGTFTQNDTWR

SEQ ID NO: 9 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 10 - protein, 515 aa, Bacillus thermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 11 - protein, 356 aa, Dictyogllomus thermophilum
MKKPLGKIVASTALLISVAFSSSIASAKEEAKGMEIPSLKEVYKDYFTIGAAVSHLNIYHYENLLKKHFNSLTPENQMKWEVIHPKPYVYDFGPADEIVDFAMKNGMKVRGHTLVWHNQTPGWVYAGTKDEILARLKEHIKEVVGHYKGKVYAWDVVNEALSDNPNEFLRRAPWYDICGEEVIEKAFIWAHEVDPDAKLFYNDYNLEDPIKREKAYKLVKKLKDKGVPIHGIGIQGHWTLAWPTPKMLEDSIKRFAELGVEVQVTEFDISIYYDRNENNNFKVPPEDRLERQAQLYKEAFEILRKYKGIVTGVTFWGVADDYTWLYFWPVRGREDYPLLFDKNHNPKKAFWEIVKF

SEQ ID NO: 12 - protein, 203 aa, Dictyogllomus thermophilum
QTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGS

SEQ ID NO: 13 - protein, 383 aa, Rasomsonia byssochlamydoides
DGLNTAAKAIGKLYFGTATDNPELSDVAYETQLNNTQDFGQITPANSMKWDATEPEQNTFTFAAGDQIADLAEANGQILRCHNLVWYNQLPSWVTSGSWTNETLLAAMKNHITNVVTHYKGRCYAWDVVNEALNDDGTYRDNVFYQYIGEAYIPIAFETAAAADPNVKLYYNDYNIEYAGVKATAAQNIVKLVQSYGARIDGVGLQSHFIVGETPSTSTQASNMASFTALGVEVAITELDIRMQLPETTALLTQQSTDYQSTVQACVNTPGCVGITLWDWTDKYSWVPSTFSGYGDACPWDDNYQKKPAYYGILTALGGSASTTTVGTGTTTTSTATTSSTGSSGTGVAQHWGQCGGIGWTGPTVCASGYTCTVVNPYYSQCL

SEQ ID NO: 14 - protein, 385 aa, Talaromyces leycettanus
AGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 15 - protein, 378 aa, Aspergillus fumigatus
AGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 16 - protein, 409 aa, Talaromyces leycettanus
MKFSNVILAASASSLVLAAPKSKTKRTSAFQWFGANESGAEFGNQNIPGTLGTDYTWPDTSTIQTLRNAGMNIFRVPFLMERLVPNQMTGSPDPTYLADLKSTVNFITGTGAYAVVDPHNYGRYYNNIITSTSDFAAFWTTVASQFASNPRVIFDTNNEYNNMDQTLVLNLNQAAINAIRAAGATSQYIFAEGNSWTGAWTWTSVNDNMKQLTDPSNKLVYEMHQYLDSDGSGTSDQCVNSTIGYDRIVSATQWLQANGKVAFLGEFAGGSNSVCEAAVTGMLDYMEQNSDVWLGAEWWAAGPWWGNYIYSMEPPSGIAYQNYLSILEPYFPGGSYSGGTGSGSGSTTTTATTTTTKVPPTSTTSSASSTGTGVAQHWGQCGGQGWTGPTTCVSPYTCQELNPYYYQCL

SEQ ID NO: 17 - protein, 334 aa, Penicillium capsulatum
MKFSNLVALAAAASSAMALPLTKKHAKRASSFEWFGSNESGAEFGSGNIPGVYGTDYIFPSTSAIQTLINNGMNIFRVTFMMERLVPNTMTGSFDAEYLSNLTSVVNYITEAGAHAVIDPHNYGRYYGSIISSTSDFQTFWKNVAGQFKSNSLVIFDTNNEYHDMDQTLVLNLNQAAINGIRAAGATSQYIFVEGNSYTGAWTWADVNDNLKNLTDPQNKIVYEMHQYLDSDGSGTSATCVSTTIGKERVTSATQWLQKNGKVGILGEFAGGVNDQCKTAITGMLSYLEDNSDVWRGAMWWAAGPWWGDYIFSLEPPSGTAYTGMWSTLKSYLA

SEQ ID NO: 18 - protein, 394 aa, Trichophaea saccata
MHSFFSLALAVAGLPALINAQQSAWGQCGGVGWTGATTCVSGYYCSKLNDYYSQCIPGTASTTTSAVSTTTTATSPTGSVCSGNRTKFKYFGVNESGAEFGNNVVPGTLGKDYTWPTTDSVDFFLGKGMNTFRIAFLMERLSPPAGGLTGTFDPTYLASLKNIASYITGKGGYAIIDPHNYGRYNGNIITDYTSFGTWCKNLASQFKSDSHIIFDTNNEYHDMDETLVFNLNQACINGIRAAGATSQLILIEGNSWTGAWTWISSGNAASLINLTDPNNNIAYEMHQYLDSDGSGTSPTCVSSTIGAERLAAATAWLQANNKKGFLGEIGAGSNDDCIAAVKGALCSMQEAGGVWLGALWWAAGPWWGDYYQSIEPPDGAAIARILPEALLPFL

SEQ ID NO: 19 - protein, 294 aa, Sordaria fimicola
MRSSTILQTGLVAVLPFAVQAASGSGKSTRYWDCCKPSCAWSGKASVNRPVLACDANNNPLNDANVKSGCDGGSAYTCANNSPWAVNDNLAYGFAATKLSGGTESSWCCACYALTFTSGPVSGKTLVVQSTSTGGDLGSNHFDLNMPGGGVGLFDGCKREFGGLPGAQYGGISSRSECDSFPAALKPGCQWRFDWFKNADNPEFTFKQVQCPSELTSRTGCKRNDDSQFPAFTPPSGGGSNPSTPTTPPSSGGGGSGCAAAMYAQCGGSGFSGCTNCPSGSTCKAINDYYHQCA

SEQ ID NO: 20 - protein, 278 aa, Thielavia terrestris [sequence omitted]

SEQ ID NO: 21 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 22 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 23 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 24 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 25 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 26 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 27 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 28 - protein, 1038 aa, Talaromyces funiculosus
MYSATLLQALCLLPLAAGLPFNERVDQVLRSYEVTSKLDSRSTKPSKHGHTYQTQFLGVTWDQRNWRLQSTVLDQGHYESRGSIANGYIGLNVAGAGPLFELDSPVDGDVINGWPLFSRRQTFAGLAGFYDLQPRTNGTNFPWLSQYGDDSAISGVPHWGGMVLDLGDGEYLDATVDNSTISDYTTTYDYKAGVLSWDYKWTPKNANGSFGISYKIFANKLDVNQAVVQLSITPSTNGSASVVNVIDGYAAVRTDFVSSGNESDVVYTAVKPNGVTNVTAWIYTALDGDDAFDISSAALVNDKPYVHQNDSSIAQSVNVTFTAGTTITINKFVGAASTDAFPDPQSTAREAALSARRRGFDDLFRSHISEWAQVMPDDSVDDFTLANGTLPNDTFIIESAVMAVVNPYYLLQNTVGPNALRRVNNAPVNDWSIPVGGLTSDSYAGQIFWDADVWMQPGLVAAFPESAKRITNYRAAKYSQALENAKTAYTSSQNQTWFSPDAAIYSWTSGRVGNCTATGPCWDYEYHLNGDIGISLVNEWVVSGDNETFKNKHFPIYNSIATLYGDLLKKNGSYYTLTNMTDPDEYANNVDAGGYTMTLISQTLSNANAFRKQFGMNENTTWTEMADNILLIRENDVTLEYTTMNNSVAVKQADVILSTFPLDYTKNYTTSAALNDLDYYALKQSPDGPGMTYAIFSIVANDVSPSGCSAYTYAQYSYDPYIRGPFFQFSEQLLDDYTINGGTHPAFPFLTGHGGANQVVLYGYLGLRLLPDDMLHIDPNLPPQIPSIKYRTFYWRGWPIQAASNYTHTTIQRATTVAPLSTADPTYANKSIHVSVGHNTVNSTTYSLSANGSALVVPNRQIGSINTVAGNVVQCKSVLSTDAYQKGQYPISAVDGAASTKWQPEFAANISSLTVDLTGSNVSSVSGFYFDWAQAPPTNITVLLHNSSSAALASSGDKPGSSAVTLNITISNPYNASTYNANIIALPSSNSTNYTFPAPVPKPRYATLFVQGNQALDETDTKSGNGTGATVAEWAILS

SEQ ID NO: 29 - protein, 1089 aa, Talaromyces leycettanus
MQSKLLALLPLLLQLPAVSGTSANARINRCVKKHAGGKTPSGPSNNTYQTRFPGVTWDQDNWCLSTTTLDQGHYESRGSVANGYLGINVASVGPFFEFDTPVDGDVINGWPLFDRRMSFATISGFWDQQPTTNGSNFPWLYQYGGESVISGVPHWSGLILDLGDNTYLDATVDSRTISGFSTTYDFKSGVLSWSYQWTPAGNMGSYNITYRLFAHKLYVNQAVVDMEVVSSTEAKATVVNVIDGASAVRTDFVESGQDDGAIYTAVRPWGIANVTAYIYANITGSDNVDMRSRALVTNKPYVNGNASSITQAVNVHFTPGKSVRITKFVGGASSDAFSNPQQIAKQACSTAQANGYVKSLRSHVAEWASVMPDDSVDDFTFPSNGTLPADEYIIESQIISVANTYYLLQNTVGKNAINASSSTELNKDSIAVGGLTSESYAGMIFWDADVWMQPGLVASHPEAAQRITNYRVAKYPQAKANVATAYQSSKNQTNFSPDAAVYSWTSARYGNCTATGPCWDYEYHLNGDIGLSIINQYVASGDTQTFKEKLFPVFDSVATLYSNIVQKNGSSWTLTNMTDPDEYANQVDAGGYTMPLIAQTLLYANSFRQQFGLETNDTWNEIAQDVLVIRENGVTLEFTTMNGSAVVKQADVVLDTYPLGYTHNYGPTDALNDLDYYANRQSPDGPAMTWAIFSVVANQISPSGCSAYTYAQYAFSPYARAPFYQLSEQLIDDASLNGGTHPAYPFLTGHGGALQVNLFGYLGFRYLPDNVIHIDPNLPPQIPHITYRTFYWRGWPITAASTYTHTTLSRAWNVSSLDSADPKFANASIPVHVGLESNVTVYRLPVNGTLTVPNRMVGSKNTLAGNMVQCRPVQSMDGYQPGQFPISVVDGASSTKWQPLYSANVSSVTVTLSSSAVGKSVNGFYFDWAQNPPVNAAVVFHNSSFAQNPATTFSFDNPSASGNLYSVVSVLKDIQLSDPYDPATTDLDVIAIPKGNTTNVTLSSPVPAARYATLFIQGNQANSPAEVAAKNGTGATVAEWAILGQEVQNNGYGDQIEARRLDVRGAAALSGMGSFTQRRKRKMILPRFD

(19) United States
(12) Patent Application Publication
(10) Pub. No.: US 2021/0198618 A1
(43) Pub. Date: Jul. 1, 2021

(54) PROCESSES FOR ENHANCING YEAST GROWTH AND PRODUCTIVITY

(71) Applicant: NOVOZYMES A/S, Bagsvaerd (DK)

(72) Inventors: Angela Shows, Franklinton, NC (US); Armindo Ribeiro Gaspar, Rolesville, NC (US)

(73) Assignee: NOVOZYMES A/S, Bagsvaerd (DK)

(21) Appl. No.: 17/056,823

(22) PCT Filed: May 29, 2019
(86) PCT No.: PCT/US2019/034241

§ 371 Date: Nov. 19, 2020

Related U.S. Application Data
(60) Provisional application No. 62/678,577, filed on May 31, 2018.

(51) Int. Cl. C12N 1/16; C12P 19/14; C12P 7/56; C12P 7/20; C12P 7/54; C12P 7/14

(52) U.S. Cl. CPC C12N 1/16; C12P 19/14; C12P 7/56; C12N 2501/71; C12P 7/54; C12P 7/14; C12P 7/20

(57) ABSTRACT

The invention relates to processes for enhancing yeast growth and/or productivity using peroxidase or a composition comprising peroxidase.

### REFERENCE TO A SEQUENCE LISTING

This application contains a Sequence Listing in computer readable form, which is incorporated herein by reference.

### FIELD OF THE INVENTION

The present invention relates to processes for enhancing yeast growth and/or productivity, for example during production of yeast and/or during yeast propagation, by contacting yeast with an effective amount of a peroxidase or peroxidase composition. The present invention also relates to processes for producing fermentation products, such as especially ethanol, wherein a peroxidase or peroxidase composition is used to accelerate yeasth growth and increase ethanol titers early in the fermentation process, and reduce lactic acid titers.

### BACKGROUND OF THE INVENTION

Fermentation products, such as ethanol, are typically produced by first grinding starch-containing material in a dry-grind or wet-milling process, then degrading the material into fermentable sugars using enzymes and finally converting the sugars directly or indirectly into the desired fermentation product using a fermenting organism. Liquid fermentation products are recovered from the fermented mash (often referred to as “beer mash”), e.g., by distillation, which separates the desired fermentation product, e.g. ethanol, from other liquids and/or solids. The remaining fraction is referred to as “whole stillage”. Whole stillage typically contains about 10 to 20% solids. The whole stillage is separated into a solid and a liquid fraction, e.g., by centrifugation. The separated solid fraction is referred to as “wet cake” (or “wet grains”) and the separated liquid fraction is referred to as “thin stillage”. Wet cake and thin stillage contain about 35 and 7% solids, respectively. Wet cake, with optional additional dewatering, is used as a component in animal feed or is dried to provide “Distillers Dried Grains” (DDG) used as a component in animal feed. Thin stillage is typically evaporated to provide evaporator condensate and syrup or may alternatively be recycled to the slurry tank as “backset”. Evaporator condensate may either be forwarded to a methanator before being discharged and/or may be recycled to the slurry tank as “cook water”. The syrup may be blended into DDG or added to the wet cake before or during the drying process, which can comprise one or more dryers in sequence, to produce DDGS (Distillers Dried Grain with Solubles). Syrup typically contains about 25 to 35% solids. Oil can also be extracted from the thin stillage and/or syrup as a by-product for use in biodiesel production, as a feed or food additive or product, or other biorenewable products.

Yeasts which are used for production of ethanol for use as fuel, such as in the corn ethanol industry, require several characteristics to ensure cost effective production of the ethanol. These characteristics include ethanol tolerance, low by-product yield, rapid fermentation, and the ability to limit the amount of residual sugars remaining in the ferment. Such characteristics have a marked effect on the viability of the industrial process.

Despite significant improvement of ethanol production processes over the past decade there is still a desire and need for providing improved processes of ethanol fermentation from starch containing material in an economically and commercially relevant scale.

### SUMMARY OF THE INVENTION

The performance of ethanol fermentation of fermentable sugars produced from liquefied starch-containing material may be negatively impacted if the yeast is challenged by lactic acid, or other inhibitory compounds produced from infectious organisms. For the yeast to be the most productive in fermentation, it is imperative to shorten yeast lag phase and begin ethanol production at a faster rate.

The present invention provides a solution to these problems by using a peroxidase to accelerate yeast growth and/or productivity, for instance, to increase ethanol titers early in the fermentation process, resulting in an overall reduction in lactic acid titers during fermentation, especially when a fermentation is challenged by an infection. The processes and compositions of the invention can also be used to culture, cultivate, propagate, or produce yeast by enhancing yeast growth and/or productivity.

In an aspect, the present invention relates to a process for enhancing yeast growth and/or productivity, the process comprising contacting yeast with an effective amount of a peroxidase or peroxidase composition.

In an aspect, the present invention relates to a process for the production of yeast, comprising cultivating yeast in the presence of an effective amount of a peroxidase or peroxidase composition under conditions conducive for yeast growth.

In some embodiments, the growth of the yeast is increased by 10% to 50% in comparison to growth of yeast not contacted with the polypeptide. In some embodiments, the productivity of the yeast is increased by 10% to 50% in comparison to productivity of yeast not contacted with the polypeptide.

In an aspect, the present invention relates to a composition comprising yeast produced according to a presently disclosed process and a component selected from a surfactant, an emulsifier, a gum, a swelling agent, an antioxidant, a processing aid, and/or any combination thereof. In some embodiments, the composition is formulated as a cream yeast, a crumbled yeast, a compressed yeast, or an active dry yeast. In an embodiment, the composition is formulated as an inactive dry yeast (e.g., nutritional yeast).

In an aspect, the present invention relates to a container comprising a presently disclosed yeast composition. In some embodiments, the container is selected from a tote, a dosage skid, a package, a sack, or a fermentation vessel.

In an aspect, the present invention relates to a process for propagating yeast for bioproduct production in a biofuel fermentation system, the process comprising introducing a peroxidase or peroxidase composition to a biofuel fermentation system, wherein the fermentation system comprises one or more fermentation vessels, pipes and/or components. In an embodiment, the peroxidase or peroxidase composition is added at a concentration sufficient to enhance yeast growth and/or productivity in the biofuel fermentation system.

In an embodiment, at least one of the fermentation vessels is a fermentation tank and the peroxidase or peroxidase composition is introduced into the fermentation tank. In some embodiments, the peroxidase or peroxidase composition is introduced into the fermentation tank within the first 6 hours of fermentation. In some embodiments, the tate at which ethanol is produced within the first 24 hours of fermentation is increased by from 10% to 50% compared to the rate at which ethanol is produced within the first 24 hours without the peroxidase or peroxidase composition. In some embodiment, the growth of yeast within the first 24 hours of fermentation is increased by from 10% to 50% compared to the growth of yeast within the first 24 hours of fermentation without the peroxidase or peroxidase composition.

In an embodiment, at least one of the fermentation vessels is a yeast propagation tank and the peroxidase or peroxidase composition is introduced into the yeast propagation tank. In some embodiments, the rate at which ethanol is produced within the first 24 hours of fermentation is increased by from 10% to 50% compared to the amount of ethanol produced within the first 24 hours without the peroxidase. In some embodiments, the growth of yeast after 24 hours of propagation is increased by from 10% to 50% in the presence of the peroxidase compared to the growth of yeast over the same period of propagation without the peroxidase.

In an embodiment, the process includes a step of adding yeast to the propagation tank or to the fermentation vessel. In some embodiments, the yeast is contacted with a peroxidase prior to being added to the propagation tank or the fermentation vessel.

In an embodiment, the biofuel is ethanol.

In an aspect, the invention relates to a process for producing a fermentation product from a starch-containing material, the process comprising: a) liquefying a starch-containing material in the presence of an alpha-amylase to form a liquefied mash; b) saccharifying the liquefied mash using a carbohydrate source generating enzyme to produce a fermentable sugar; c) fermenting the sugar using a fermenting organism under conditions suitable to produce the fermentation product, wherein a peroxidase is added before or during saccharifying step b) and/or fermenting step c).

In some embodiments, steps b) and c) are carried out simultaneously. In some embodiments, a slurry of the starch containing material is heated to above the gelatinization temperature. In some embodiments, a peroxidase is added during liquefaction. In some embodiments, a peroxidase is added during saccharification, wherein the peroxidase is optionally added within the first two hours of saccharification. In some embodiments, a peroxidase is added during fermentation, wherein the peroxidase is optionally added within the first six hours of fermentation. In an embodiment, the peroxidase is introduced just after liquefaction and before the fermentation tank or propagation tank. In an embodiment, the peroxidase is introduced at any point of the mash cooling system. In an embodiment, the peroxidase is added to a heat exchanger. In an embodiment, the peroxidase is added to a mixing tank. In some embodiments, the fermentation product is an alcohol, preferably ethanol.

In some embodiments, the fermenting organism is yeast.

In some embodiments, the yeast belongs to a genus selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera. In some embodiments, the yeast is Saccharomyces cerevisiae, Saccharomyces pastorianus (carlsbergiensis), Kluyveromyces lactis, Kluyveromyces fragilis, Fusarium oxysporum, or any combination thereof. In some embodiments, the yeast is Saccharomyces cerevisiae. In some embodiments, the yeast comprises a heterologous polynucleotide encoding an enzyme selected from an alpha-amylase, a glucoamylase, or a protease.

In some embodiments, the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase. In some embodiments, the peroxidase is derived from a microorganism, such as a fungal organism, such a yeast or filamentous fungi, or bacteria; or plant. In some embodiments, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

In an aspect, the invention relates to use of a peroxidase for propagating yeast. In an aspect, the invention relates to use of a peroxidase for increasing the growth and/or productivity of yeast.

In an aspect, the invention relates to use of a peroxidase for increasing the rate at which ethanol is produced within the first 24 hours of fermentation during a biofuel (e.g., ethanol) production process.

In an aspect, the invention relates to use of a peroxidase for reducing lactic acid titers during the fermentation or simultaneous saccharification and fermentation steps of a biofuel (e.g., ethanol) production process.

In an aspect, the present invention relates to the use of a peroxidase for reducing the levels of lactic acid during fermentation in an ethanol production process.

In an aspect, the present invention relates to the use of a peroxidase for reducing the levels of lactic acid during yeast propagation.

### BRIEF DESCRIPTION OF THE DRAWING

FIG. 1 shows an exemplary dry-grind ethanol production process.

FIG. 2 shows ethanol titers (g/L) after 24 hours of fermentation of a liquefied corn mash having 20% dried solids (DS) content in the presence of various peroxidases compared to a control lacking peroxidase and a control in which only penicillin was used.

FIG. 3 shows lactic acid titers (g/L) after 24 hours of fermentation of a liquefied corn mash having 20% dried solids content in the presence of various peroxidases compared to a control lacking peroxidase and a control in which only penicillin was used.

FIG. 4 shows ethanol titers (g/L) after 24 hours of fermentation of a liquefied corn mash having 20% dried solids (DS) content in the presence of various peroxidases compared to a control lacking peroxidase and a control in which only penicillin was used.

FIG. 5 shows lactic acid titers (g/L) after 24 hours of fermentation of a liquefied corn mash having 20% dried solids content in the presence of various peroxidases compared to a control lacking peroxidase and a control in which only penicillin was used.

FIG. 6 shows ethanol titers (g/L) after 60 hours of fermentation of a liquefied corn mash having 32% dried solids (DS) content in the presence of various peroxidases compared to a control lacking peroxidase and a control in which only penicillin was used.

FIG. 7 shows lactic acid titers (g/L) after 60 hours of fermentation of a liquefied corn mash having 32% dried solids content in the presence of various peroxidases compared to a control lacking peroxidase and a control in which only penicillin was used.

FIG. 8A, FIG. 8B, FIG. 8C, FIG. 8D and FIG. 8E are citation images showing yeast cell growth in a sterile nutrient medium without peroxidase (control; FIG. 8A) and in the presence of increasing concentrations of peroxidase (5 uL T.a. Catalase (FIG. 8B); 25 uL T.a. Catalase (FIG. 8C); 50 uL T.a. Catalase (FIG. 8D); and 200 uL T.a. Catalase (FIG. 8E)).

FIG. 9 is a graph showing the average cell counts of the yeast shown in FIG. 8A, FIG. 8B, FIG. 8C, FIG. 8D and FIG. 8E, as counted using Cytation software.

FIG. 10 is a graph showing the effects of certain peroxidases on yeast growth in a 14 L propagation compared to a baseline control without peroxidase.

FIG. 11A is a graph showing glucose titers (g/L) after 6 hours of propagation in 20% DS with and without peroxidase treatment.

FIG. 11B is a graph showing ethanol titers (g/L) after 6 hours of propagation in 20% DS with and without peroxidase treatment.

FIG. 12 is a graph showing early fermentation kinetics of yeast treated with increasing concentrations (10 uL, 50 uL, 100 uL and 450 uL) of a peroxidase compared to controls, as measured by an Ankom pressure monitor.

FIG. 13A is a graph showing lactic acid titers (g/L) after 60 hours of fermentation at 32% DS, following propagation of yeast in the presence of various concentrations of peroxidase.

FIG. 13B is a graph showing ethanol titers (g/L) after 60 hours of fermentation at 32% DS, following propagation of yeast in the presence of various concentrations of peroxidase.

FIG. 13C is a graph showing DP2 titers (g/L) after 60 hours fermentation at 32% DS, following propagation of yeast in the presence of various concentrations of peroxidase.

### DEFINITIONS

Unless defined otherwise or clearly indicated by context, all technical and scientific terms used herein have the same meaning as commonly understood by one of ordinary skill in the art.

Alpha-Amylases: Alpha-amylases (E.C. 3.2.1.1) are a group of enzymes which catalyze the hydrolysis of starch and other linear and branched 1,4 glucosidic oligo- and polysaccharides. The skilled person will know how to determine alpha-amylase activity. It may be determined according to the procedure described in the Examples, e.g., by the PNP-G7 assay or the EnzCheck assay.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Catalase: The term “catalase” means a hydrogen-peroxide:hydrogen-peroxide oxidoreductase (EC 1.11.1.6) that catalyzes the conversion of 2H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

cDNA: The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enhancing activity: The term “cellulolytic enhancing activity” is defined herein as a biological activity that enhances the hydrolysis of a cellulosic material by polypeptides having cellulolytic activity. For purposes of the present invention, cellulolytic enhancing activity is determined by measuring the increase in reducing sugars or the increase of the total of cellobiose and glucose from the hydrolysis of a cellulosic material by cellulolytic protein under the following conditions: 1-50 mg of total protein/g of cellulose in PCS, wherein total protein is comprised of 50-99.5% w/w cellulolytic protein and 0.5-50% w/w protein of cellulolytic enhancing activity for 1-7 day at 50-65° C. compared to a control hydrolysis with equal total protein loading without cellulolytic enhancing activity (1-50 mg of cellulolytic protein/g of cellulose in PCS). In a preferred aspect, a mixture of CELLUCLAST® 1.5 L (Novozymes A/S, Bagsværd, Denmark) in the presence of 3% of total protein weight Aspergillus oryzae beta-glucosidase (recombinantly produced in Aspergillus oryzae according to WO 02/095014) or 3% of total protein weight Aspergillus fumigatus beta-glucosidase (recombinantly produced in Aspergillus oryzae as described in WO 02/095014) of cellulase protein loading is used as the source of the cellulolytic activity.

The polypeptides having cellulolytic enhancing activity enhance the hydrolysis of a cellulosic material catalyzed by proteins having cellulolytic activity by reducing the amount of cellulolytic enzyme required to reach the same degree of hydrolysis preferably at least 1.01-fold, more preferably at least 1.05-fold, more preferably at least 1.10-fold, more preferably at least 1.25-fold, more preferably at least 1.5-fold, more preferably at least 2-fold, more preferably at least 3-fold, more preferably at least 4-fold, more preferably at least 5-fold, even more preferably at least 10-fold, and most preferably at least 20-fold.

Cellulolytic enzyme, cellulolytic composition, or cellulase: The term “cellulolytic enzyme”, “cellulolytic composition”, or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

Cellulolytic enzyme activity is determined by measuring the increase in hydrolysis of a cellulosic material by cellulolytic enzyme(s) under the following conditions: 1-50 mg of cellulolytic enzyme protein/g of cellulose in Pretreated Corn Stover (“PCS”) (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, Calif., USA).

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences that provide for its expression.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. In some instances, the fermentation medium is derived from a natural source, such as sugar cane, starch, or cellulose. The term fermentation medium is understood herein to refer to a medium before the fermenting organism is added, such as, a medium resulting from a saccharification process, as well as a medium used in a simultaneous saccharification and fermentation process (SSF).

Fermenting organism: The term “fermenting organism” refers to any organism, including bacterial and fungal organisms, such as yeast and filamentous fungi, suitable for producing a desired fermentation product. Suitable fermenting organisms are able to ferment, i.e., convert, fermentable sugars, such as arabinose, fructose, glucose, maltose, mannose, or xylose, directly or indirectly into the desired fermentation product.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

Glucoamylase: The term “glucoamylase” (1,4-alpha-D-glucan glucohydrolase, EC 3.2.1.3) is defined as an enzyme, which catalyzes the release of D-glucose from the non-reducing ends of starch or related oligo- and polysaccharide molecules. For purposes of the present invention, glucoamylase activity is determined according to the procedure described in the Examples herein. The Glucoamylase Unit (AGU) is defined as the amount of enzyme, which hydrolyses 1 micromole maltose per minute under the standard conditions 37° C., pH 4.3, substrate: maltose 23.2 mM, buffer: acetate 0.1 M, reaction time 5 minutes.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Homologous sequence: The term “homologous sequence” is defined herein as a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with a polypeptide of interest.

Host cell: The term “host cell” means any cell type that is susceptible to transformation, transfection, transduction, and the like with a nucleic acid construct or expression vector comprising a polynucleotide described herein (e.g., a polynucleotide encoding an alpha-amylase, glucoamylase, or protease). The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment).

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” is defined herein as a polypeptide having biological activity that is in its final form following translation and any post-translational modifications, such as N-terminal processing, C-terminal truncation, glycosylation, phosphorylation, etc. In an embodiment, the mature polypeptide is amino acids 20 to 717 of the polypeptide of SEQ ID NO: 1. Amino acids 1 to 19 of the polypeptide of SEQ ID NO: 1 is a predicted signal peptide. In an embodiment, the mature polypeptide is amino acids 23 to 351 of the polypeptide of SEQ ID NO: 3. Amino acids 1 to 22 of the polypeptide of SEQ ID NO: 3 is predicted signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” is defined herein as a nucleotide sequence that encodes a mature polypeptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Modification: The term “modification” means herein any chemical modification of a polypeptide, as well as genetic manipulation of the DNA encoding the polypeptide. The modification can be a substitution, a deletion and/or an insertion of one or more (several) amino acids as well as replacements of one or more (several) amino acid side chains.

Mutant: The term “mutant” means a polynucleotide encoding a variant.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence.

Operably linked: The term “operably linked” means a configuration in which a control sequence is placed at an appropriate position relative to the coding sequence of a polynucleotide such that the control sequence directs expression of the coding sequence.

Peroxidase: The term “Peroxidase” is defined herein includes enzymes having peroxidase activity and Peroxide-decomposing enzymes.

Peroxidase activity: The term “peroxidase activity” is defined herein as an enzyme activity that converts a peroxide, e.g., hydrogen peroxide, to a less oxidative species, e.g., water. It is understood herein that a polypeptide having peroxidase activity encompasses a peroxide-decomposing enzyme (defined below) and is used interchangeably herein with “peroxidase”.

Peroxide-decomposing enzyme: The term “peroxide-decomposing enzyme” is defined herein as an donor:peroxide oxidoreductase (E.C. number 1.11.1.x) that catalyzes the reaction reduced substrate(2e⁻)+ROOR′→oxidized substrate+ROH+R′OH; such as horseradish peroxidase that catalyzes the reaction phenol+H₂O₂→quinone+H₂O, and catalase that catalyzes the reaction H₂O₂+H₂O₂→O₂+2H₂O. In addition to hydrogen peroxide, other peroxides may also be decomposed by these enzymes.

Polypeptide fragment: The term “polypeptide fragment” is defined herein as a polypeptide having one or more (several) amino acids deleted from the amino and/or carboxyl terminus of a mature polypeptide or a homologous sequence thereof, wherein the fragment has biological activity.

Pretreated corn stover: The term “Pretreated Corn Stover” or “PCS” means a cellulosic-containing material derived from corn stover by treatment with heat and dilute sulfuric acid, alkaline pretreatment, neutral pretreatment, or any pretreatment known in the art.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 223: 1-5 (1994); Eur. J. Biochem. 232: 1-6 (1995); Eur. J. Biochem. 237: 1-5 (1996); Eur. J. Biochem. 250: 1-6 (1997); and Eur. J. Biochem. 264: 610-650 (1999); respectively. The term “subtilases” refer to a sub-group of serine protease according to Siezen et al., 1991, Protein Engng. 4: 719-737 and Siezen et al., 1997, Protein Science 6: 501-523.

Proteases are classified on the basis of their catalytic mechanism into the following groups: Serine proteases (S), Cysteine proteases (C), Aspartic proteases (A), Metalloproteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

Polypeptides having protease activity, or proteases, are sometimes also designated peptidases, proteinases, peptide hydrolases, or proteolytic enzymes. Proteases may be of the exo-type (exopeptidases) that hydrolyse peptides starting at either end thereof, or of the endo-type that act internally in polypeptide chains (endopeptidases).

In particular embodiments, the proteases for use in the processes of the invention are selected from the group consisting of:
(a) proteases belonging to the EC 3.4.24 metalloendopeptidases;
(b) metalloproteases belonging to the M group of the above Handbook;
(c) metalloproteases not yet assigned to clans (designation: Clan MX), or belonging to either one of clans MA, MB, MC, MD, ME, MF, MG, MH (as defined at pp. 989-991 of the above Handbook);
(d) other families of metalloproteases (as defined at pp. 1448-1452 of the above Handbook);
(e) metalloproteases with a HEXXH motif;
(f) metalloproteases with an HEFTH motif;
(g) metalloproteases belonging to either one of families M3, M26, M27, M32, M34, M35, M36, M41, M43, or M47 (as defined at pp. 1448-1452 of the above Handbook); and
(h) metalloproteases belonging to family M35 (as defined at pp. 1492-1495 of the above Handbook).

Protease activity: The term “protease activity” means proteolytic activity (EC 3.4). There are several protease activity types such as trypsin-like proteases cleaving at the carboxyterminal side of Arg and Lys residues and chymotrypsin-like proteases cleaving at the carboxyterminal side of hydrophobic amino acid residues.

Protease activity can be measured using any assay, in which a substrate is employed, that includes peptide bonds relevant for the specificity of the protease in question. Assay-pH and assay-temperature are likewise to be adapted to the protease in question. Examples of assay-pH-values are pH 2, 3, 4, 5, 6, 7, 8, 9, 10, 11, or 12. Examples of assay-temperatures are 15, 20, 25, 30, 35, 37, 40, 45, 50, 55, 60, 65, 70, 80, 90, or 95° C. Examples of general protease substrates are casein, bovine serum albumin and haemoglobin. In the classical Anson and Mirsky method, denatured haemoglobin is used as substrate and after the assay incubation with the protease in question, the amount of trichloroacetic acid soluble haemoglobin is determined as a measurement of protease activity (Anson, M. L. and Mirsky, A. E., 1932, J. Gen. Physiol. 16: 59 and Anson, M. L., 1938, J. Gen. Physiol. 22: 79).

For the purpose of the present invention, protease activity may be determined using assays which are described in “Materials and Methods”, such as the Kinetic Suc-AAPF-pNA assay, Protazyme AK assay, Kinetic Suc-AAPX-pNA assay and o-Phthaldialdehyde (OPA). For the Protazyme AK assay, insoluble Protazyme AK (Azurine-Crosslinked Casein) substrate liberates a blue colour when incubated with the protease and the colour is determined as a measurement of protease activity. For the Suc-AAPF-pNA assay, the colourless Suc-AAPF-pNA substrate liberates yellow paranitroaniline when incubated with the protease and the yellow colour is determined as a measurement of protease activity.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Referenced Sequence−Total Number of Gaps in Alignment)

Signal peptide: The term “signal peptide” is defined herein as a peptide linked (fused) in frame to the amino terminus of a polypeptide having biological activity and directs the polypeptide into the cell's secretory pathway.

Subsequence: The term “subsequence” is defined herein as a nucleotide sequence having one or more (several) nucleotides deleted from the 5′ and/or 3′ end of a mature polypeptide coding sequence or a homologous sequence thereof, wherein the subsequence encodes a polypeptide fragment having biological activity.

Trehalase: The term “trehalase” means an enzyme which degrades trehalose into its unit monosaccharides (i.e., glucose). Trehalases are classified in EC 3.2.1.28 (alpha,alpha-trehalase) and EC. 3.2.1.93 (alpha,alpha-phosphotrehalase). The EC classes are based on recommendations of the Nomenclature Committee of the International Union of Biochemistry and Molecular Biology (IUBMB). Description of EC classes can be found on the internet, e.g., on “http://www.expasy.orq/enzyme/”. Trehalases are enzymes that catalyze the following reactions:

### EC 3.2.1.28:

Alpha,alpha-trehalose+H₂O⇔2 D-glucose;

### EC 3.2.1. 93:

Alpha,alpha-trehalose 6-phosphate+H₂O⇔D-glucose+D-glucose 6-phosphate.

For purposes of the present invention, trehalase activity may be determined according to the trehalase assay procedure described below.

### Principle:

Trehalose+H₂O^{Trehalase}>2 Glucose
T=37° C., pH=5.7, A340 nm, Light path=1 cm

### Spectrophotometric Stop Rate Determination

### Unit Definition:

One unit will convert 1.0 mmole of trehalose to 2.0 mmoles of glucose per minute at pH 5.7 at 37° C. (liberated glucose determined at pH 7.5).

### (See Dahlqvist, A. (1968) Analytical Biochemistry 22, 99-107)

Variant: The term “variant” means a polypeptide having enzyme or enzyme enhancing activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. Variants of the invention can have at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a reference polypeptide (e.g., an enzyme described herein). In some embodiments, the variant has less than 100% sequence identity toe the amino acid sequence of a reference polypeptide (e.g., an enzyme described herein).

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value, and can include a range of plus or minus two standard deviations around the stated value.

Likewise, reference to a gene or polypeptide that is “derived from” another gene or polypeptide X, includes the gene or polypeptide X.

As used herein and in the appended claims, the singular forms “a,” “or,” and “the” include plural referents unless the context clearly dictates otherwise.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

### DESCRIPTION OF THE INVENTION

The present invention relates to use of peroxidases for enhancing yeast growth and/or productivity, for example during yeast propagation, such as especially while propagating yeast for bioproduct production in a biofuel fermentation system. The present invention also relates to processes for producing a fermentation product from a starch-containing material using a fermenting organism, wherein a peroxidase is added during yeast propagation and/or during fermentation.

The inventors have surprisingly found that yeast growth is increased when yeast are cultivated in the presence of peroxidase. The data presented herein unexpectedly demonstrates that peroxidases improve early yeast kinetics early during propagation and/or fermentation, and in particular that yeast propagated with peroxidase consume more glucose and significantly increase ethanol titers within the first six hours of propagation compared to control propagations lacking peroxidase. Surprisingly, when such propagated yeast were transferred into fermentation and yeast were challenged with infection, the peroxidase treated yeast were able to outcompete the infection more productively as measured by reduced lactic acid titers.

I. Enhancing Fermenting Organism Growth and/or Productivity

In an aspect, the invention relates to a process for enhancing fermenting organism growth and/or productivity, the process comprising contacting a fermenting organism with an effective amount of a peroxidase or a composition comprising a polypeptide having peroxidase activity.

In an embodiment, the invention relates to a process for enhancing yeast growth and/or productivity, the process comprising contacting yeast with an effective amount of a peroxidase or a composition comprising a polypeptide having peroxidase activity.

As used herein, the phrases “enhancing fermenting organism growth and/or productivity” and “enhancing yeast growth and/or productivity“ ” encompass enhancing fermenting organism growth/yeast growth, enhancing fermenting organism productivity/yeast productivity, or enhancing both fermenting organism growth/yeast growth and enhancing fermenting organism productivity/yeast productivity.

The phrase “enhancing yeast growth” encompasses increasing the growth rate and biomass yield (e.g., increase in the number of yeast cells in a population) during both aerobic and anerobic fermentation. It should be appreciated that “increasing the growth rate” encompasses an increase in the sustained growth rate and/or an increase in the maximum instantaneous growth rate. It is to be understood that the definition of and following description of “enhancing yeast growth” is equally applicable to the phrase “enhancing fermenting organism growth” except the following description is focused on yeast for the sake of brevity. It is to be further understood in the context of the disclosed processes that the increase in the growth rate and/or biomass yield of yeast contacted with a peroxidase or peroxidase composition is assessed relative to yeast under the same or similar conditions but not contacted with a peroxidase or peroxidase composition of the invention.

The peroxidases, compositions, and processes comprising the peroxidase can result in a detectable increase in yeast biomass yield. In various aspects of the invention, the biomass yield of yeast contacted with the peroxidase or peroxidase composition is increased by at least 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, or 5-fold in comparison to growth of yeast under the same or similar conditions but not contacted with the peroxidase or peroxidase composition.

The peroxidases, compositions, and processes comprising the peroxidase can result in a detectable increase in the rate of yeast growth. In various aspects of the invention, the growth rate of yeast contacted with the peroxidase or peroxidase composition is increased by at least 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, or 5-fold in comparison to the growth rate of yeast under the same or similar conditions but not contacted with the peroxidase or peroxidase composition.

The term “enhancing yeast productivity” encompasses an increase in the rate at which a fermentation product is produced by yeast, an increase in the absolute titers of the fermentation product produced by yeast, as well as an increase in the rate or amount of nutrient consumed by the yeast. For example, the peroxidases, compositions and processes comprising peroxidase can increase the rate in yeast metabolite production and/or yeast enzyme production (e.g., heterologous enzyme expression). It is to be understood that the definition of and following description of “enhancing yeast productivity” is equally applicable to the phrase “enhancing fermenting organism productivity” except the following description is focused on yeast for the sake of brevity. It is to be further understood in the context of the disclosed processes that the increases in the rate and absolute titers of the yeast fermentation product, as well as increase in the rate or amount of nutrient consumed by the yeast, are assessed relative to the rate and absolute titers of the yeast fermentation product and rate or amount of nutrient consumed by yeast under the same or similar conditions but not contacted with a peroxidase of the invention. The peroxidases and compositions and processes involving the peroxidases result in a statistically significant increase in yeast productivity.

In aspects of the invention, the productivity of the yeast contacted with the peroxidase or peroxidase composition is increased by 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, or 5-fold in comparison to productivity of yeast under the same or similar conditions but not contacted with the peroxidase or peroxidase composition.

In aspects of the invention, the rate at which a fermentation product is produced by yeast contacted with a peroxidase or peroxidase composition of the invention is increased by 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold, or 10-fold in comparison to the rate at which the fermentation product is produced by yeast under the same or similar conditions but not contacted with the peroxidase or peroxidase composition.

In aspects of the invention, the absolute titer of the fermentation product produced by yeast contacted with a peroxidase or peroxidase composition of the invention is increased by 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold, or 10-fold in comparison to the titer of the fermentation product produced by yeast under the same or similar conditions but not contacted with the peroxidase or peroxidase composition.

In an embodiment, the rate at which ethanol is produced by yeast contacted with a peroxidase or peroxidase composition of the invention is increased by 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold, or 10-fold in comparison to the rate at which the ethanol is produced by yeast under the same or similar conditions but not contacted with the peroxidase or peroxidase composition.

In an embodiment, the rate at which glucose is consumed, or the amount of glucose consumed, by yeast contacted with a peroxidase or peroxidase composition of the invention is increased by 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 1-fold, 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold, or 10-fold in comparison to the rate at which glucose is consumed, or the amount of glucose consumed, by yeast under the same or similar conditions but not contacted with a peroxidase or peroxidase composition.

The term “contacting” encompasses any method in which a peroxidase or composition comprising a peroxidase is placed into physical contact with yeast or an environment in which the yeast reside. For example the peroxidase or peroxidase composition can be formulated with a yeast composition (e.g., a cream yeast formulation), the peroxidase or peroxidase composition can be added to a medium comprising yeast (e.g., a nutrient medium), the peroxidase or peroxidase composition can be added to a fermentation vessel comprising yeast (e.g., a yeast propagation tank, a bioreactor, etc.), or the peroxidase or peroxidase composition can be added to a container comprising yeast (e.g., a tote, a dosage skid, etc.).

The term “effective amount” means an amount which will enhance the growth and/or productivity of yeast contacted with the peroxidase or peroxidase composition by at least a statistically significant amount compared to growth and/or productivity of yeast under the same conditions but not contacted with the peroxidase or peroxidase composition. The effective amount will depend on various factors known to those of ordinary skill in the art. Such factors include, but are not limited to, the scale of the fermentation or propagation, the number of propagation cycles, the starting yeast density, desired final yeast density, contents of the growth or fermentation medium, volume of the bioreactor or fermentation vessel, the type of fermentation (e.g., batch mode, fed-batch mode, etc.), reaction time, reaction temperature, and reaction pH. Effective amounts of peroxidase range from 0.01 μg to 5000 μg concentrated product, preferably from 0.10 μg to 2500 μg concentrated product, more preferably from 1 μg to 1000 μg concentrated product, and even more preferably from 10 μg to 500 μg concentrated product. In an embodiment, an effective amount of peroxidase ranges from 10 μg to 450 μg concentrated product.

Any fermenting organism, such as especially the fermenting organisms described herein under the heading “Fermenting organism” can be used in the processes of enhancing the growth and/or productivity of a fermenting organism. In an embodiment, the fermenting organism is yeast. In an embodiment, the yeast belongs to a genus selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera. In an embodiment, the yeast is Saccharomyces cerevisiae, Saccharomyces pastorianus (carlsbergiensis), Kluyveromyces lactis, Kluyveromyces fragilis, Fusarium oxysporum, or any combination thereof. In an embodiment, the yeast is Saccharomyces cerevisiae. In an embodiment, the yeast comprises a heterologous polynucleotide encoding an enzyme selected from an alpha-amylase, a glucoamylase, or a protease.

In an embodiment, the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase. In an embodiment, the peroxidase is derived from a microorganism, such as a fungal organism, such a yeast or filamentous fungi, or bacteria; or plant. In an embodiment, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

### II. Production of Fermenting Organisms

Aspects of the invention relate to processes for the production of fermenting organisms comprising cultivating fermenting organisms in the presence of a peroxidase or composition comprising a polypeptide having peroxidase activity under conditions conducive for growth of the fermenting organism.

In an aspect, the invention relates to a process for the production of yeast comprising cultivating yeast in the presence of a peroxidase or composition comprising a polypeptide having peroxidase activity under conditions conducive for yeast growth. The process contemplates production of yeast on any scale (e.g., commercial scale). Those skilled in the art will appreciate that there are a variety of conditions conducive for yeast growth that can be optimized to ensure optimal growth of a particular yeast strain (e.g., for commercial production). For example, a pure yeast culture can be cultivated in several stages of various scale up before reaching the main production stage. Throughout each successive stage bioreactor sizes can be used depending on the desired amount of yeast to be produced. The examples below describe exemplary conditions for small scale yeast production. In an embodiment, main production is carried out as a fed-batch propagation under aerobic conditions in an aqueous growth medium containing an assimilable source of nitrogen, vitamins, trace metals, salts and a continuous addition of a carbohydrate source. Preferably, pH of the broth is controlled from 4.0 to 6.0 with aqueous ammonia and/or dilute base. Temperature can be maintained between 25° C. and 38° C. throughout propagation. Carbohydrate feed rates are selected to achieve a high specific growth rate such that feed rates to not exceed the oxygen transfer or cooling capacity of the propagator. Propagation may take between 30 to 50 hours and completes with a broth containing between 60 to 120% dry yeast solids. Following propagation, yeast cells are concentrated for further processing into the desired product (e.g., cream yeast, crumbled yeast, active or inactive dry yeast, compressed yeast, etc.) depending on the application (e.g., baking, brewing, biofuel fermentation, etc.).

### III. Fermenting Organism Compositions

Aspects of the invention relate to compositions comprising a fermenting organism (e.g., a fermenting organism described herein) and a naturally occurring and/or non-naturally occurring component. In an embodiment, the invention relates to a composition comprising a yeast strain (e.g., a yeast strain produced according to a process described herein) and a component selected from a surfactant, an emulsifier, a gum, a swelling agent, an antioxidant, a processing aid, and/or any combination thereof. In various aspects and embodiments, the fermenting organism in the composition is a fermenting organism produced by contacting, cultivating, culturing, producing and/or propagating the fermenting organism with a peroxidase or a peroxidase composition. In various aspects and embodiments, the fermenting organism in the composition is a yeast strain produced by contacting, cultivating, culturing, producing and/or propagating the yeast with a peroxidase or peroxidase composition. In various aspects and embodiments, the composition comprising the fermenting organism (e.g., yeast strain described herein) and the component selected from a surfactant, emulsifier, gum, swelling aent, antioxidant, processing aid, and/or any combination thereof further comprises a peroxidase.

The fermenting organism of the composition may be in any viable form, including crumbled, dry, including active dry and instant, compressed, cream (liquid) form etc. In one embodiment, the fermenting organism (e.g., a Saccharomyces cerevisiae yeast strain) is dry yeast, such as active dry yeast or instant yeast. In one embodiment, the fermenting organism (e.g., a Saccharomyces cerevisiae yeast strain) is crumbled yeast. In one embodiment, the fermenting organism (e.g., a Saccharomyces cerevisiae yeast strain) is compressed yeast. In one embodiment, the fermenting organism (e.g., a Saccharomyces cerevisiae yeast strain) is cream yeast.

In one embodiment is a composition comprising a fermenting organism described herein (e.g., a Saccharomyces cerevisiae yeast strain), and one or more of the component selected from a surfactant, an emulsifier, a gum, a swelling agent, an antioxidant, a processing aid, and/or any combination thereof.

The compositions described herein may comprise a fermenting organism described herein (e.g., a Saccharomyces cerevisiae yeast strain) and any suitable surfactants. In some embodiments, the composition comprising the fermenting organism and the surfactant further includes a peroxidase. In one embodiment, the surfactant(s) is/are an anionic surfactant, cationic surfactant, and/or nonionic surfactant.

The compositions described herein may comprise a fermenting organism described herein (e.g., a Saccharomyces cerevisiae yeast strain) and any suitable emulsifier. In some embodiments, the composition comprising the fermenting organism and the emulsifier further includes a peroxidase. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a fermenting organism described herein (e.g., a Saccharomyces cerevisiae yeast strain) and any suitable gum. In some embodiments, the composition comprising the fermenting organism and the gum further includes a peroxidase. In one embodiment, the gum is selected from the group of carob, guar, tragacanth, arabic, xanthan and acacia gum, in particular for cream, compressed and dry yeast.

The compositions described herein may comprise a fermenting organism described herein (e.g., a Saccharomyces cerevisiae yeast strain) and any suitable swelling agent. In some embodiments, the composition comprising the fermenting organism and the swelling agent further includes a peroxidase. In one embodiment, the swelling agent is methyl cellulose or carboxymethyl cellulose.

The compositions described herein may comprise a fermenting organism described herein (e.g., a Saccharomyces cerevisiae yeast strain) and any suitable anti-oxidant. In some embodiments, the composition comprising the fermenting organism and the anti-oxidant further includes a peroxidase. In one embodiment, the antioxidant is butylated hydroxyanisol (BHA) and/or butylated hydroxytoluene (BHT), or ascorbic acid (vitamin C), particular for active dry yeast.

### IV. Containers

Aspects of the invention relate to a container comprising a fermenting organism composition described herein, such as especially a yeast composition described in Section III herein.

The present invention contemplates the use of any container into which a fermenting organism (e.g., a fermenting organism described herein, e.g., a yeast composition comprising yeast contacted, cultivated, cultured, produced and/or propagated in the presence of a peroxidase). Examples of suitable containers include, without limitation, a tote, a dosage skid, a package, a sack, and a fermentation vessel, such as a propagation or fermentation tank. In an embodiment, the container is a tote. In an embodiment, the container is a dosage skid. In an embodiment, the container is a package. In an embodiment, the container is a sack. In an embodiment, the container is a propagation tank.

In an embodiment, the container is a fermentation tank.

### V. Propagating Yeast for Bioproduct Production in a Biofuel Fermentation System

In an aspect the invention relates to a process for propagating yeast for bioproduct production in a biofuel fermentation system, the process comprising introducing a peroxidase or peroxidase composition to a biofuel fermentation system. The terms “bioproduct” and “fermentation product” are used interchangeably herein. The peroxidase can be added at a concentration sufficient to enhance yeast growth and/or productivity in the biofuel fermentation system (i.e., an effective amount).

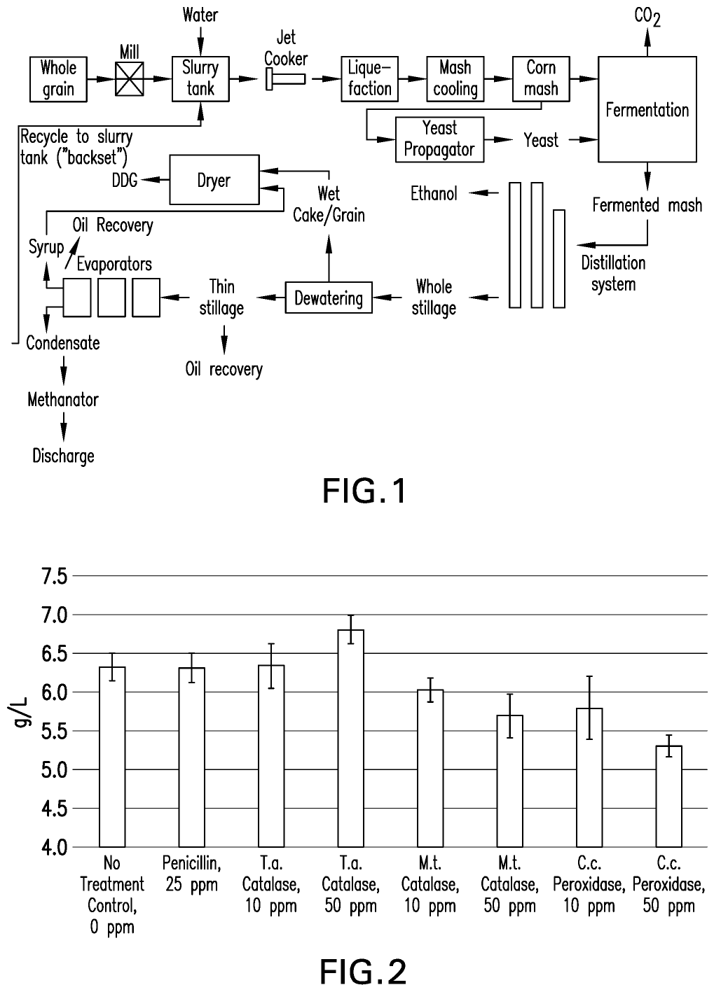

Systems and methods for biofuel fermentation are well known in the art. The fermentation system may include one or more fermentation vessels, pipes, and/or components, which are configured to perform a fermentation product production process, such as the exemplary dry-grind ethanol production process shown in FIG. 1.

Those skilled in the art will appreciate that the peroxidase or peroxidase may be introduced into, or prior to, the propagation or fermentation system at a variety of different locations.

In an embodiment, at least one of the fermentation vessels in the fermentation system is a fermentation tank and the peroxidase or peroxidase composition is introduced into the fermentation tank. In an embodiment, the peroxidase or peroxidase composition is introduced to the fermentation tank before saccharification begins. In an embodiment, the peroxidase or peroxidase composition is introduced to the fermentation tank before fermentation begins. In an embodiment, the peroxidase or peroxidase composition is introduced to the fermentation tank before simultaneous saccharification and fermentation begins. In an embodiment, the peroxidase or peroxidase composition is added within the first minute, first five minutes, first 10 minutes, first 15 minutes, first 20 minutes, first 25 minutes, first 30 minutes, first 45 minutes, first hour, first 90 minutes, or first 2 hours of saccharification. In an embodiment, the peroxidase or peroxidase composition is added within the first minute, first five minutes, first 10 minutes, first 15 minutes, first 20 minutes, first 25 minutes, first 30 minutes, first 45 minutes, first hour, first 90 minutes, first 2 hours, first 3 hours, first 4 hours, first 5 hours, or first 6 hours of fermentation. In an embodiment, the peroxidase or peroxidase composition is added within the first minute, first five minutes, first 10 minutes, first 15 minutes, first 20 minutes, first 25 minutes, first 30 minutes, first 45 minutes, first hour, first 90 minutes, first 2 hours, first 3 hours, first 4 hours, first 5 hours, or first 6 hours of simultaneous saccharification and fermentation.

In an embodiment, at least one of the fermentation vessels is a yeast propagation tank and the peroxidase or peroxidase composition is introduced into the yeast propagation tank. Preferably, the peroxidase is added within the first minute, first five minutes, first 10 minutes, first 15 minutes, first 20 minutes, first 25 minutes, first 30 minutes, first 45 minutes, first hour, first 90 minutes, first 2 hours, first 3 hours, first 4 hours, first 5 hours, or first 6 hours of yeast propagation.

The peroxidase or peroxidase composition can be added to during saccharification, fermentation, simultaneous saccharification and fermentation, or yeast propagation as a single bolus, a split dose, or titrated over time within the first hour, first 90 minutes, or first 2 hours, first 3 hours, first 4 hours, first 5 hours, or first 6 hours of saccharification, fermentation, simultaneous saccharification and fermentation, or yeast propagation.

In an embodiment, the peroxidase or peroxidase composition is introduced just after liquefaction and before the fermentation tank or propagation tank. In an embodiment, the peroxidase or peroxidase composition is introduced at any point of the mash cooling system. In an embodiment, the peroxidase or peroxidase composition is added to a heat exchanger. In an embodiment, the peroxidase or peroxidase composition is added to a mixing tank.

Addition of the peroxidase or peroxidase composition to the yeast propagation tank increases growth and/or productivity of yeast during propagation compared to yeast propagated without the peroxidase. Growth and/or productivity of the yeast propagated in the presence of the peroxidase within the first hour, two hours, three hours, four hours, five hours, six hours, seven hours, eight hours, nine hours, 10 hours, 12 hours, 16 hours, 18 hours, 20 hours, 22 hours, or 24 hours of propagation is increased by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, at least 1-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 20-fold, at least 25-fold, at least 50-fold, or at least 100-fold, compared to growth and/or productivity of the yeast propagated over the same time periods without peroxidase. In an embodiment, the growth of yeast within the first 24 hours of yeast propagation is increased by from 10% to 50% compared to growth of yeast within the first 24 hours of yeast propagation without the peroxidase.

Addition of the peroxidase or peroxidase composition to the yeast propagation tank or the fermentation tank increases the amount of ethanol produced within the first 24 hours of fermentation compared to the amount of ethanol produced within the first 24 hours of fermentation when yeast propagation, saccharification, fermentation, or simultaneous saccharification and fermentation is performed without the peroxidase. In some embodiments, the amount of ethanol produced within the first hour, two hours, three hours, four hours, five hours, six hours, seven hours, eight hours, nine hours, 10 hours, 12 hours, 16 hours, 18 hours, 20 hours, 22 hours, or 24 hours of fermentation after addition of the peroxidase during yeast propagation, saccharification, fermentation, or simultaneous saccharification and fermentation is increased by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, at least 1-fold, at least 2-fold, at least 3-fold, at least 4-fold, or at least 5-fold, compared to the amount of ethanol produced over the same time period without the addition of peroxidase. In a preferred embodiment, the rate at which ethanol is produced within the first 24 hours of fermentation is increased by from 10% to 50% compared to the rate at which ethanol is produced within the first 24 hours of fermentation without the peroxidase.

Addition of the peroxidase or peroxidase composition to the yeast propagation tank or fermentation tank reduces lactic acid titers within the first 24 hours of fermentation as compared to lactic acid titers in the first 24 hours of fermentation when yeast propagation, saccharification, fermentation, or simultaneous saccharification and fermentation is performed without the peroxidase. In some embodiments, lactic acid titers within the first hour, two hours, three hours, four hours, five hours, six hours, seven hours, eight hours, nine hours, 10 hours, 12 hours, 16 hours, 18 hours, 20 hours, 22 hours, or 24 hours of fermentation are reduced by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, compared to lactic acid titers over the same period of fermentation without the addition of the peroxidase. In a preferred embodiment, titers of lactic acid within the first 24 hours of fermentation are reduced by from 10% to 50% compared to titers of lactic acid within the first 10% to 50% hours of fermentation without the peroxidase.

Addition of the peroxidase or peroxidase composition to the yeast propagation tank or fermentation tank reduces absolute titers of lactic acid at the end of fermentation compared to absolute titers of lactic acid at the end of fermentation without the addition of the peroxidase. The addition of the peroxidase to the yeast propagation tank or fermentation tank reduces absolute titers of lactic acid at the end of fermentation by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, or at least 90% compared to absolute titers of lactic acid at the end of fermentation without the addition of the peroxidase. In a preferred embodiment, absolute titers of lactic acid at the end of fermentation are reduced by from 10% to 50% compared to absolute titers of lactic acid at the end of fermentation without the peroxidase.

Yeast (e.g., a yeast composition described herein) can be added to the propagation tank or to the fermentation tank. The yeast composition introduced into the fermentation tank can comprise yeast strain described herein (e.g., Section III or Section IX). In one embodiment, the yeast composition introduced into the fermentation tank comprises a yeast strain and a peroxidase or peroxidase composition. In particular embodiments, at least one yeast composition formulated as a cream yeast, a crumbled yeast, an active dry yeast, or a compressed yeast is introduced into the fermentation tank. The at least one yeast composition formulated as a cream yeast, a crumbled yeast, an active dry yeast, or a compressed yeast can be introduced into the fermentation tank simultaneously or sequentially with the a peroxidase or peroxidase composition.

In the above embodiments, the yeast composition optionally further includes a naturally or non-naturally occurring component selected from a surfactant, emulsifier, gum, swelling aent, antioxidant, processing aid, or any combination.

Any yeast strain described herein, including yeast produced by contacting, culturing, cultivating, and/or propagating the yeast in the presence of a peroxidase, and yeast described in Section IX herein (e.g., a Saccharomyces strain, a Saccharomyces cerevisiae strain, etc.) can be used in the yeast composition.

The yeast composition can optionally be formulated to include, or introduced simultaneously or sequentially with, one or more additional enzymes. Examples of additional enzymes for formulation with, or introduction into the fermentation tank simultaneously or sequentially with, the fermenting organism composition or yeast composition include, without limitation, acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, glucan 1,4-a-glucosidase, glucan 1,4-alpha-maltohydrolase, glucan 1,4-a-glucosidase, glucan 1,4-alpha-maltohydrolase, lysophospholipase, lysozyme, alpha-mannosidase, beta-mannosidase (mannanase), phytase, phospholipase A1, phospholipase A2, phospholipase D, protease, pullulanase, pectinesterase, triacylglycerol lipase, xylanase, beta-xylosidase or any combination thereof.

In some embodiments, the yeast composition further comprises at least one, at least two, at least three, at least four, or at least five of the additional enzymes. In an embodiment, the yeast composition further comprises an alpha-amylase. In an embodiment, the yeast composition further comprises a glucoamylase. In an embodiment, the yeast composition further comprises a protease. In an embodiment, the yeast composition further comprises any combination of at least one, at least two, or all three enzymes selected from an alpha-amylase, a glucoamylase, and a protease.

In an embodiment, the yeast composition comprises a yeast strain comprising at least one, at least two, at least three, at least four, or at least five heterologous polynucleotides respectively encoding at least one, at least two, at least three, at least four, or at least five of the additional enzymes.

In a particular embodiment, the yeast composition comprises a Saccharomyces cerevisiae strain comprising at least one, at least two, or at least three heterologous polynucleotides encoding an enzyme selected from an alpha-amylase, a glucoamylase, a protease, and any combination of one, two, or all three of them.

Any yeast strain, such as especially the yeast strains described herein, for example under the heading “Fermenting organism”, can be used in the processes of propagating yeast for bioproduct production in a biofuel system. In an embodiment, the yeast belongs to a genus selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera. In an embodiment, the yeast is Saccharomyces cerevisiae, Saccharomyces pastorianus (carlsbergiensis), Kluyveromyces lactis, Kluyveromyces fragilis, Fusarium oxysporum, or any combination thereof. In an embodiment, the yeast is Saccharomyces cerevisiae.

Any peroxidase can be used in the processes of propagating yeast for bioproduct production in a biofuel system. In an embodiment, the peroxidase is a peroxidase or peroxide-decomposing enzymes selected from: E.C. 1.11.1.1 NADH peroxidase; E.C.

1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase. In an embodiment, the peroxidase is derived from a microorganism, such as a fungal organism, such a yeast or filamentous fungi, or bacteria; or plant. In an embodiment, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

The process for propagating yeast for bioproduct production in a biofuels system can be used in any biofuels system. In an embodiment, the biofuel is an alcohol. In an embodiment, the alcohol is ethanol. In an embodiment, the alcohol is methanol. In an embodiment, the alcohol is butanol.

VI. Reducing Lactic Acid and/or Preventing an Increase in Lactic Acid

In an aspect the invention relates to a process for reducing and/or preventing an increase, in lactic acid in a biofuel fermentation system, the process comprising introducing a peroxidase or peroxidase composition into a biofuel fermentation system. The peroxidase or peroxidase composition can be added at a concentration sufficient to reduce and/or prevent an increase in lactic acid in the biofuel fermentation system (e.g., an effective amount).

As used herein, the phrase “reducing and/or preventing an increase in lactic acid” encompasses the reduction of existing lactic acid present in the fermentation system, as well as preventing lactic acid levels from increasing in the system, for example due to production of lactic acid by infectious organism in the system (e.g., bacteria). For instance, a peroxidase composition or peroxidase may reduce the level of lactic acid in a fermentation system by at least 1%, 3%, 5%, 10%, 11%, 13%, 15%, 17%, 21%, 24%, 26%, 32%, 35%, 40%, 45%, 50%, 54%, 58%, 61%, 63%, 66%, 70%, 75%, 77%, 80%, 85%, 90%, 93%, 95%, 96%, 97%, 98%, 99%, or 100%.

Systems and methods for biofuel fermentation are well known in the art. The fermentation system may include one or more fermentation vessels, pipes, and/or components, which are configured to perform a fermentation product production process, such as the exemplary dry-grind ethanol production process shown in FIG. 1. Those skilled in the art will appreciate that the peroxidase or peroxidase composition may be introduced into the fermentation system at a variety of different locations. In an embodiment, at least one of the fermentation vessels in the fermentation system is a fermentation tank and the peroxidase or peroxidase composition is introduced into the fermentation tank. In an embodiment, the peroxidase or peroxidase composition is introduced to the fermentation tank before fermentation begins. In an embodiment, at least one of the fermentation vessels is a yeast propagation tank and the peroxidase or peroxidase composition is introduced into the yeast propagation tank. In an embodiment, the peroxidase or peroxidase composition is introduced just after liquefaction and before the fermentation tank or propagation tank. In an embodiment, the peroxidase or peroxidase composition is introduced at any point of the mash cooling system. In an embodiment, the peroxidase or peroxidase composition is added to a heat exchanger. In an embodiment, the peroxidase or peroxidase composition is added to a mixing tank.

In an embodiment, the biofuel is an alcohol. In an embodiment, the alcohol is ethanol. In an embodiment, the alcohol is methanol. In an embodiment, the alcohol is butanol.

### VII. Peroxidases

The present disclosure contemplates processes and compositions comprising any peroxidase, such as especially a peroxidase that enhances yeast growth and/or productivity. In an aspect, the invention relates to enhancing yeast growth and/or activity using a peroxidase. In an aspect, the invention relates to culturing, cultivating, or producing, or propagating yeast in the presence of a peroxidase. In an aspect, the invention relates to using a peroxidase in a process for propagating yeast for bioproduct production in a biofuels system. In an aspect, the invention relates to using a peroxidase in a process for producing a fermentation product, such as especially ethanol.

Any polypeptide having peroxidase activity can be used as an enzyme used in the processes of the present invention, or as a component of the enzyme composition (e.g., peroxidase composition) used in the processes of the present invention. The terms “peroxidase” and “polypeptide having peroxidase activity” are used interchangeably herein. The peroxidase may be present as an enzyme activity in the enzyme composition and/or as one or more (several) protein components added to the composition.

Examples of peroxidases are peroxidase and peroxide-decomposing enzymes including, but are not limited to, the following:

E.C. 1.11.1.1 NADH peroxidase;

E.C. 1.11.1.2 NADPH peroxidase;

E.C. 1.11.1.3 fatty-acid peroxidase;

E.C. 1.11.1.5 cytochrome-c peroxidase;

E.C. 1.11.1.6 catalase;

E.C. 1.11.1.7 peroxidase;

E.C. 1.11.1.8 iodide peroxidase;

E.C. 1.11.1.9 glutathione peroxidase;

E.C. 1.11.1.10 chloride peroxidase;

E.C. 1.11.1.11 L-ascorbate peroxidase;

E.C. 1.11.1.12 phospholipid-hydroperoxide glutathione peroxidase;

E.C. 1.11.1.13 manganese peroxidase;

E.C. 1.11.1.14 lignin peroxidase;

E.C. 1.11.1.15 peroxiredoxin;

E.C. 1.11.1.16 versatile peroxidase;

E.C. 1.11.1.B2 chloride peroxidase;

E.C. 1.11.1.B6 iodide peroxidase;

E.C. 1.11.1.B7 bromide peroxidase;

E.C. 1.11.1.B8 iodide peroxidase:

EC numbers and names can be found, e.g., at www.brenda-enzymes.org.

In one aspect, the peroxidase is an NADH peroxidase. In another aspect, the peroxidase is an NADPH peroxidase. In another aspect, the peroxidase is a fatty acid peroxidase. In another aspect, the peroxidase is a cytochrome-c peroxidase. In another aspect, the peroxidase is a catalase. In another aspect, the peroxidase is a peroxidase. In another aspect, the peroxidase is an iodide peroxidase. In another aspect, the peroxidase is a glutathione peroxidase. In another aspect, the peroxidase is a chloride peroxidase. In another aspect, the peroxidase is an L-ascorbate peroxidase. In another aspect, the peroxidase is a phospholipid-hydroperoxide glutathione peroxidase. In another aspect, the peroxidase is a manganese peroxidase. In another aspect, the peroxidase is a lignin peroxidase. In another aspect, the peroxidase is a peroxiredoxin. In another aspect, the peroxidase is a versatile peroxidase. In another aspect, the peroxidase is a chloride peroxidase. In another aspect, the peroxidase is an iodide peroxidase. In another aspect, the peroxidase is a bromide peroxidase. In another aspect, the peroxidase is an iodide peroxidase.

In a preferred embodiment the peroxidase is an E.C. 1.11.1.7 peroxidase.

Examples of peroxidases include, but are not limited to Thermoascus auranticacus peroxidase (SEQ ID NO: 1 herein) and cDNA sequence encoding Thermoascus auranticacus, Mycothermus thermophilus peroxidase (SEQ ID NO: 2 herein) and cDNA sequence encoding Mycothermus thermophilus peroxidase, and Coprinus cinereus peroxidase (Baunsgaard et al., 1993, Amino acid sequence of Coprinus macrorhizus peroxidase and cDNA sequence encoding Coprinus cinereus peroxidase. A new family of fungal peroxidases, Eur. J. Biochem. 213(1): 605-611 (Accession number P28314) or SEQ ID NO: 3 herein); horseradish peroxidase (Fujiyama et al., 1988, Structure of the horseradish peroxidase isozyme C genes, Eur. J. Biochem. 173(3): 681-687 (Accession number P15232)); peroxiredoxin (Singh and Shichi, 1998, A novel glutathione peroxidase in bovine eye. Sequence analysis, mRNA level, and translation, J. Biol. Chem. 273(40): 26171-26178 (Accession number O77834)); lactoperoxidase (Dull et al., 1990, Molecular cloning of cDNAs encoding bovine and human lactoperoxidase, DNA Cell Biol. 9(7): 499-509 (Accession number P80025)); Eosinophil peroxidase (Fornhem et al., 1996, Isolation and characterization of porcine cationic eosinophilgranule proteins, Int. Arch. Allergy Immunol. 110(2): 132-142 (Accession number P80550)); versatile peroxidase (Ruiz-Duenas et al., 1999, Molecular characterization of a novel peroxidase isolated from the ligninolytic fungus Pleurotus eryngii, Mol. Microbiol. 31(1): 223-235 (Accession number O94753)); turnip peroxidase (Mazza and Welinder, 1980, Covalent structure of turnip peroxidase 7. Cyanogen bromide fragments, complete structure and comparison to horseradish peroxidase C, Eur. J. Biochem. 108(2): 481-489 (Accession number P00434)); myeloperoxidase (Morishita et al., 1987, Chromosomal gene structure of human myeloperoxidase and regulation of its expression by granulocyte colony-stimulating factor, J. Biol. Chem. 262(31): 15208-15213 (Accession number P05164)); peroxidasin and peroxidasin homologs (Horikoshi et al., 1999, Isolation of differentially expressed cDNAs from p53-dependent apoptotic cells: activation of the human homologue of the Drosophila peroxidasin gene, Biochem. Biophys. Res. Commun. 261(3): 864-869 (Accession number Q92626)); lignin peroxidase (Tien and Tu, 1987, Cloning and sequencing of a cDNA for a ligninase from Phanerochaete chrysosporium, Nature 326(6112): 520-523 (Accession number P06181)); Manganese peroxidase (Orth et al., 1994, Characterization of a cDNA encoding a manganese peroxidase from Phanerochaete chrysosporium: genomic organization of lignin and manganese peroxidase-encoding genes, Gene 148(1): 161-165 (Accession number P78733)); alpha-dioxygenase, dual oxidase, peroxidasin, invertebrate peroxinectin, short peroxidockerin, lactoperoxidase, myeloperoxidase, non-mammalian vertebrate peroxidase, catalase, catalase-lipoxygenase fusion, di-heme cytochrome c peroxidase, methylamine utilization protein, DyP-type peroxidase, haloperoxidase, ascorbate peroxidase, catalase peroxidase, hybrid ascorbate-cytochrome c peroxidase, lignin peroxidase, manganese peroxidase, versatile peroxidase, other class II peroxidase, class III peroxidase, alkylhydroperoxidase D, other alkylhydroperoxidases, no-heme, no metal haloperoxidase, no-heme vanadium haloperoxidase, manganese catalase, NADH peroxidase, glutathione peroxidase, cysteine peroxiredoxin, thioredoxin-dependent thiol peroxidase, and AhpE-like peroxiredoxin (Passard et al., 2007, Phytochemistry 68:1605-1611).

The peroxidase activity may be obtained from microorganisms of any genus. In one aspect, the polypeptide obtained from a given source is secreted extracellularly.

The peroxidase activity may be a bacterial polypeptide. For example, the polypeptide may be a Gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having peroxidase activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having peroxidase activity.

In an embodiment the peroxidase is derived from a strain of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.

In another embodiment the peroxidase is derived from a strain of Streptococcus equisimilis, Streptococcus pyogenes, Streptococcus uberis, or Streptococcus equi subsp. Zooepidemicus.

In another aspect, the peroxidase is derived from a strain of Streptomyces achromogenes, Streptomyces avermitilis, Streptomyces coelicolor, Streptomyces griseus, or Streptomyces lividans.

The peroxidase activity may also be a fungal polypeptide, and more preferably a yeast polypeptide such as one derived from a strain of a Candida, Kluyveromyces, Pichia, Saccharomyces, Schizosaccharomyces, or Yarrowia polypeptide having peroxidase activity; or more preferably a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, lrpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria.

In another aspect, the peroxidase is derived from a strain of Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis.

In another aspect, the peroxidase is derived from a strain of Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride.

In another aspect, the peroxidase is horseradish peroxidase.

In another aspect, the peroxidase is derived from a strain of Thermoascus, such as strain of Thermoascus aurantiacus, such as the one shown in SEQ ID NO: 1 herein. In an embodiment the peroxidase has at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein. In an embodiment the peroxidase has at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to amino acids 20 to 717 of the polypeptide of SEQ ID NO: 1 herein.

In another aspect, the peroxidase is derived from a strain of Mycothermus, such as strain of Mycothermus thermophilus, such as the one shown in SEQ ID NO: 2 herein. In an embodiment the peroxidase has at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein.

In another aspect, the peroxidase is derived from a strain of Coprinus, such as Coprinus cinereus peroxidase, such as the one shown in SEQ ID NO: 3 herein. In an embodiment the peroxidase has at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein. In an embodiment the peroxidase has at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to amino acids 23 to 351 of the polypeptide of SEQ ID NO: 3 herein.

Techniques used to isolate or clone a polynucleotide encoding a polypeptide having peroxidase activity are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and nucleotide sequence-based amplification (NASBA) may be used.

### VIII. Enzyme Compositions

The present invention also relates to compositions comprising a peroxidase of the present invention. Preferably, the compositions are enriched in the a peroxidase of the invention. The term “enriched” indicates that the activity of the composition has been increased, e.g., with an enrichment factor of at least 1.1, such as at least 1.2, at least 1.3, at least 1.4, at least 1.5, at least 2.0, at least 3.0, at least 4.0, at least 5.0, at least 10.

In an embodiment, the composition comprises at least one, at least two, at least three, or at least four peroxidases of the invention.

Any peroxidase can be used a composition of the present invention (e.g., peroxidase composition). In an embodiment, the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase. In an embodiment, the peroxidase is derived from a microorganism, such as a fungal organism, such a yeast or filamentous fungi, or bacteria; or plant. In an embodiment, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

The compositions may further comprise multiple enzymatic activities, such as one or more (e.g., several) enzymes selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, glucan 1,4-a-glucosidase, glucan 1,4-alpha-maltohydrolase, glucan 1,4-a-glucosidase, glucan 1,4-alpha-maltohydrolase, lysophospholipase, lysozyme, alpha-mannosidase, beta-mannosidase (mannanase), phytase, phospholipase A1, phospholipase A2, phospholipase D, protease, pullulanase, pectinesterase, triacylglycerol lipase, xylanase, beta-xylosidase or any combination thereof. In an embodiment, the composition comprises a peroxidase and at least one, at least two, at least three, at least four, or at least five of the additional enzymatic activities. In an embodiment, the composition comprises at least two peroxidases and at least one, at least two, at least three, at least four, or at least five of the additional enzymatic activities. In an embodiment, the composition comprises at least three peroxidases and at least one, at least two, at least three, at least four, or at least five of the additional enzymatic activities. In an embodiment, the composition comprises at least four peroxidases and at least one, at least two, at least three, at least four, or at least five of the additional enzymatic activities.

In an embodiment, the composition comprises a peroxidase of the invention and a glucoamylase. In an embodiment, the composition comprises a peroxidase of the invention and a glucoamylase derived from Talaromyces emersonii (e.g., SEQ ID NO: 4) or a variant thereof. In an embodiment the composition comprises a peroxidase of the invention and a glucoamylase derived from Gloeophyllum, such as G. serpiarium (e.g., SEQ ID NO: 5) or G. trabeum (e.g., SEQ ID NO: 6) or variants thereof. In an embodiment the composition comprises a peroxidase of the invention and a glucoamylase derived from the genus Pycnoporus, in particular a strain of Pycnoporus as described in WO 2011/066576 (SEQ ID NO: 2, 4 or 6 therein), including the Pycnoporus sanguineus glucoamylase having SEQ ID NO: 7 herein or a variant thereof. In an embodiment the composition comprises a peroxidase of the invention and a glucoamylase derived from Triametes, in such as Triametes cingulate glucoamylase having SEQ ID NO: 8 herein or a variant thereof.

In an embodiment the composition comprises a peroxidase of the invention, a glucoamylase and an alpha-amylase. In an embodiment the composition comprises a peroxidase of the invention, a glucoamylase and an alpha-amylase derived from Rhizomucor, preferably a strain the Rhizomucor pusillus, such as a Rhizomucor pusillus alpha-amylase hybrid having an linker (e.g., from Aspergillus niger) and starch-bonding domain (e.g., from Aspergillus niger). In an embodiment the composition comprises a peroxidase of the invention, a glucoamylase, an alpha-amylase and a cellulolytic enzyme composition. In an embodiment the composition comprises a peroxidase of the invention, a glucoamylase, an alpha-amylase and a cellulolytic enzyme composition, wherein the cellulolytic composition is derived from Trichoderma reesei. In an embodiment the composition comprises a peroxidase of the invention, a glucoamylase, an alpha-amylase and a protease. In an embodiment the composition comprises a peroxidase of the invention, a glucoamylase, an alpha-amylase, a protease, and a trehalase. The protease may be derived from Thermoascus aurantiacus. In an embodiment the composition comprises a peroxidase of the invention, a glucoamylase, an alpha-amylase, a cellulolytic enzyme composition and a protease. In an embodiment the composition comprises a peroxidase of the invention, a glucoamylase, an alpha-amylase, a cellulolytic enzyme composition, a protease, and a trehalase. In an embodiment the composition comprises a peroxidase of the invention, a glucoamylase, e.g., derived from Talaromyces emersonii, Gloeophyllum serpiarium or Gloephyllum trabeum, an alpha-amylase, e.g., derived from Rhizomucor pusillus, in particular one having a linker and starch-binding domain, in particular derived from Aspergillus niger, in particular one having the following substitutions: G128D+D143N (using SEQ ID NO: 9 for numbering); a cellulolytic enzyme composition derived from Trichoderma reesei, and a protease, e.g., derived from Thermoascus aurantiacus or Meripilus giganteus. In an embodiment the composition comprises a peroxidase of the invention, a glucoamylase, e.g., derived from Talaromyces emersonii, Gloeophyllum serpiarium or Gloephyllum trabeum, an alpha-amylase, e.g., derived from Rhizomucor pusillus, in particular one having a linker and starch-binding domain, in particular derived from Aspergillus niger, in particular one having the following substitutions: G128D+D143N (using SEQ ID NO: 9 for numbering); a cellulolytic enzyme composition derived from Trichoderma reesei, and a protease, e.g., derived from Thermoascus aurantiacus or Meripilus giganteus, and a trehalase.

Any trehalase can be used in the compositions and processes of the invention. In an embodiment, the trehalase is derived from a strain of Talaromyces, such as strain of Talaromyces funiculosus, such as the one shown in SEQ ID NO: 28 herein, or one having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 28 herein, or a strain of Talaromyces leycettanus such as the one shown in SEQ ID NO: 29 herein, or one having at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO: 29 herein.

The compositions may be prepared in accordance with methods known in the art and may be in the form of a liquid or a dry composition. The compositions may be stabilized in accordance with methods known in the art. In an embodiment, the composition comprises one or more formulating agents as disclosed herein, preferably one or more of the compounds selected from the list consisting of glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose.

In an embodiment, the composition comprises one or more components selected from the list consisting of vitamins, minerals and amino acids.

Examples are given below of preferred uses of the compositions of the present invention. The dosage of the composition and other conditions under which the composition is used may be determined on the basis of methods known in the art.

### IX. Processes for Producing Fermentation Products

The invention also relates to processes for producing a fermentation product from starch-containing material using a fermenting organism, wherein a peroxidase or an enzyme composition comprising a peroxidase is added before and/or during saccharification and/or fermentation.

Processes for Producing Fermentation Products from Un-Gelatinized Starch-Containing Material

In an aspect, the invention relates to processes for producing fermentation products from starch-containing material without gelatinization (i.e., without cooking) of the starch-containing material (often referred to as a “raw starch hydrolysis” process), wherein a peroxidase is added. The fermentation product, such as ethanol, can be produced without liquefying the aqueous slurry containing the starch-containing material and water. In one embodiment a process of the invention includes saccharifying (e.g., milled) starch-containing material, e.g., granular starch, below the initial gelatinization temperature, preferably in the presence of alpha-amylase and/or carbohydrate-source generating enzyme(s) to produce sugars that can be fermented into the fermentation product by a suitable fermenting organism. In this embodiment the desired fermentation product, e.g., ethanol, is produced from un-gelatinized (i.e., uncooked), preferably milled, cereal grains, such as corn.

Accordingly, in one aspect the invention relates to processes for producing a fermentation product from starch-containing material comprising simultaneously saccharifying and fermenting starch-containing material using a carbohydrate-source generating enzymes and a fermenting organism at a temperature below the initial gelatinization temperature of said starch-containing material in the presence of a variant protease of the invention. Saccharification and fermentation may also be separate. Thus in another aspect the invention relates to processes of producing fermentation products, comprising the following steps:

(b) saccharifying a starch-containing material at a temperature below the initial gelatinization temperature using a carbohydrate-source generating enzyme, e.g., a glucoamylase; and
(c) fermenting using a fermentation organism;
wherein step (b) and/or (c) is carried out using at least a glucoamylase and a peroxidase or peroxidase composition of the invention. In an embodiment, said peroxidase or peroxidase composition is added at a concentration sufficient to enhance growth and/or productivity of yeast. Note that step (a) was intentionally omitted from this raw starch process so that the saccharification step (b) and fermentation step (c) of the raw starch process correspond to saccharification step (b) and fermentation step (c) of the convention process described below, which includes a liquefaction step (a).

In an embodiment, the a peroxidase or peroxidase composition is added during saccharifying step (b). Preferably, the peroxidase or peroxidase composition is added within the first minute, first five minutes, first 10 minutes, first 15 minutes, first 20 minutes, first 25 minutes, first 30 minutes, first 45 minutes, first hour, first 90 minutes, or first 2 hours of saccharification. In an embodiment, the peroxidase or peroxidase composition is added within the first hour of saccharification. In an embodiment, the peroxidase or peroxidase composition is added within the 90 minutes of saccharification. In an embodiment, the a peroxidase or peroxidase composition is added during fermenting step (c). Preferably, the peroxidase is added within the first minute, first five minutes, first 10 minutes, first 15 minutes, first 20 minutes, first 25 minutes, first 30 minutes, first 45 minutes, first hour, first 90 minutes, first 2 hours, first 3 hours, first 4 hours, first 5 hours, or first 6 hours of fermentation.

In one embodiment, an alpha amylase, in particular a fungal alpha-amylase, is also added in step (b). Steps (b) and (c) may be performed simultaneously. In an embodiment, the a peroxidase is added during simultaneous saccharification and fermentation (SSF). Preferably, the peroxidase is added within the first minute, first five minutes, first 10 minutes, first 15 minutes, first 20 minutes, first 25 minutes, first 30 minutes, first 45 minutes, first hour, first 90 minutes, first 2 hours, first 3 hours, first 4 hours, first 5 hours, or first 6 hours of simultaneous saccharification and fermentation.

In an embodiment, the process further includes propagating a fermenting organism under conditions suitable to be further used in fermentation. In an embodiment, the fermenting organism is yeast and the peroxidase or peroxidase composition is added during yeast propagation. Preferably, the peroxidase or peroxidase composition is added within the first minute, first five minutes, first 10 minutes, first 15 minutes, first 20 minutes, first 25 minutes, first 30 minutes, first 45 minutes, first hour, first 90 minutes, first 2 hours, first 3 hours, first 4 hours, first 5 hours, or first 6 hours of yeast propagation.

The peroxidase or peroxidase composition can be added to during saccharification, fermentation, simultaneous saccharification and fermentation, or yeast propagation as a single bolus, a split dose, or titrated over time within the first hour, first 90 minutes, first 2 hours, first 3 hours, first 4 hours, first 5 hours, or first 6 hours of saccharification, fermentation, simultaneous saccharification and fermentation, or yeast propagation.

Addition of the peroxidase or peroxidase composition during yeast propagation increases growth and/or productivity of yeast during propagation compared to yeast propagated without the peroxidase or peroxidase composition. Growth and/or productivity of the yeast propagated in the presence of the peroxidase or peroxidase composition within the first hour, two hours, three hours, four hours, five hours, six hours, seven hours, eight hours, nine hours, 10 hours, 12 hours, 16 hours, 18 hours, 20 hours, 22 hours, or 24 hours of propagation is increased by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, at least 1-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 20-fold, at least 25-fold, at least 50-fold, or at least 100-fold, compared to growth and/or productivity of the yeast propagated over the same time periods without peroxidase or peroxidase composition. In an embodiment, the growth of yeast within the first 24 hours of yeast propagation is increased by from 10% to 50% compared to growth of yeast within the first 24 hours of yeast propagation without the peroxidase or peroxidase composition.

Addition of the peroxidase or peroxidase composition during yeast propagation, saccharification, fermentation, or simultaneous saccharification and fermentation increases the amount of ethanol produced within the first 24 hours of fermentation compared to the amount of ethanol produced within the first 24 hours of fermentation when yeast propagation, saccharification, fermentation, or simultaneous saccharification and fermentation is performed without the peroxidase or peroxidase composition. In some embodiments, the rate at which ethanol is produced within the first hour, two hours, three hours, four hours, five hours, six hours, seven hours, eight hours, nine hours, 10 hours, 12 hours, 16 hours, 18 hours, 20 hours, 22 hours, or 24 hours of fermentation after addition of the peroxidase during yeast propagation, saccharification, fermentation, or simultaneous saccharification and fermentation is increased by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, at least 1-fold, at least 2-fold, at least 3-fold, at least 4-fold, or at least 5-fold, compared to the rate at which ethanol is produced over the same time period without the addition of a peroxidase or peroxidase composition. In a preferred embodiment, the rate at which ethanol is produced within the first 24 hours of fermentation is increased by from 10% to 50% compared to the amount of ethanol produced within the first 24 hours of fermentation without the peroxidase or peroxidase composition.

Addition of the peroxidase or peroxidase composition during yeast propagation, saccharification, fermentation, or simultaneous saccharification and fermentation reduces lactic acid titers within the first 24 hours of fermentation as compared to lactic acid titers in the first 24 hours of fermentation when yeast propagation, saccharification, fermentation, or simultaneous saccharification and fermentation is performed without the peroxidase or peroxidase composition. In some embodiments, lactic acid titers within the first hour, two hours, three hours, four hours, five hours, six hours, seven hours, eight hours, nine hours, 10 hours, 12 hours, 16 hours, 18 hours, 20 hours, 22 hours, or 24 hours of fermentation are reduced by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, compared to lactic acid titers over the same period of fermentation without the addition of the peroxidase or peroxidase composition. In a preferred embodiment, titers of lactic acid within the first 24 hours of fermentation are reduced by from 10% to 50% compared to titers of lactic acid within the first 24 hours of fermentation without the peroxidase or peroxidase composition.

Addition of the peroxidase or peroxidase composition during yeast propagation, saccharification, fermentation, or simultaneous saccharification and fermentation reduces absolute titers of lactic acid at the end of fermentation compared to absolute titers of lactic acid at the end of fermentation without the addition of the peroxidase or peroxidase composition. The addition of the peroxidase during yeast propagation, saccharification, fermentation, or simultaneous saccharification and fermentation reduces absolute titers of lactic acid at the end of fermentation by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, or at least 90% compared to absolute titers of lactic acid at the end of fermentation without the addition of the peroxidase or peroxidase composition. In a preferred embodiment, absolute titers of lactic acid at the end of fermentation are reduced by from 10% to 50% compared to absolute titers of lactic acid at the end of fermentation without the peroxidase or peroxidase composition.

Any yeast strain, such as especially the yeast strains described herein, for example under the heading “Fermenting organism”, can be used as the fermenting organism in the processes for producing a fermentation product from starch-containing material. In an embodiment, the yeast belongs to a genus selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera. In an embodiment, the yeast is Saccharomyces cerevisiae, Saccharomyces pastorianus (carlsbergiensis), Kluyveromyces lactis, Kluyveromyces fragilis, Fusarium oxysporum, or any combination thereof. In an embodiment, the yeast is Saccharomyces cerevisiae.

Any peroxidase can be used in the processes for producing a fermentation product from starch-containing material. In an embodiment, the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase. In an embodiment, the peroxidase is derived from a microorganism, such as a fungal organism, such a yeast or filamentous fungi, or bacteria; or plant. In an embodiment, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

Processes for Producing Fermentation Products from Gelatinized Starch-Containing Material

In an aspect, the invention relates to processes for producing fermentation products, especially ethanol, from starch-containing material, which process includes a liquefaction step and sequentially or simultaneously performed saccharification and fermentation steps. Consequently, the invention relates to a process for producing a fermentation product from starch-containing material comprising the steps of:

(a) liquefying starch-containing material in the presence of an alpha-amylase to form a liquefied mash;
(b) saccharifying the liquefied mash using a carbohydrate-source generating enzyme to produce a fermentable sugar; and
(c) fermenting the sugar using a fermenting organism under conditions suitable to produce the fermentation product;
wherein a peroxidase or peroxidase composition is added before or during saccharifying step (b) and/or fermenting step (c). In an embodiment, said peroxidase or peroxidase composition is added at a concentration sufficient to enhance growth and/or productivity of yeast.

In an embodiment, the peroxidase or peroxidase composition is added before or during saccharifying step (b). Preferably, the peroxidase or peroxidase composition is added within the first minute, first five minutes, first 10 minutes, first 15 minutes, first 20 minutes, first 25 minutes, first 30 minutes, first 45 minutes, first hour, first 90 minutes, or first 2 hours of saccharification. In an embodiment, the a peroxidase or peroxidase composition is added before or during fermenting step (c). Preferably, the peroxidase or peroxidase composition is added within the first minute, first five minutes, first 10 minutes, first 15 minutes, first 20 minutes, first 25 minutes, first 30 minutes, first 45 minutes, or first hour, first 90 minutes, first 2 hours, first 3 hours, first 4 hours, first 5 hours, or first 6 hours of fermentation.

In one embodiment, an alpha amylase, in particular a fungal alpha-amylase, is also added in step (b). Steps (b) and (c) may be performed simultaneously. In an embodiment, the a peroxidase is added during simultaneous saccharification and fermentation (SSF). Preferably, the peroxidase or peroxidase composition is added within the first minute, first five minutes, first 10 minutes, first 15 minutes, first 20 minutes, first 25 minutes, first 30 minutes, first 45 minutes, or first hour, first 90 minutes, first 2 hours, first 3 hours, first 4 hours, first 5 hours, or first 6 hours of simultaneous saccharification and fermentation.

In an embodiment, the process further includes propagating a fermenting organism under conditions suitable to be further used in fermentation. In an embodiment, the fermenting organism is yeast and the a peroxidase or peroxidase composition is added before or during yeast propagation. Preferably, the peroxidase or peroxidase composition is added within the first minute, first five minutes, first 10 minutes, first 15 minutes, first 20 minutes, first 25 minutes, first 30 minutes, first 45 minutes, or first hour, first 90 minutes, first 2 hours, first 3 hours, first 4 hours, first 5 hours, or first 6 hours of yeast propagation. In an embodiment, the peroxidase or peroxidase composition is added within the first 4 hours of yeast propagation. In an embodiment, the peroxidase or peroxidase composition is added within the first 6 hours of yeast propagation.

The peroxidase or peroxidase composition can be added to during saccharification, fermentation, simultaneous saccharification and fermentation, or yeast propagation as a single bolus, a split dose, or titrated over time within the first hour, first 90 minutes, first 2 hours, first 3 hours, first 4 hours, first 5 hours, or first 6 hours of saccharification, fermentation, simultaneous saccharification and fermentation, or yeast propagation.

Addition of the peroxidase or peroxidase composition during yeast propagation increases growth and/or productivity of yeast during propagation compared to yeast propagated without the peroxidase or peroxidase composition. Growth and/or productivity of the yeast propagated in the presence of the peroxidase or peroxidase composition within the first hour, two hours, three hours, four hours, five hours, six hours, seven hours, eight hours, nine hours, 10 hours, 12 hours, 16 hours, 18 hours, 20 hours, 22 hours, or 24 hours of propagation is increased by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, at least 1-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 20-fold, at least 25-fold, at least 50-fold, or at least 100-fold, compared to growth and/or productivity of the yeast propagated over the same time periods without peroxidase or peroxidase composition. In an embodiment, the growth of yeast within the first 24 hours of yeast propagation is increased by from 10% to 50% compared to growth of yeast within the first 24 hours of yeast propagation without the peroxidase or peroxidase composition.

Addition of the peroxidase or peroxidase composition during yeast propagation, saccharification, fermentation, or simultaneous saccharification and fermentation increases the amount of ethanol produced within the first 24 hours of fermentation compared to the amount of ethanol produced within the first 24 hours of fermentation when yeast propagation, saccharification, fermentation, or simultaneous saccharification and fermentation is performed without the peroxidase or peroxidase composition. In some embodiments, the amount of ethanol produced within the first hour, two hours, three hours, four hours, five hours, six hours, seven hours, eight hours, nine hours, 10 hours, 12 hours, 16 hours, 18 hours, 20 hours, 22 hours, or 24 hours of fermentation after addition of the peroxidase during yeast propagation, saccharification, fermentation, or simultaneous saccharification and fermentation is increased by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, at least 1-fold, at least 2-fold, at least 3-fold, at least 4-fold, or at least 5-fold, compared to the amount of ethanol produced over the same time period without the addition of peroxidase or peroxidase composition. In a preferred embodiment, the rate at which ethanol is produced within the first 24 hours of fermentation is increased by from 10% to 50% compared to the amount of ethanol produced within the first 24 hours of fermentation without the peroxidase or peroxidase composition.

Addition of the peroxidase or peroxidase composition during yeast propagation, saccharification, fermentation, or simultaneous saccharification and fermentation reduces lactic acid titers within the first 24 hours of fermentation as compared to lactic acid titers in the first 24 hours of fermentation when yeast propagation, saccharification, fermentation, or simultaneous saccharification and fermentation is performed without the peroxidase or peroxidase composition. In some embodiments, lactic acid titers within the first hour, two hours, three hours, four hours, five hours, six hours, seven hours, eight hours, nine hours, 10 hours, 12 hours, 16 hours, 18 hours, 20 hours, 22 hours, or 24 hours of fermentation are reduced by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, at least 90%, compared to lactic acid titers over the same period of fermentation without the addition of the peroxidase or peroxidase composition. In a preferred embodiment, titers of lactic acid within the first 24 hours of fermentation are reduced by from 10% to 50% compared to titers of lactic acid within the first 24 hours of fermentation without the peroxidase or peroxidase composition.

Addition of the peroxidase or peroxidase composition during yeast propagation, saccharification, fermentation, or simultaneous saccharification and fermentation reduces absolute titers of lactic acid at the end of fermentation compared to absolute titers of lactic acid at the end of fermentation without the addition of the peroxidase or peroxidase composition. The addition of the peroxidase or peroxidase composition during yeast propagation, saccharification, fermentation, or simultaneous saccharification and fermentation reduces absolute titers of lactic acid at the end of fermentation by at least 3%, at least 5%, at least 7%, at least 10%, at least 12%, at least 15%, at least 25%, at least 30%, at least 33%, at least 40%, at least 50%, at least 66%, at least 75%, at least 80%, at least 85%, or at least 90% compared to absolute titers of lactic acid at the end of fermentation without the addition of the peroxidase or peroxidase composition. In a preferred embodiment, absolute titers of lactic acid at the end of fermentation are reduced by from 10% to 50% compared to absolute titers of lactic acid at the end of fermentation without the peroxidase or peroxidase composition.

Any yeast strain, such as especially the yeast strains described herein, for example under the heading “Fermenting organism”, can be used as the fermenting organism in the processes for producing a fermentation product from starch-containing material. In an embodiment, the yeast belongs to a genus selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera. In an embodiment, the yeast is Saccharomyces cerevisiae, Saccharomyces pastorianus (carlsbergiensis), Kluyveromyces lactis, Kluyveromyces fragilis, Fusarium oxysporum, or any combination thereof. In an embodiment, the yeast is Saccharomyces cerevisiae.

Any peroxidase can be used in the processes for producing a fermentation product from starch-containing material. In an embodiment, the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase. In an embodiment, the peroxidase is derived from a microorganism, such as a fungal organism, such a yeast or filamentous fungi, or bacteria; or plant. In an embodiment, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

The slurry is heated to above the gelatinization temperature and an alpha-amylase variant may be added to initiate liquefaction (thinning). The slurry may in an embodiment be jet-cooked to further gelatinize the slurry before being subjected to alpha-amylase in step (a). Liquefaction may in an embodiment be carried out as a three-step hot slurry process. The slurry is heated to between 60-95° C., preferably between 70-90° C., such as preferably between 80-85° C. at a pH of 4-6, in particular at a pH of 4.5-5.5, and alpha-amylase variant, optionally together with a hemicellulase, an endoglucanase, a protease, a carbohydrate-source generating enzyme, such as a glucoamylase, a phospholipase, a phytase, and/or pullulanase, are added to initiate liquefaction (thinning). The liquefaction process is usually carried out at a pH of 4-6, in particular at a pH from 4.5 to 5.5. Saccharification step (b) may be carried out using conditions well known in the art. For instance, a full saccharification process may last up to from about 24 to about 72 hours, however, it is common only to do a pre-saccharification of typically 40-90 minutes at a temperature between 30-65° C., typically about 60° C., followed by complete saccharification during fermentation in a simultaneous saccharification and fermentation process (SSF process). Saccharification is typically carried out at a temperature from 20-75° C., in particular 40-70° C., typically around 60° C., and at a pH between 4 and 5, normally at about pH 4.5. The most widely used process to produce a fermentation product, especially ethanol, is a simultaneous saccharification and fermentation (SSF) process, in which there is no holding stage for the saccharification, meaning that a fermenting organism, such as yeast, and enzyme(s), may be added together. SSF may typically be carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours.

### Starch-Containing Materials

According to the invention any suitable starch-containing starting material may be used. The starting material is generally selected based on the desired fermentation product, in particular ethanol. Examples of starch-containing starting materials, suitable for use in processes of the present invention, include cereal, tubers or grains. Specifically the starch-containing material may be corn, wheat, barley, rye, milo, sago, cassava, tapioca, sorghum, oat, rice, peas, beans, or sweet potatoes, or mixtures thereof. Contemplated are also waxy and non-waxy types of corn and barley.

In a preferred embodiment the starch-containing starting material is corn.

In a preferred embodiment the starch-containing starting material is wheat.

In a preferred embodiment the starch-containing starting material is barley.

In a preferred embodiment the starch-containing starting material is rye.

In a preferred embodiment the starch-containing starting material is milo.

In a preferred embodiment the starch-containing starting material is sago.

In a preferred embodiment the starch-containing starting material is cassava.

In a preferred embodiment the starch-containing starting material is tapioca.

In a preferred embodiment the starch-containing starting material is sorghum.

In a preferred embodiment the starch-containing starting material is rice,

In a preferred embodiment the starch-containing starting material is peas.

In a preferred embodiment the starch-containing starting material is beans.

In a preferred embodiment the starch-containing starting material is sweet potatoes.

In a preferred embodiment the starch-containing starting material is oats.

### Fermentation Products

The term “fermentation product” means a product produced by a method or process including fermenting using a fermenting organism. A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol);

an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); antibiotics (e.g., penicillin and tetracycline); enzymes; a gas (e.g., methane, hydrogen (H₂), carbon dioxide (CO₂), and carbon monoxide (CO)); isoprene; a ketone (e.g., acetone); hormones; an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); polyketide; and vitamins (e.g., riboflavin, B₁₂, beta-carotene).

In one aspect, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. In an embodiment the fermentation product is ethanol.

In another aspect, the fermentation product is an alkane. The alkane may be an unbranched or a branched alkane. The alkane can be, but is not limited to, pentane, hexane, heptane, octane, nonane, decane, undecane, or dodecane.

In another aspect, the fermentation product is a cycloalkane. The cycloalkane can be, but is not limited to, cyclopentane, cyclohexane, cycloheptane, or cyclooctane.

In another aspect, the fermentation product is an alkene. The alkene may be an unbranched or a branched alkene. The alkene can be, but is not limited to, pentene, hexene, heptene, or octene.

In another aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another aspect, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another aspect, the fermentation product is isoprene.

In another aspect, the fermentation product is a ketone. The term “ketone” encompasses a substance that contains one or more ketone moieties. The ketone can be, but is not limited to, acetone.

In another aspect, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another aspect, the fermentation product is polyketide.

### Fermenting Organisms

The fermenting organism described herein may be derived from any host cell known to the skilled artisan capable of producing a fermentation product, such as ethanol. As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

Examples of fermenting organisms include fungal organisms such as yeast. Preferred yeast include strains of Saccharomyces, in particular Saccharomyces cerevisiae or Saccharomyces uvarum; strains of Pichia, in particular Pichia stipitis such as Pichia stipitis CBS 5773 or Pichia pastoris; strains of Candida, in particular Candida arabinofermentans, Candida boidinii, Candida diddensii, Candida shehatae, Candida sonorensis, Candida tropicalis, or Candida utilis. Other fermenting organisms include strains of Hansenula, in particular Hansenula anomala or Hansenula polymorpha; strains of Kluyveromyces, in particular Kluyveromyces fragilis or Kluyveromyces marxianus; and strains of Schizosaccharomyces, in particular Schizosaccharomyces pombe.

In an embodiment, the fermenting organism is a C6 sugar fermenting organism, such as a strain of, e.g., Saccharomyces cerevisiae.

In an embodiment, the fermenting organism is a C5 sugar fermenting organism, such as a strain of, e.g., Saccharomyces cerevisiae.

The host cells for preparing the recombinant cells described herein can be from any suitable host, such as a yeast strain, including, but not limited to, a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell. In particular, Saccharomyces host cells are contemplated, such as Saccharomyces cerevisiae, bayanus or carlsbergensis cells. Preferably, the yeast cell is a Saccharomyces cerevisiae cell. Suitable cells can, for example, be derived from commercially available strains and polyploid or aneuploid industrial strains, including but not limited to those from Superstart™, THERMOSACC®, C5 FUEL™, XyloFerm®, etc. (Lallemand); RED STAR and ETHANOL RED® (Fermentis/Lesaffre); FALI (AB Mauri); Baker's Best Yeast, Baker's Compressed Yeast, etc. (Fleishmann's Yeast); BIOFERM AFT, XP, CF, and XR (North American Bioproducts Corp.); Turbo Yeast (Gert Strand AB); and FERMIOL® (DSM Specialties). Other useful yeast strains are available from biological depositories such as the American Type Culture Collection (ATCC) or the Deutsche Sammlung von Mikroorganismen and Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, 5150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The fermenting organism may be Saccharomyces strain, e.g., Saccharomyces cerevisiae strain produced using the method described and concerned in U.S. Pat. No. 8,257,959-BB.

The strain may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference) or any strain described in PCT/US2016/061887 (incorporated herein by reference).

The fermenting organisms according to the invention have been generated in order to improve fermentation yield and to improve process economy by cutting enzyme costs since part or all of the necessary enzymes needed to improve method performance are be produced by the fermenting organism.

The fermenting organisms described herein may utilize expression vectors comprising the coding sequence of one or more (e.g., two, several) heterologous genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

Aspects of the invention relate to fermenting organisms comprising heterologous polynucleotides encoding enzymes used in saccharification, fermentation, and/or simultaneous saccharification and fermentation. Examples of suitable enzymes include, without limitation, acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, glucan 1,4-a-glucosidase, glucan 1,4-alpha-maltohydrolase, glucan 1,4-a-glucosidase, glucan 1,4-alpha-maltohydrolase, lysophospholipase, lysozyme, alpha-mannosidase, beta-mannosidase (mannanase), phytase, phospholipase A1, phospholipase A2, phospholipase D, protease, pullulanase, pectinesterase, triacylglycerol lipase, xylanase, beta-xylosidase or any combination thereof.

In an embodiment, the fermenting organism comprising a heterologous polynucleotide encoding an enzyme selected from an alpha-amylase, a cellulase, a glucoamylase, a protease, a trehalase, and any combination thereof. In an embodiment, the fermenting organism is a yeast strain comprising a heterologous polynucleotide encoding an enzyme selected from an alpha-amylase, a cellulase, a glucoamylase, a protease, a trehalase, and any combination thereof. In an embodiment, the fermenting organism is a Saccharomyces yeast strain comprising a heterologous polynucleotide encoding an enzyme selected from an alpha-amylase, a cellulase, a glucoamylase, a protease, and any combination thereof. In an embodiment, the fermenting organism is a Saccharomyces cerevisiae yeast strain comprising a heterologous polynucleotide encoding an enzyme selected from an alpha-amylase, a cellulase, a glucoamylase, a protease, a trehalase, and any combination thereof.

In an embodiment, the fermenting organism, e.g., yeast, e.g., a Saccharomyces strain, such as a Saccharomyces cerevisiae strain, comprises a heterologous polynucleotide encoding a alpha-amylase.

In one embodiment, the bacterial alpha-amylase is derived from an alpha-amylase described in U.S. Application No. 62/514,636, filed Jun. 2, 2017 (Attorney Docket No. 14480-US-PRO, which is incorporated herein in its entirety) selected from the Bacillus subtilis alpha-amylase of SEQ ID NO: 76 therein, the Bacillus subtilis alpha-amylase of SEQ ID NO: 82 therein, the Bacillus subtilis alpha-amylase of SEQ ID NO: 83 therein, the Bacillus subtilis alpha-amylase of SEQ ID NO: 84 therein, or the Bacillus licheniformis alpha-amylase of SEQ ID NO: 85 therein, the Clostridium phytofermentans alpha-amylase of SEQ ID NO: 89 therein, the Clostridium phytofermentans alpha-amylase of SEQ ID NO: 90 therein, the Clostridium phytofermentans alpha-amylase of SEQ ID NO: 91 therein, the Clostridium phytofermentans alpha-amylase of SEQ ID NO: 92 therein, the Clostridium phytofermentans alpha-amylase of SEQ ID NO: 93 therein, the Clostridium phytofermentans alpha-amylase of SEQ ID NO: 94 therein, the Clostridium thermocellum alpha-amylase of SEQ ID NO: 10 therein, the Thermobifida fusca alpha-amylase of SEQ ID NO: 11 therein, the Thermobifida fusca alpha-amylase of SEQ ID NO: 97 therein, the Anaerocellum thermophilum of SEQ ID NO: 98 therein, the Anaerocellum thermophilum of SEQ ID NO: 99 therein, the Anaerocellum thermophilum of SEQ ID NO: 100 therein, the Streptomyces avermitilis of SEQ ID NO: 101 therein, or the Streptomyces avermitilis of SEQ ID NO: 88 therein.

In one embodiment, the alpha-amylase is derived from a yeast alpha-amylase, such as a yeast alpha-amylase described in U.S. Application No. 62/514,636 filed Jun. 2, 2017 (Attorney Docket No. 14480-US-PRO, which is incorporated herein in its entirety) selected from the Saccharomycopsis fibuligera alpha-amylase of SEQ ID NO: 77 therein, the Debaryomyces occidentalis alpha-amylase of SEQ ID NO: 78 therein, the Debaryomyces occidentalis alpha-amylase of SEQ ID NO: 79 therein, the Lipomyces kononenkoae alpha-amylase of SEQ ID NO: 80 therein, the Lipomyces kononenkoae alpha-amylase of SEQ ID NO: 81 therein.

In one embodiment, the alpha-amylase is derived from a filamentous fungal alpha-amylase, such as a filamentous fungal alpha-amylase described in U.S. Application No. 62/514,636 filed Jun. 2, 2017 (Attorney Docket No. 14480-US-PRO, which is incorporated herein in its entirety) selected from the Aspergillus niger alpha-amylase of SEQ ID NO: 86 therein, or the Aspergillus niger alpha-amylase of SEQ ID NO: 87 therein.

Additional alpha-amylases contemplated for use with the present invention can be found in WO2011/153516 (the content of which is incorporated herein).

Additional polynucleotides encoding suitable alpha-amylases may be obtained from microorganisms of any genus, including those readily available within the UniProtKB database (www.uniprot.org).

The alpha-amylase coding sequences can also be used to design nucleic acid probes to identify and clone DNA encoding alpha-amylases from strains of different genera or species, as described supra.

The polynucleotides encoding alpha-amylases may also be identified and obtained from other sources including microorganisms isolated from nature (e.g., soil, composts, water, etc.) or DNA samples obtained directly from natural materials (e.g., soil, composts, water, etc.,) as described supra.

Techniques used to isolate or clone polynucleotides encoding alpha-amylases are described supra.

In an embodiment, the fermenting organism, e.g., yeast, e.g., a Saccharomyces strain, such as a Saccharomyces cerevisiae strain, comprises a heterologous polynucleotide encoding a glucoamylase.

In an embodiment, the fermenting organism, e.g., yeast, e.g., a Saccharomyces strain, such as a Saccharomyces cerevisiae strain, comprises a heterologous polynucleotide encoding a protease. Exemplary proteases that may be expressed with the fermenting organism, e.g., yeast, e.g., a Saccharomyces strain, such as a Saccharomyces cerevisiae strain and processes described herein include, without limitation, the proteases shown in Table 1 of U.S. Application No. 62/514,636, filed Jun. 2, 2017 (Attorney Docket No. 14480-US-PRO), which is incorporated by reference herein in its entirety, i.e., at least one, at least two, at least three, at least four, or at least five of any of SEQ ID Nos: 9-73 therein (or variants thereof having at least 60%, at least 65%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto).

A construct or vector (or multiple constructs or vectors) comprising the one or more (e.g., two, several) heterologous genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one embodiment, the heterologous polynucleotide encoding the hexose transporter is operably linked to a promoter foreign to the polynucleotide. The promoters may be identical to or share a high degree of sequence identity (e.g., at least about 80%, at least about 85%, at least about 90%, at least about 95%, or at least about 99%) with a selected native promoter.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, xylose reductase (XR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5′-phosphate decarboxylase (URA3) genes. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, or at least about 99%) with the selected native terminator.

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL10 terminator). Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15: 5983-5990.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo. Examples of origins of replication for use in a yeast host cell are the 2 micron origin of replication, ARS1, ARS4, the combination of ARS1 and CEN3, and the combination of ARS4 and CEN6.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, supra).

Additional procedures and techniques known in the art for the preparation of recombinant cells for ethanol fermentation, are described in, e.g., WO 2016/045569, the content of which is hereby incorporated by reference.

The fermenting organism may be in the form of a composition comprising a fermenting organism (e.g., a yeast strain described herein) and a naturally occurring and/or a nonnaturally occurring component.

The fermenting organism described herein may be in any viable form, including crumbled, dry, including active dry and instant, compressed, cream (liquid) form etc. In one embodiment, the fermenting organism (e.g., a Saccharomyces cerevisiae yeast strain) is dry yeast, such as active dry yeast or instant yeast. In one embodiment, the fermenting organism (e.g., a Saccharomyces cerevisiae yeast strain) is crumbled yeast. In one embodiment, the fermenting organism (e.g., a Saccharomyces cerevisiae yeast strain) is compressed yeast. In one embodiment, the fermenting organism (e.g., a Saccharomyces cerevisiae yeast strain) is cream yeast.

In one embodiment is a composition comprising a fermenting organism described herein (e.g., a Saccharomyces cerevisiae yeast strain), and one or more of the component selected from the group consisting of: surfactants, emulsifiers, gums, swelling agent, and antioxidants and other processing aids.

The compositions described herein may comprise a fermenting organism described herein (e.g., a Saccharomyces cerevisiae yeast strain) and any suitable surfactants. In one embodiment, the surfactant(s) is/are an anionic surfactant, cationic surfactant, and/or nonionic surfactant.

The compositions described herein may comprise a fermenting organism described herein (e.g., a Saccharomyces cerevisiae yeast strain) and any suitable emulsifier. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

### Fermentation

The fermentation conditions are determined based on, e.g., the kind of plant material, the available fermentable sugars, the fermenting organism(s) and/or the desired fermentation product. One skilled in the art can easily determine suitable fermentation conditions. The fermentation may be carried out at conventionally used conditions. Preferred fermentation processes are anaerobic processes.

For example, fermentations may be carried out at temperatures as high as 75° C., e.g., between 40-70° C., such as between 50-60° C. However, bacteria with a significantly lower temperature optimum down to around room temperature (around 20° C.) are also known. Examples of suitable fermenting organisms can be found in the “Fermenting Organisms” section above.

For ethanol production using yeast, the fermentation may go on for 24 to 96 hours, in particular for 35 to 60 hours. In an embodiment the fermentation is carried out at a temperature between 20 to 40° C., preferably 26 to 34° C., in particular around 32° C. In an embodiment the pH is from pH 3 to 6, preferably around pH 4 to 5.

### Recovery of Fermentation Products

Subsequent to fermentation or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art. Typically, the fermentation product, e.g., ethanol, with a purity of up to, e.g., about 96 vol. percent ethanol is obtained.

Thus, in one embodiment, the method of the invention further comprises distillation to obtain the fermentation product, e.g., ethanol. The fermentation and the distillation may be carried out simultaneously and/or separately/sequentially; optionally followed by one or more process steps for further refinement of the fermentation product.

Following the completion of the distillation process, the material remaining is considered the whole stillage. As used herein, the term “whole stillage” includes the material that remains at the end of the distillation process after recovery of the fermentation product, e.g., ethanol. The fermentation product can optionally be recovered by any method known in the art.

Separating (Dewatering) Whole Stillage into Thin Stillage and Wet Cake

In one embodiment, the whole stillage is separated or partitioned into a solid and liquid phase by one or more methods for separating the thin stillage from the wet cake. Separating whole stillage into thin stillage and wet cake in order to remove a significant portion of the liquid/water, may be done using any suitable separation technique, including centrifugation, pressing and filtration. In a preferred embodiment, the separation/dewatering is carried out by centrifugation. Preferred centrifuges in industry are decanter type centrifuges, preferably high speed decanter type centrifuges. An example of a suitable centrifuge is the NX 400 steep cone series from Alfa Laval which is a high-performance decanter. In another preferred embodiment, the separation is carried out using other conventional separation equipment such as a plate/frame filter presses, belt filter presses, screw presses, gravity thickeners and deckers, or similar equipment.

### Processing of Thin Stillage

Thin stillage is the term used for the supernatant of the centrifugation of the whole stillage. Typically, the thin stillage contains 4-6 percent dry solids (DS) (mainly proteins, soluble fiber, fine fibers, and cell wall components) and has a temperature of about 60-90 degrees centigrade. The thin stillage stream may be condensed by evaporation to provide two process streams including: (i) an evaporator condensate stream comprising condensed water removed from the thin stillage during evaporation, and (ii) a syrup stream, comprising a more concentrated stream of the non-volatile dissolved and non-dissolved solids, such as non-fermentable sugars and oil, remaining present from the thin stillage as the result of removing the evaporated water. Optionally, oil can be removed from the thin stillage or can be removed as an intermediate step to the evaporation process, which is typically carried out using a series of several evaporation stages. Syrup and/or de-oiled syrup may be introduced into a dryer together with the wet grains (from the whole stillage separation step) to provide a product referred to as distillers dried grain with solubles, which also can be used as animal feed.

In an embodiment, syrup and/or de-oiled syrup is sprayed into one or more dryers to combine the syrup and/or de-oiled syrup with the whole stillage to produce distillers dried grain with solubles.

Between 5-90 vol-%, such as between 10-80%, such as between 15-70%, such as between 20-60% of thin stillage (e.g., optionally hydrolyzed) may be recycled (as backset) to step (a). The recycled thin stillage (i.e., backset) may constitute from about 1-70 vol.-%, preferably 15-60% vol.-%, especially from about 30 to 50 vol.-% of the slurry formed in step (a).

In an embodiment, the process further comprises recycling at least a portion of the thin stillage stream treated with a LPMO of the invention to the slurry, optionally after oil has been extracted from the thin stillage stream.

Drying of Wet Cake and Producing Distillers Dried Grains and Distillers Dried Grains with Solubles

After the wet cake, containing about 25-40 wt-%, preferably 30-38 wt-% dry solids, has been separated from the thin stillage (e.g., dewatered) it may be dried in a drum dryer, spray dryer, ring drier, fluid bed drier or the like in order to produce “Distillers Dried Grains” (DDG). DDG is a valuable feed ingredient for animals, such as livestock, poultry and fish. It is preferred to provide DDG with a content of less than about 10-12 wt.-% moisture to avoid mold and microbial breakdown and increase the shelf life. Further, high moisture content also makes it more expensive to transport DDG. The wet cake is preferably dried under conditions that do not denature proteins in the wet cake. The wet cake may be blended with syrup separated from the thin stillage and dried into DDG with Solubles (DDGS). Partially dried intermediate products, such as are sometimes referred to as modified wet distillers grains, may be produced by partially drying wet cake, optionally with the addition of syrup before, during or after the drying process.

Alpha-Amylase Present and/or Added During Liquefaction

According to the invention an alpha-amylase is present and/or added in liquefaction optionally together with a hemicellulase, an endoglucanase, a protease, a carbohydrate-source generating enzyme, such as a glucoamylase, a phospholipase, a phytase, and/or pullulanase.

The alpha-amylase added during liquefaction step i) may be any alpha-amylase. Preferred are bacterial alpha-amylases, such as especially Bacillus alpha-amylases, such as Bacillus stearothermophilus alpha-amylases, which are stable at temperature used during liquefaction.

### Bacterial Alpha-Amylase

The term “bacterial alpha-amylase” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment the Bacillus alpha-amylase is derived from a strain of Bacillus amyloliquefaciens, Bacillus licheniformis, Bacillus stearothermophilus, Bacillus sp. TS-23, or Bacillus subtilis, but may also be derived from other Bacillus sp.

Specific examples of bacterial alpha-amylases include the Bacillus stearothermophilus alpha-amylase of SEQ ID NO: 3 in WO 99/19467 or SEQ ID NO: 10 herein, the Bacillus amyloliquefaciens alpha-amylase of SEQ ID NO: 5 in WO 99/19467, and the Bacillus licheniformis alpha-amylase of SEQ ID NO: 4 in WO 99/19467 and the Bacillus sp. TS-23 alpha-amylase disclosed as SEQ ID NO: 1 in WO 2009/061380 (all sequences are hereby incorporated by reference).

In an embodiment the bacterial alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467 and SEQ ID NO: 1 in WO 2009/061380.

In an embodiment the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO 99/19467 or SEQ ID NO: 10 herein.

In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases, or variant thereof, may be naturally truncated during recombinant production. For instance, the mature Bacillus stearothermophilus alpha-amylase may be truncated at the C-terminal so it is around 491 amino acids long (compared to SEQ ID NO: 3 in WO 99/19467 or SEQ ID NO: 10 herein), such as from 480-495 amino acids long.

The Bacillus alpha-amylase may also be a variant and/or hybrid. Examples of such a variant can be found in any of WO 96/23873, WO 96/23874, WO 97/41213, WO 99/19467, WO 00/60059, WO 02/10355 and WO2009/061380 (all documents are hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at any of positions R179, G180, I181 and/or G182, preferably the double deletion disclosed in WO 96/23873—see, e.g., page 20, lines 1-10 (hereby incorporated by reference), preferably corresponding to deletion of positions I181 and G182 compared to the amino acid sequence of Bacillus stearothermophilus alpha-amylase set forth in SEQ ID NO: 3 disclosed in WO 99/19467 or SEQ ID NO: 10 herein or the deletion of amino acids R179 and G180 using SEQ ID NO: 3 in WO 99/19467 or SEQ ID NO: 10 herein. Even more preferred are Bacillus alpha-amylases, especially Bacillus stearothermophilus (BSG) alpha-amylases, which have at one or two amino acid deletions corresponding to positions R179, G180, I181 and G182, preferably which have a double deletion corresponding to R179 and G180, or preferably a deletion of positions 181 and 182 (denoted I181*+G182*), and optionally further comprises a N193F substitution (denoted I181*+G182*+N193F) compared to the wild-type BSG alpha-amylase amino acid sequence set forth in SEQ ID NO: 3 disclosed in WO 99/19467 or SEQ ID NO: 10 herein. The bacterial alpha-amylase may also have a substitution in a position corresponding to S239 in the Bacillus licheniformis alpha-amylase shown in SEQ ID NO: 4 in WO 99/19467, or a S242 variant in the Bacillus stearothermophilus alpha-amylase of SEQ ID NO: 3 in WO 99/19467 or SEQ ID NO: 10 herein.

In an embodiment the variant is a S242A, E or Q variant, preferably a S242Q or A variant, of the Bacillus stearothermophilus alpha-amylase (using SEQ ID NO: 10 herein for numbering).

In an embodiment the variant is a position E188 variant, preferably E188P variant of the Bacillus stearothermophilus alpha-amylase (using SEQ ID NO: 10 herein for numbering).

Other contemplated variant are Bacillus sp. TS-23 variant disclosed in WO2009/061380, especially variants defined in claim 1 of WO2009/061380 (hereby incorporated by reference).

### Bacterial Hybrid Alpha-Amylases

The bacterial alpha-amylase may also be a hybrid bacterial alpha-amylase, e.g., an alpha-amylase comprising 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467). In a preferred embodiment this hybrid has one or more, especially all, of the following substitutions:

G48A+T49I+G107A+H156Y+A181T+N190F+I201F+A209V+Q264S (using the Bacillus licheniformis numbering in SEQ ID NO: 4 of WO 99/19467). Also preferred are variants having one or more of the following mutations (or corresponding mutations in other Bacillus alpha-amylases): H154Y, A181T, N190F, A209V and Q264S and/or the deletion of two residues between positions 176 and 179, preferably the deletion of E178 and G179 (using SEQ ID NO: 5 of WO 99/19467 for position numbering).

In an embodiment the bacterial alpha-amylase is the mature part of the chimeric alpha-amylase disclosed in Richardson et al., 2002, The Journal of Biological Chemistry 277(29):. 267501-26507, referred to as BD5088 or a variant thereof. This alpha-amylase is the same as the one shown in SEQ ID NO: 2 in WO 2007134207. The mature enzyme sequence starts after the initial “Met” amino acid in position 1.

### Thermostable Alpha-Amylase

According to the invention the alpha-amylase is used in combination with a hemicellulase, preferably xylanase, having a Melting Point (DSC) above 80° C. Optionally an endoglucanase having a Melting Point (DSC) above 70° C., such as above 75° C., in particular above 80° C. may be included. The thermostable alpha-amylase, such as a bacterial an alpha-amylase, is preferably derived from Bacillus stearothermophilus or Bacillus sp. TS-23.

In an embodiment the alpha-amylase has a T½ (min) at pH 4.5, 85° C., 0.12 mM CaCl₂ of at least 10.

In an embodiment the alpha-amylase has a T½ (min) at pH 4.5, 85° C., 0.12 mM CaCl₂, of at least 15.

In an embodiment the alpha-amylase has a T½ (min) at pH 4.5, 85° C., 0.12 mM CaCl₂, of at least 20.

In an embodiment the alpha-amylase has a T½ (min) at pH 4.5, 85° C., 0.12 mM CaCl₂, of at least 25.

In an embodiment the alpha-amylase has a T½ (min) at pH 4.5, 85° C., 0.12 mM CaCl₂, of at least 30.

In an embodiment the alpha-amylase has a T½ (min) at pH 4.5, 85° C., 0.12 mM CaCl₂, of at least 40.

In an embodiment the alpha-amylase has a T½ (min) at pH 4.5, 85° C., 0.12 mM CaCl₂, of at least 50.

In an embodiment the alpha-amylase has a T½ (min) at pH 4.5, 85° C., 0.12 mM CaCl₂, of at least 60.

In an embodiment the alpha-amylase has a T½ (min) at pH 4.5, 85° C., 0.12 mM CaCl₂, between 10-70.

In an embodiment the alpha-amylase has a T½ (min) at pH 4.5, 85° C., 0.12 mM CaCl₂, between 15-70.

In an embodiment the alpha-amylase has a T½ (min) at pH 4.5, 85° C., 0.12 mM CaCl₂, between 20-70.

In an embodiment the alpha-amylase has a T½ (min) at pH 4.5, 85° C., 0.12 mM CaCl₂, between 25-70.

In an embodiment the alpha-amylase has a T½ (min) at pH 4.5, 85° C., 0.12 mM CaCl₂, between 30-70.

In an embodiment the alpha-amylase has a T½ (min) at pH 4.5, 85° C., 0.12 mM CaCl₂, between 40-70.

In an embodiment the alpha-amylase has a T½ (min) at pH 4.5, 85° C., 0.12 mM CaCl₂, between 50-70.

In an embodiment the alpha-amylase has a T½ (min) at pH 4.5, 85° C., 0.12 mM CaCl₂, between 60-70.

In an embodiment the alpha-amylase is a bacterial alpha-amylase, preferably derived from the genus Bacillus, especially a strain of Bacillus stearothermophilus, in particular the Bacillus stearothermophilus as disclosed in WO 99/19467 as SEQ ID NO: 3 or SEQ ID NO: 10 herein with one or two amino acids deleted at positions R179, G180, I181 and/or G182, in particular with R179 and G180 deleted, or with I181 and G182 deleted, with mutations in below list of mutations. In preferred embodiments the Bacillus stearothermophilus alpha-amylases have double deletion I181+G182, and optional substitution N193F, optionally further comprising mutations selected from below list:

V59A + Q89R + G112D + E129V + K177L + R179E + K220P + N224L + Q254S;
V59A + Q89R + E129V + K177L + R179E + H208Y + K220P + N224L + Q254S;
V59A + Q89R + E129V + K177L + R179E + K220P + N224L + Q254S + D269E + D281N;
V59A + Q89R + E129V + K177L + R179E + K220P + N224L + Q254S + I270L;
V59A + Q89R + E129V + K177L + R179E + K220P + N224L + Q254S + H274K;
V59A + Q89R + E129V + K177L + R179E + K220P + N224L + Q254S + Y276F;
V59A + E129V + R157Y + K177L + R179E + K220P + N224L + S242Q + Q254S;
V59A + E129V + K177L + R179E + H208Y + K220P + N224L + S242Q + Q254S;
V59A + E129V + K177L + R179E + K220P + N224L + S242Q + Q254S;
V59A + E129V + K177L + R179E + K220P + N224L + S242Q + Q254S + H274K;
V59A + E129V + K177L + R179E + K220P + N224L + S242Q + Q254S + Y276F;
V59A + E129V + K177L + R179E + K220P + N224L + S242Q + Q254S + D281N;
V59A + E129V + K177L + R179E + K220P + N224L + S242Q + Q254S + M284T;
V59A + E129V + K177L + R179E + K220P + N224L + S242Q + Q254S + G416V;
V59A + E129V + K177L + R179E + K220P + N224L + Q254S;
V59A + E129V + K177L + R179E + K220P + N224L + Q254S + M284T;
A91L + M96I + E129V + K177L + R179E + K220P + N224L + S242Q + Q254S;
E129V + K177L + R179E;
E129V + K177L + R179E + K220P + N224L + S242Q + Q254S;
E129V + K177L + R179E + K220P + N224L + S242Q + Q254S + Y276F + L427M;
E129V + K177L + R179E + K220P + N224L + S242Q + Q254S + M284T;
E129V + K177L + R179E + K220P + N224L + S242Q + Q254S + N376* + l377*;
E129V + K177L + R179E + K220P + N224L + Q254S;
E129V + K177L + R179E + K220P + N224L + Q254S + M284T;
E129V + K177L + R179E + S242Q;
E129V + K177L + R179V + K220P + N224L + S242Q + Q254S;
K220P + N224L + S242Q + Q254S;
M284V;
V59A + Q89R + E129V + K177L + R179E + Q254S + M284V.

In an embodiment the alpha-amylase is selected from the group of Bacillus stearomthermphilus alpha-amylase variants:
I181*+G182*;
I181*+G182*+N193F;
   preferably
I181*+G182*+E129V+K177L+R179E;
I181*+G182*+N193F+E129V+K177L+R179E;
I181*+G182*+N193F+V59A+Q89R+E129V+K177L+R179E+H208Y+K220P+N224L+Q254S;
I181*+G182*+N193F+V59A+Q89R+E129V+K177L+R179E+Q254S+M284V; and
I181*+G182*+N193F+E129V+K177L+R179E+K220P+N224L+S242Q+Q254S (using SEQ ID NO: 10 herein for numbering).

In an embodiment the bacterial alpha-amylase, such as Bacillus alpha-amylase, such as as Bacillus stearomthermphilus alpha-amylase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 10 herein.

In an embodiment the bacterial alpha-amylase variant, such as Bacillus alpha-amylase variant, such as Bacillus stearomthermphilus alpha-amylase variant has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 10 herein.

It should be understood that when referring to Bacillus stearothermophilus alpha-amylase and variants thereof they are normally produced naturally in truncated form. In particular, the truncation may be so that the Bacillus stearothermophilus alpha-amylase shown in SEQ ID NO: 3 in WO 99/19467 or SEQ ID NO: 10 herein, or variants thereof, are truncated in the C-terminal and are typically around 491 amino acids long, such as from 480-495 amino acids long.

Thermostable Hemicellulase Present and/or Added During Liquefaction

According to the invention an optional hemicellulase, preferably xylanase, having a Melting Point (DSC) above 80° C. is present and/or added to liquefaction step i) in combination with an alpha-amylase, such as a bacterial alpha-amylase (described above).

The thermostability of a hemicellulase, preferably xylanase may be determined as described in the “Materials & Methods”-section under “Determination of T_{d} by Differential Scanning calorimetry for Endoglucanases and Hemicellulases”.

In an embodiment the hemicellulase, in particular xylanase, especially GH10 or GH11 xylanase has a Melting Point (DSC) above 82° C., such as above 84° C., such as above 86° C., such as above 88° C., such as above 88° C., such as above 90° C., such as above 92° C., such as above 94° C., such as above 96° C., such as above 98° C., such as above 100° C., such as between 80° C. and 110° C., such as between 82° C. and 110° C., such as between 84° C. and 110° C.

In a preferred embodiment the hemicellulase, in particular xylanase, especially GH10 xylanase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 11 herein, preferably derived from a strain of the genus Dictyoglomus, such as a strain of Dictyogllomus thermophilum.

In a preferred embodiment the hemicellulase, in particular xylanase, especially GH11 xylanase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 12 herein, preferably derived from a strain of the genus Dictyoglomus, such as a strain of Dictyogllomus thermophilum.

In a preferred embodiment the hemicellulase, in particular xylanase, especially GH10 xylanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 13 herein, preferably derived from a strain of the genus Rasamsonia, such as a strain of Rasomsonia byssochlamydoides.

In a preferred embodiment the hemicellulase, in particular xylanase, especially GH10 xylanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 14 herein, preferably derived from a strain of the genus Talaromyces, such as a strain of Talaromyces leycettanus.

In a preferred embodiment the hemicellulase, in particular xylanase, especially GH10 xylanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 15 herein, preferably derived from a strain of the genus Aspergillus, such as a strain of Aspergillus fumigatus.

Thermostable Endoglucanase Present and/or Added During Liquefaction

According to the invention an optional endoglucanase (“E”) having a Melting Point (DSC) above 70° C., such as between 70° C. and 95° C. may be present and/or added in liquefaction step i) in combination with an alpha-amylase, such as a thermostable bacterial alpha-amylase and an optional hemicellulase, preferably xylanase, having a Melting Point (DSC) above 80° C.

The thermostability of an endoglucanase may be determined as described in the “Materials & Methods”-section of WO 2017/112540 (incorporated herein by reference in its entirety) under the heading “Determination of T_{d} by Differential Scanning calorimetry for Endoglucanases and Hemicellulases”.

In an embodiment the endoglucanase has a Melting Point (DSC) above 72° C., such as above 74° C., such as above 76° C., such as above 78° C., such as above 80° C., such as above 82° C., such as above 84° C., such as above 86° C., such as above 88° C., such as between 70° C. and 95° C., such as between 76° C. and 94° C., such as between 78° C. and 93° C., such as between 80° C. and 92° C., such as between 82° C. and 91° C., such as between 84° C. and 90° C.

In a preferred embodiment the endoglucanase used in a process of the invention or comprised in a composition of the invention is a Glycoside Hydrolase Family 5 endoglucanase or GH5 endoglucanase (see the CAZy database on the “www.cazy.org” webpage.

In an embodiment the GH5 endoglucanase is from family EG II, such as the Talaromyces leycettanus endoglucanase shown in SEQ ID NO: 16 herein; Penicillium capsulatum endoglucanase shown in SEQ ID NO: 17 herein, and Trichophaea saccata endoglucanase shown in SEQ ID NO: 18 herein.

In an embodiment the endoglucanase is a family GH45 endoglucanase. In an embodiment the GH45 endoglucanase is from family EG V, such as the Sordaria fimicola shown in SEQ ID NO: 19 herein or the Thielavia terrestris endoglucanase shown in SEQ ID NO: 20 herein.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 16 herein. In an embodiment the endoglucanase is derived from a strain of the genus Talaromyces, such as a strain of Talaromyces leycettanus.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 17 herein, preferably derived from a strain of the genus Penicillium, such as a strain of Penicillium capsulatum.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 18 herein, preferably derived from a strain of the genus Trichophaea, such as a strain of Trichophaea saccata.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 19 herein, preferably derived from a strain of the genus Sordaria, such as a strain of Sordaria fimicola.

In an embodiment the endoglucanase has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 20 herein, preferably derived from a strain of the genus Thielavia, such as a strain of Thielavia terrestris.

In an embodiment the endoglucanase is added in liquefaction step i) at a dose from 1-10,000 μg EP (Enzymes Protein)/g DS), such as 10-1,000 μg EP/g DS.

Carbohydrate-Source Generating Enzyme Present and/or Added During Liquefaction

According to the invention an optional carbohydrate-source generating enzyme, in particular a glucoamylase, preferably a thermostable glucoamylase, may be present and/or added in liquefaction together with an alpha-amylase and optional hemicellulase, preferably xylanase, having a Melting Point (DSC) above 80° C., and an optional endoglucanase having a Melting Point (DSC) above 70° C., and an optional a pullulanase and/or optional phytase.

The term “carbohydrate-source generating enzyme” includes any enzymes generating fermentable sugars. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrates may be converted directly or indirectly to the desired fermentation product, preferably ethanol. According to the invention a mixture of carbohydrate-source generating enzymes may be used. Specific examples include glucoamylase (being glucose generators), beta-amylase and maltogenic amylase (being maltose generators).

In a preferred embodiment the carbohydrate-source generating enzyme is thermostable. The carbohydrate-source generating enzyme, in particular thermostable glucoamylase, may be added together with or separately from the alpha-amylase and the thermostable protease.

In a specific and preferred embodiment the carbohydrate-source generating enzyme is a thermostable glucoamylase, preferably of fungal origin, preferably a filamentous fungi, such as from a strain of the genus Penicillium, especially a strain of Penicillium oxalicum, in particular the Penicillium oxalicum glucoamylase disclosed as SEQ ID NO: 2 in WO 2011/127802 (which is hereby incorporated by reference) and shown in SEQ ID NO: 21 herein.

In an embodiment the thermostable glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802 or SEQ ID NOs: 21 herein.

In an embodiment the carbohydrate-source generating enzyme, in particular thermostable glucoamylase, is the Penicillium oxalicum glucoamylase shown in SEQ ID NO: 21 herein.

In a preferred embodiment the carbohydrate-source generating enzyme is a variant of the Penicillium oxalicum glucoamylase disclosed as SEQ ID NO: 2 in WO 2011/127802 and shown in SEQ ID NO: 21 herein, having a K79V substitution (referred to as “PE001”) (using the mature sequence shown in SEQ ID NO: 14 for numbering). The K79V glucoamylase variant has reduced sensitivity to protease degradation relative to the parent as disclosed in WO 2013/036526 (which is hereby incorporated by reference).

Contemplated Penicillium oxalicum glucoamylase variants are disclosed in WO 2013/053801 (which is hereby incorporated by reference).

In an embodiment these variants have reduced sensitivity to protease degradation.

In an embodiment these variant have improved thermostability compared to the parent.

More specifically, in an embodiment the glucoamylase has a K79V substitution (using SEQ ID NO: 21 herein for numbering), corresponding to the PE001 variant, and further comprises at least one of the following substitutions or combination of substitutions:

T65A; Q327F; E501V; Y504T; Y504*; T65A+Q327F; T65A+E501V; T65A+Y504T; T65A+Y504*; Q327F+E501V; Q327F+Y504T; Q327F+Y504*; E501V+Y504T; E501V+Y504*; T65A+Q327F+E501V; T65A+Q327F+Y504T; T65A+E501V+Y504T; Q327F+E501V+Y504T; T65A+Q327F+Y504*; T65A+E501V+Y504*; Q327F+E501V+Y504*; T65A+Q327F+E501V+Y504T; T65A+Q327F+E501V+Y504*; E501V+Y504T; T65A+K161S; T65A+Q405T; T65A+Q327W; T65A+Q327F; T65A+Q327Y; P11F+T65A+Q327F;
R1K+D3W+K5Q+G7V+N8S+T10K+P11S+T65A+Q327F; P2N+P4S+P11F+T65A+Q327F; P11F+D26C+K33C+T65A+Q327F; P2N+P4S+P11F+T65A+Q327W+E501V+Y504T; R1E+D3N+P4G+G6R+G7A+N8A+T10D+P11D+T65A+Q327F; P11F+T65A+Q327W; P2N+P4S+P11F+T65A+Q327F+E501V+Y504T; P11F+T65A+Q327W+E501V+Y504T; T65A+Q327F+E501V+Y504T; T65A+S105P+Q327W; T65A+S105P+Q327F; T65A+Q327W+S364P; T65A+Q327F+S364P; T65A+S103N+Q327F; P2N+P4S+P11F+K34Y+T65A+Q327F; P2N+P4S+P11F+T65A+Q327F+D445N+V447S; P2N+P4S+P11F+T65A+I172V+Q327F; P2N+P4S+P11F+T65A+Q327F+N502*; P2N+P4S+P11F+T65A+Q327F+N502T+P563S+K571E; P2N+P4S+P11F+R31S+K33V+T65A+Q327F+N564D+K571S; P2N+P4S+P11F+T65A+Q327F+S377T; P2N+P4S+P11F+T65A+V325T+Q327W; P2N+P4S+P11F+T65A+Q327F+D445N+V447S+E501V+Y504T; P2N+P4S+P11F+T65A+I172V+Q327F+E501V+Y504T; P2N+P4S+P11F+T65A+Q327F+S377T+E501V+Y504T; P2N+P4S+P11F+D26N+K34Y+T65A+Q327F; P2N+P4S+P11F+T65A+Q327F+I375A+E501V+Y504T; P2N+P4S+P11F+T65A+K218A+K221D+Q327F+E501V+Y504T; P2N+P4S+P11F+T65A+S103N+Q327F+E501V+Y504T; P2N+P4S+T10D+T65A+Q327F+E501V+Y504T; P2N+P4S+F12Y+T65A+Q327F+E501V+Y504T; K5A+P11F+T65A+Q327F+E501V+Y504T; P2N+P4S+T10E+E18N+T65A+Q327F+E501V+Y504T; P2N+T10E+E18N+T65A+Q327F+E501V+Y504T; P2N+P4S+P11F+T65A+Q327F+E501V+Y504T+T568N; P2N+P4S+P11F+T65A+Q327F+E501V+Y504T+K524T+G526A; P2N+P4S+P11F+K34Y+T65A+Q327F+D445N+V447S+E501V+Y504T; P2N+P4S+P11F+R31S+K33V+T65A+Q327F+D445N+V447S+E501V+Y504T; P2N+P4S+P11F+D26N+K34Y+T65A+Q327F+E501V+Y504T; P2N+P4S+P11F+T65A+F80*+Q327F+E501V+Y504T; P2N+P4S+P11F+T65A+K112S+Q327F+E501V+Y504T; P2N+P4S+P11F+T65A+Q327F+E501V+Y504T+T516P+K524T+G526A; P2N+P4S+P11F+T65A+Q327F+E501V+N502T+Y504*; P2N+P4S+P11F+T65A+Q327F+E501V+Y504T; P2N+P4S+P11F+T65A+S103N+Q327F+E501V+Y504T; K5A+P11F+T65A+Q327F+E501V+Y504T; P2N+P4S+P11F+T65A+Q327F+E501V+Y504T+T516P+K524T+G526A; P2N+P4S+P11F+T65A+V79A+Q327F+E501V+Y504T; P2N+P4S+P11F+T65A+V79G+Q327F+E501V+Y504T; P2N+P4S+P11F+T65A+V791+Q327F+E501V+Y504T; P2N+P4S+P11F+T65A+V79L+Q327F+E501V+Y504T; P2N+P4S+P11F+T65A+V79S+Q327F+E501V+Y504T; P2N+P4S+P11F+T65A+L72V+Q327F+E501V+Y504T; S255N+Q327F+E501V+Y504T; P2N+P4S+P11F+T65A+E74N+Q327F+E501V+Y504T; P2N+P4S+P11F+T65A+G220N+Q327F+E501V+Y504T; P2N+P4S+P11F+T65A+Y245N+Q327F+E501V+Y504T; P2N+P4S+P11F+T65A+Q253N+Q327F+E501V+Y504T; P2N+P4S+P11F+T65A+D279N+Q327F+E501V+Y504T; P2N+P4S+P11F+T65A+Q327F+S359N+E501V+Y504T; P2N+P4S+P11F+T65A+Q327F+D370N+E501V+Y504T; P2N+P4S+P11F+T65A+Q327F+V460S+E501V+Y504T; P2N+P4S+P11F+T65A+Q327F+V460T+P468T+E501V+Y504T; P2N+P4S+P11F+T65A+Q327F+T463N+E501V+Y504T; P2N+P4S+P11F+T65A+Q327F+S465N+E501V+Y504T; or P2N+P4S+P11F+T65A+Q327F+T477N+E501V+Y504T.

In a preferred embodiment the Penicillium oxalicum glucoamylase variant has a K79V substitution using SEQ ID NO: 21 herein for numbering (PE001 variant), and further comprises one of the following mutations:

### P11F+T65A+Q327F;

### P2N+P4S+P11F+T65A+Q327F;

### P11F+D26C+K330+T65A+Q327F;

### P2N+P4S+P11F+T65A+Q327W+E501V+Y504T;

### P2N+P4S+P11F+T65A+Q327F+E501V+Y504T; or

### P11F+T65A+Q327W+E501V+Y504T.

In an embodiment the glucoamylase variant, such as Penicillium oxalicum glucoamylase variant has at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature polypeptide of SEQ ID NO: 21 herein.

The carbohydrate-source generating enzyme, in particular glycoamylase, may be added in amounts from 0.1-100 micrograms EP/g DS, such as 0.5-50 micrograms EP/g DS, such as 1-25 micrograms EP/g DS, such as 2-12 micrograms EP/g DS.

Pullulanase Present and/or Added During Liquefaction

Optionally a pullulanase may be present and/or added during liquefaction step i) together with an alpha-amylase and an optional hemicellulase, preferably xylanase, having a melting point (DSC) above 80° C. As mentioned above a protease, a carbohydrate-source generating enzyme, preferably a thermostable glucoamylase, may also optionally be present and/or added during liquefaction step i).

The pullulanase may be present and/or added during liquefaction step i) and/or saccharification step ii) or simultaneous saccharification and fermentation.

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Additional pullulanases contemplated according to the present invention included the pullulanases from Pyrococcus woesei, specifically from Pyrococcus woesei DSM No. 3773 disclosed in WO 92/02614.

In an embodiment the pullulanase is a family GH57 pullulanase. In an embodiment the pullulanase includes an X47 domain as disclosed in WO 2011/087836 (which are hereby incorporated by reference). More specifically the pullulanase may be derived from a strain of the genus Thermococcus, including Thermococcus litoralis and Thermococcus hydrothermalis, such as the Thermococcus hydrothermalis pullulanase shown WO 2011/087836 truncated at the X4 site right after the X47 domain. The pullulanase may also be a hybrid of the Thermococcus litoralis and Thermococcus hydrothermalis pullulanases or a T. hydrothermalis/T. litoralis hybrid enzyme with truncation site X4 disclosed in WO 2011/087836 (which is hereby incorporated by reference).

In another embodiment the pullulanase is one comprising an X46 domain disclosed in WO 2011/076123 (Novozymes).

The pullulanase may according to the invention be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in the “Materials & Methods”-section below.

Suitable commercially available pullulanase products include PROMOZYME 400L, PROMOZYME™ D2 (Novozymes A/S, Denmark), OPTIMAX L-300 (Genencor Int., USA), and AMANO 8 (Amano, Japan).

Phytase Present and/or Added During Liquefaction

Optionally a phytase may be present and/or added in liquefaction in combination with an alpha-amylase and hemicellulase, preferably xylanase, having a melting point (DSC) above 80° C.

A phytase used according to the invention may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. The phytase to be used in the invention may have any specificity, e.g., be a 3-phytase (EC 3.1.3.8), a 6-phytase (EC 3.1.3.26) or a 5-phytase (no EC number). In an embodiment the phytase has a temperature optimum above 50° C., such as in the range from 50-90° C.

The phytase may be derived from plants or microorganisms, such as bacteria or fungi, e.g., yeast or filamentous fungi.

A plant phytase may be from wheat-bran, maize, soy bean or lily pollen. Suitable plant phytases are described in Thomlinson et al, Biochemistry, 1 (1962), 166-171; Barrientos et al, Plant. Physiol., 106 (1994), 1489-1495; WO 98/05785; WO 98/20139.

A bacterial phytase may be from genus Bacillus, Citrobacter, Hafnia, Pseudomonas, Buttiauxella or Escherichia, specifically the species Bacillus subtilis, Citrobacter braakii, Citrobacter freundii, Hafnia alvei, Buttiauxella gaviniae, Buttiauxella agrestis, Buttiauxella noackies and E. coli. Suitable bacterial phytases are described in Paver and Jagannathan, 1982, Journal of Bacteriology 151:1102-1108; Cosgrove, 1970, Australian Journal of Biological Sciences 23:1207-1220; Greiner et al, Arch. Biochem. Biophys., 303, 107-113, 1993; WO 1997/33976; WO 1997/48812, WO 1998/06856, WO 1998/028408, WO 2004/085638, WO 2006/037327, WO 2006/038062, WO 2006/063588, WO 2008/092901, WO 2008/116878, and WO 2010/034835.

A yeast phytase may be derived from genus Saccharomyces or Schwanniomyces, specifically species Saccharomyces cerevisiae or Schwanniomyces occidentalis. The former enzyme has been described as a Suitable yeast phytases are described in Nayini et al, 1984, Lebensmittel Wissenschaft and Technologie 17:24-26; Wodzinski et al, Adv. Appl. Microbiol., 42, 263-303; AU-A-24840/95;

Phytases from filamentous fungi may be derived from the fungal phylum of Ascomycota (ascomycetes) or the phylum Basidiomycota, e.g., the genus Aspergillus, Thermomyces (also called Humicola), Myceliophthora, Manascus, Penicillium, Peniophora, Agrocybe, Paxillus, or Trametes, specifically the species Aspergillus terreus, Aspergillus niger, Aspergillus niger var. awamori, Aspergillus ficuum, Aspergillus fumigatus, Aspergillus oryzae, T. lanuginosus (also known as H. lanuginosa), Myceliophthora thermophila, Peniophora lycii, Agrocybe pediades, Manascus anka, Paxillus involtus, or Trametes pubescens. Suitable fungal phytases are described in Yamada et al., 1986, Agric. Biol. Chem. 322:1275-1282; Piddington et al., 1993, Gene 133:55-62; EP 684,313; EP 0 420 358; EP 0 684 313; WO 1998/28408; WO 1998/28409; JP 7-67635; WO 1998/44125; WO 1997/38096; WO 1998/13480.

In a preferred embodiment the phytase is derived from Buttiauxella, such as Buttiauxella gaviniae, Buttiauxella agrestis, or Buttiauxella noackies, such as the ones disclosed as SEQ ID NO: 2, SEQ ID NO: 4 and SEQ ID NO: 6, respectively, in WO 2008/092901 (hereby incorporated by reference).

In a preferred embodiment the phytase is derived from Citrobacter, such as Citrobacter braakii, such as one disclosed in WO 2006/037328 (hereby incorporated by reference).

Modified phytases or phytase variants are obtainable by methods known in the art, in particular by the methods disclosed in EP 897010; EP 897985; WO 99/49022; WO 99/48330, WO 2003/066847, WO 2007/112739, WO 2009/129489, and WO 2010/034835.

Commercially available phytase containing products include BIO-FEED PHYTASE™, PHYTASE NOVO™ CT or L (all from Novozymes), LIQMAX (DuPont) or RONOZYME™ NP, RONOZYME® HiPhos, RONOZYME® P5000 (CT), NATUPHOS™ NG 5000 (from DSM).

According to the invention a carbohydrate-source generating enzyme, preferably a glucoamylase, is present and/or added during saccharification and/or fermentation.

In a preferred embodiment the carbohydrate-source generating enzyme is a glucoamylase, of fungal origin, preferably from a stain of Aspergillus, preferably A. niger, A. awamori, or A. oryzae; or a strain of Trichoderma, preferably T. reesei; or a strain of Talaromyces, preferably T. emersonii,

Carbohydrate-Source Generating Enzyme Present and/or Added During Saccharification and/or Fermentation

According to the invention a carbohydrate-source generating enzyme, in particular a glucoamylase, may be present and/or added in saccharification step (b), fermentation step (c), simultaneous saccharification and fermentation (SSF); or presaccharification before step (b) optionally together with an alpha-amylase, a cellulolytic composition, a protease, a trehalase, and any combination thereof.

The carbohydrate-source generating enzyme (e.g., glucoamylase) present and/or added during saccharification step (b); fermentation step (c); simultaneous saccharification and fermentation; or presaccharification before step (b), may be derived from any suitable source, e.g., derived from a microorganism or a plant. Preferred glucoamylases are of fungal or bacterial origin, selected from the group consisting of Aspergillus glucoamylases, in particular Aspergillus nigerG1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (US patent no. Re. 32,153), Talaromyces duponti, Talaromyces thermophilus (U.S. Pat. No. 4,587,215). In a preferred embodiment the glucoamylase used during saccharification and/or fermentation is the Talaromyces emersonii glucoamylase disclosed in WO 99/28448.

Bacterial glucoamylases contemplated include glucoamylases from the genus Clostridium, in particular C. thermoamylolyticum (EP 135,138), and C. thermohydrosulfuricum (WO 86/01831).

Contemplated fungal glucoamylases include Trametes cingulata (SEQ ID NO: 8 herein), Pachykytospora papyracea; and Leucopaxillus giganteus all disclosed in WO 2006/069289; or Peniophora rufomarginata disclosed in WO2007/124285; or a mixture thereof. Also hybrid glucoamylase are contemplated according to the invention. Examples include the hybrid glucoamylases disclosed in WO 2005/045018. Specific examples include the hybrid glucoamylase disclosed in Table 1 and 4 of Example 1 (which hybrids are hereby incorporated by reference).

In an embodiment the glucoamylase is derived from a strain of the genus Pycnoporus, in particular a strain of Pycnoporus sanguineus as described in WO 2011/066576 (SEQ ID NOs 2, 4 or 6 therein), in particular the one shown a SEQ ID NO: 7 herein (corresponding to SEQ ID NO: 4 in WO 2011/066576) or from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16 therein). In a preferred embodiment the glucoamylase is SEQ ID NO: 2 in WO 2011/068803 or SEQ ID NO: 5 herein (i.e. Gloeophyllum sepiarium glucoamylase). In a preferred embodiment the glucoamylase is SEQ ID NO: 6 herein (i.e., Gloeophyllum trabeum glucoamylase discloses as SEQ ID NO: 3 in WO2014/177546) (all references hereby incorporated by reference).

Contemplated are also glucoamylases which exhibit a high identity to any of the above mentioned glucoamylases, i.e., at least 60%, such as at least 70%, at least 75%, at least 80%, at least 85%, at least 90%, at least 95%, at least 96%, at least 97%, at least 98%, at least 99% or even 100% identity to any one of the mature parts of the enzyme sequences mentioned above, such as any of SEQ ID NOs: 4, 5, 6, 7 or 8 herein, respectively.

In an embodiment the glucoamylase used in fermentation or SSF exhibits at least 60%, such as at least 70%, at least 75%, at least 80%, at least 85%, at least 90%, at least 95%, at least 96%, at least 97%, at least 98%, at least 99% or even 100% identity to the mature part of SEQ ID NO: 4 herein.

In an embodiment the glucoamylase used in fermentation or SSF exhibits at least 60%, such as at least 70%, at least 75%, at least 80%, at least 85%, at least 90%, at least 95%, at least 96%, at least 97%, at least 98%, at least 99% or even 100% identity to the mature part of SEQ ID NO: 5 herein.

In an embodiment the glucoamylase used in fermentation or SSF exhibits at least 60%, such as at least 70%, at least 75%, at least 80%, at least 85%, at least 90%, at least 95%, at least 96%, at least 97%, at least 98%, at least 99% or even 100% identity to the mature part of SEQ ID NO: 6 herein.

In an embodiment the glucoamylase used in fermentation or SSF exhibits at least 60%, such as at least 70%, at least 75%, at least 80%, at least 85%, at least 90%, at least 95%, at least 96%, at least 97%, at least 98%, at least 99% or even 100% identity to the mature part of SEQ ID NO: 7 herein.

In an embodiment the glucoamylase used in fermentation or SSF exhibits at least 60%, such as at least 70%, at least 75%, at least 80%, at least 85%, at least 90%, at least 95%, at least 96%, at least 97%, at least 98%, at least 99% or even 100% identity to the mature part of SEQ ID NO: 8 herein.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Glucoamylases may in an embodiment be added to the saccharification and/or fermentation in an amount of 1-1,000 μg EP/g DS, preferably 10-500 μg/gDS, especially between 25-250 μg/g DS.

In an embodiment the glucoamylase is added as a blend further comprising an alpha-amylase. In a preferred embodiment the alpha-amylase is a fungal alpha-amylase, especially an acid fungal alpha-amylase. The alpha-amylase is typically a side activity.

In an embodiment the glucoamylase is a blend comprising Talaromyces emersonii glucoamylase disclosed in WO 99/28448 as SEQ ID NO: 34 or SEQ ID NO: 4 herein and Trametes cingulata glucoamylase disclosed as SEQ ID NO: 2 in WO 06/069289 and SEQ ID NO: 8 herein.

In an embodiment the glucoamylase is a blend comprising Talaromyces emersonii glucoamylase disclosed in SEQ ID NO: 4 herein, Trametes cingulata glucoamylase disclosed as SEQ ID NO: 8 herein, and Rhizomucor pusillus alpha-amylase with Aspergillus niger glucoamylase linker and SBD disclosed as V039 in Table 5 in WO 2006/069290 or SEQ ID NO: 9 herein.

In an embodiment the glucoamylase is a blend comprising Gloeophyllum sepiarium glucoamylase shown as SEQ ID NO: 5 herein and Rhizomucor pusillus with an Aspergillus niger glucoamylase linker and starch-binding domain (SBD), disclosed SEQ ID NO: 9 herein with the following substitutions: G128D+D143N.

In an embodiment the alpha-amylase may be derived from a strain of the genus Rhizomucor, preferably a strain the Rhizomucor pusillus, such as the one shown in SEQ ID NO: 3 in WO2013/006756, or the genus Meripilus, preferably a strain of Meripilus giganteus. In a preferred embodiment the alpha-amylase is derived from a Rhizomucor pusillus with an Aspergillus niger glucoamylase linker and starch-binding domain (SBD), disclosed as V039 in Table 5 in WO 2006/069290 or SEQ ID NO: 9 herein.

In an embodiment the Rhizomucor pusillus alpha-amylase or the Rhizomucor pusillus alpha-amylase with a linker and starch-binding domain (SBD), preferably Aspergillus niger glucoamylase linker and SBD, has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 3 in WO 2013/006756 for numbering or SEQ ID NO: 9 herein). In a preferred embodiment the glucoamylase blend comprises Gloeophyllum sepiarium glucoamylase (e.g., SEQ ID NO: 2 in WO 2011/068803 or SEQ ID NO: 5 herein) and Rhizomucor pusillus alpha-amylase.

In a preferred embodiment the glucoamylase blend comprises Gloeophyllum sepiarium glucoamylase shown as SEQ ID NO: 2 in WO 2011/068803 or SEQ ID NO: 5 herein and Rhizomucor pusillus with a linker and starch-binding domain (SBD), preferably Aspergillus niger glucoamylase linker and SBD, disclosed SEQ ID NO: 3 in WO 2013/006756 and SEQ ID NO: 9 herein with the following substitutions: G128D+D143N.

Commercially available compositions comprising glucoamylase include AMG 200L; AMG 300 L; SAN™′SUPER, SANT™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE™, and AMG™ E (from Novozymes A/S); OPTIDEX™ 300, GC480, GC417 (from DuPont-Danisco); AMIGASE™ and AMIGASE™ PLUS (from DSM); G-ZYME™ G900, G-ZYME™ and G990 ZR (from DuPont-Danisco).

### Beta-Amylase

A beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The carbohydrate-source generating enzyme present and/or added during saccharification and/or fermentation may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference. The maltogenic amylase may in a preferred embodiment be added in an amount of 0.05-5 mg total protein/gram DS or 0.05-5 MANU/g DS.

Cellulase or Cellulolytic Enzyme Composition Present and/or Added During Saccharification and/or Fermentation or SSF

Aspects of the invention relate to using a celluloytic composition in a process of the invention. In certain aspects, the cellulolytic composition is present and/or added during saccharification, fermentation, and/or simultaneous saccharification and fermentation. The cellulolytic composition may be present or added during saccharification, fermentation, and/or simultaneous saccharification and fermentation simultaneously or sequentially together with an alpha-amylase, a glucoamylase, a protease, a trehalase, and/or any combination thereof. The cellulolytic composition used in a process of the invention may be derived from any microorganism. As used herein, “derived from any microorganism” means that the cellulolytic composition comprises one or more enzymes that were expressed in the microorganism. For instance, a cellulolytic composition derived from a strain of Trichoderma reesei means that the cellulolytic composition comprises one or more enzymes that were expressed in Trichoderma reesei.

In an embodiment, the cellulolytic composition is derived from a strain of Aspergillus, such as a strain of Aspergillus aurantiacus, Aspergillus niger or Aspergillus oryzae.

In an embodiment, the cellulolytic composition is derived from a strain of Chrysosporium, such as a strain of Chrysosporium lucknowense.

In an embodiment, the cellulolytic composition is derived from a strain of Humicola, such as a strain of Humicola insolens.

In an embodiment, the cellulolytic composition is derived from a strain of Penicilium, such as a strain of Penicilium emersonii or Penicilium oxalicum.

In an embodiment, the cellulolytic composition is derived from a strain of Talaromyces, such as a strain of Talaromyces aurantiacus or Talaromyces emersonii.

In an embodiment, the cellulolytic composition is derived from a strain of Trichoderma, such as a strain of Trichoderma reesei.

In a preferred embodiment, the cellulolytic composition is derived from a strain of Trichoderma reesei.

The cellulolytic composition may comprise one or more of the following polypeptides, including enzymes: GH61 polypeptide having cellulolytic enhancing activity, beta-glucosidase, CBHI and CBHII, or a mixture of two, three, or four thereof.

In a preferred embodiment, the cellulolytic composition comprising a beta-glucosidase having a Relative ED50 loading value of less than 1.00, preferably less than 0.80, such as preferably less than 0.60, such as between 0.1-0.9, such as between 0.2-0.8, such as 0.30-0.70.

The cellulolytic composition may comprise some hemicellulase, such as, e.g., xylanase and/or beta-xylosidase. The hemicellulase may come from the cellulolytic composition producing organism or from other sources, e.g., the hemicellulase may be foreign to the cellulolytic composition producing organism, such as, e.g., Trichoderma reesei.

In a preferred embodiment the hemicellulase content in the cellulolytic composition constitutes less than 10 wt. % such as less than 5 wt. % of the cellulolytic composition.

In an embodiment the cellulolytic composition comprises a beta-glucosidase.

In an embodiment the cellulolytic composition comprises a GH61 polypeptide having cellulolytic enhancing activity and a beta-glucosidase.

In another embodiment the cellulolytic composition comprises a beta-glucosidase and a CBH.

In another embodiment the cellulolytic composition comprises a GH61 polypeptide having cellulolytic enhancing activity, a beta-glucosidase, and a CBHI.

In another embodiment the cellulolytic composition comprises a beta-glucosidase and a CBHI.

In another embodiment the cellulolytic composition comprises a GH61 polypeptide having cellulolytic enhancing activity, a beta-glucosidase, a CBHI, and a CBHII.

In another embodiment the cellulolytic composition comprises a beta-glucosidase, a CBHI, and a CBHII.

The cellulolytic composition may further comprise one or more enzymes selected from the group consisting of a cellulase, a GH61 polypeptide having cellulolytic enhancing activity, an esterase, an expansin, a laccase, a ligninolytic enzyme, a pectinase, a peroxidase, a protease, and a swollenin.

In an embodiment the cellulase is one or more enzymes selected from the group consisting of an endoglucanase, a cellobiohydrolase, and a beta-glucosidase.

In an embodiment the endoglucanase is an endoglucanase I.

In an embodiment the endoglucanase is an endoglucanase II.

### Beta-Glucosidase

The cellulolytic composition used according to the invention may in one embodiment comprise one or more beta-glucosidase. The beta-glucosidase may in one embodiment be one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigatus, such as such as one disclosed in WO 2005/047499 or SEQ ID NO: 22 herein or an Aspergillus fumigatus beta-glucosidase variant, such as one disclosed in WO 2012/044915 or co-pending PCT application PCT/US11/054185 (or U.S. provisional application No. 61/388,997), such as one with the following substitutions: F100D, S283G, N456E, F512Y.

In another embodiment the beta-glucosidase is derived from a strain of the genus Penicillium, such as a strain of the Penicillium brasilianum disclosed in WO 2007/019442, or a strain of the genus Trichoderma, such as a strain of Trichoderma reesei.

In an embodiment betaglucosidase is an Aspergillus fumigatus beta-glucosidase or homolog thereof selected from the group consisting of:

(i) a beta-glucosidase comprising the mature polypeptide of SEQ ID NO: 22;
(ii) a beta-glucosidase comprising an amino acid sequence having at least 70%, e.g., 75%, 80%, 85%, 90%, 91%, 92%, 93%, 94%, 95%, 96%, 97%, 98%, or 99% identity to the mature polypeptide of SEQ ID NO: 22 herein;
(iii) a beta-glucosidase encoded by a polynucleotide comprising a nucleotide sequence having at least 70%, e.g., 75%, 80%, 85%, 90%, 91%, 92%, 93%, 94%, 95%, 96%, 97%, 98%, or 99% identity to the mature polypeptide coding sequence of SEQ ID NO: 5 in WO 2013/148993; and
(iv) a beta-glucosidase encoded by a polynucleotide that hybridizes under at least high stringency conditions, e.g., very high stringency conditions, with the mature polypeptide coding sequence of SEQ ID NO: 5 in WO 2013/148993 or the full-length complement thereof.

In an embodiment the beta-glucosidase is a variant comprises a substitution at one or more (several) positions corresponding to positions 100, 283, 456, and 512 of the mature polypeptide of SEQ ID NO: 22 herein, wherein the variant has beta-glucosidase activity.

In an embodiment the parent beta-glucosidase of the variant is (a) a polypeptide comprising the mature polypeptide of SEQ ID NO: 22 herein; (b) a polypeptide having at least 80% sequence identity to the mature polypeptide of SEQ ID NO: 22 herein; (c) a polypeptide encoded by a polynucleotide that hybridizes under high or very high stringency conditions with (i) the mature polypeptide coding sequence of SEQ ID NO: 5 in WO 2013/148993, (ii) the cDNA sequence contained in the mature polypeptide coding sequence of SEQ ID NO: 5 in WO 2013/148993, or (iii) the full-length complementary strand of (i) or (ii); (d) a polypeptide encoded by a polynucleotide having at least 80% identity to the mature polypeptide coding sequence of SEQ ID NO: 5 in WO 2013/148993 or the cDNA sequence thereof; or (e) a fragment of the mature polypeptide of SEQ ID NO: 22 herein, which has beta-glucosidase activity.

In an embodiment the beta-glucosidase variant has at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100%, sequence identity to the amino acid sequence of the parent beta-glucosidase.

In an embodiment the variant has at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the mature polypeptide of SEQ ID NO: 22 herein.

In an embodiment the beta-glucosidase is from a strain of Aspergillus, such as a strain of Aspergillus fumigatus, such as Aspergillus fumigatus beta-glucosidase (SEQ ID NO: 22 herein), which comprises one or more substitutions selected from the group consisting of L89M, G91L, F100D, I140V, I186V, S283G, N456E, and F512Y; such as a variant thereof with the following substitutions:
F100D+S283G+N456E+F512Y;
L89M+G91L+I 186V+I140V;
I186V+L89M+G91L+I140V+F100D+S283G+N456E+F512Y.

In an embodiment the number of substitutions is between 1 and 4, such as 1, 2, 3, or 4 substitutions.

In an embodiment the variant comprises a substitution at a position corresponding to position 100, a substitution at a position corresponding to position 283, a substitution at a position corresponding to position 456, and/or a substitution at a position corresponding to position 512.

In a preferred embodiment the beta-glucosidase variant comprises the following substitutions: Phe100Asp, Ser283Gly, Asn456Glu, Phe512Tyr in SEQ ID NO: 22 herein.

In a preferred embodiment the beta-glucosidase has a Relative ED50 loading value of less than 1.00, preferably less than 0.80, such as preferably less than 0.60, such as between 0.1-0.9, such as between 0.2-0.8, such as 0.30-0.70.

### GH61 Polypeptide Having Cellulolytic Enhancing Activity

The cellulolytic composition used according to the invention may in one embodiment comprise one or more GH61 polypeptide having cellulolytic enhancing activity. In one embodiment the enzyme composition comprises a GH61 polypeptide having cellulolytic enhancing activity, such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigatus, such as the one described in WO 2010/138754 as SEQ ID NO: 2; or one derived from a strain derived from Penicillium, such as a strain of Penicillium emersonii, such as the one disclosed in WO 2011/041397 or SEQ ID NO: 23 herein.

In an embodiment the Penicillium sp. GH61 polypeptide having cellulolytic enhancing activity or homolog thereof is selected from the group consisting of:

(i) a GH61 polypeptide having cellulolytic enhancing activity comprising the mature polypeptide of SEQ ID NO: 23 herein;
(ii) a GH61 polypeptide having cellulolytic enhancing activity comprising an amino acid sequence having at least 70%, e.g., 75%, 80%, 85%, 90%, 91%, 92%, 93%, 94%, 95%, 96%, 97%, 98%, or 99% identity to the mature polypeptide of SEQ ID NO: 23 herein;
(iii) a GH61 polypeptide having cellulolytic enhancing activity encoded by a polynucleotide comprising a nucleotide sequence having at least 70%, e.g., 75%, 80%, 85%, 90%, 91%, 92%, 93%, 94%, 95%, 96%, 97%, 98%, or 99% identity to the mature polypeptide coding sequence of SEQ ID NO: 7 in WO 2013/148993; and
(iv) a GH61 polypeptide having cellulolytic enhancing activity encoded by a polynucleotide that hybridizes under at least high stringency conditions, e.g., very high stringency conditions, with the mature polypeptide coding sequence of SEQ ID NO: 7 in WO 2013/148993 or the full-length complement thereof.

### Cellobiohydrolase I

The cellulolytic composition used according to the invention may in one embodiment may comprise one or more CBH I (cellobiohydrolase I). In one embodiment the cellulolytic composition comprises a cellobiohydrolase I (CBHI), such as one derived from a strain of the genus Aspergillus, such as a strain of Aspergillus fumigatus, such as the Cel7A CBHI disclosed in SEQ ID NO: 6 in WO 2011/057140 or SEQ ID NO: 24 herein, or a strain of the genus Trichoderma, such as a strain of Trichoderma reesei.

In an embodiment the Aspergillus fumigatus cellobiohydrolase I or homolog thereof is selected from the group consisting of:

(i) a cellobiohydrolase I comprising the mature polypeptide of SEQ ID NO: 24 herein;
(ii) a cellobiohydrolase I comprising an amino acid sequence having at least 70%, e.g., 75%, 80%, 85%, 90%, 91%, 92%, 93%, 94%, 95%, 96%, 97%, 98%, or 99% identity to the mature polypeptide of SEQ ID NO: 24 herein;
(iii) a cellobiohydrolase I encoded by a polynucleotide comprising a nucleotide sequence having at least 70%, e.g., 75%, 80%, 85%, 90%, 91%, 92%, 93%, 94%, 95%, 96%, 97%, 98%, or 99% identity to the mature polypeptide coding sequence of SEQ ID NO: 1 in WO 2013/148993; and
(iv) a cellobiohydrolase I encoded by a polynucleotide that hybridizes under at least high stringency conditions, e.g., very high stringency conditions, with the mature polypeptide coding sequence of SEQ ID NO: 1 in WO 2013/148993 or the full-length complement thereof.

### Cellobiohydrolase II

The cellulolytic composition used according to the invention may in one embodiment comprise one or more CBH II (cellobiohydrolase II). In one embodiment the cellobiohydrolase II (CBHII), such as one derived from a strain of the genus Aspergillus, such as a strain of Aspergillus fumigatus, such as the one in SEQ ID NO: 25 herein or a strain of the genus Trichoderma, such as Trichoderma reesei, or a strain of the genus Thielavia, such as a strain of Thielavia terrestris, such as cellobiohydrolase II CEL6A from Thielavia terrestris.

In an embodiment the Aspergillus fumigatus cellobiohydrolase II or homolog thereof is selected from the group consisting of:

(i) a cellobiohydrolase II comprising the mature polypeptide of SEQ ID NO: 25 herein;
(ii) a cellobiohydrolase II comprising an amino acid sequence having at least 70%, e.g., 75%, 80%, 85%, 90%, 91%, 92%, 93%, 94%, 95%, 96%, 97%, 98%, or 99% identity to the mature polypeptide of SEQ ID NO: 25 herein;
(iii) a cellobiohydrolase II encoded by a polynucleotide comprising a nucleotide sequence having at least 70%, e.g., 75%, 80%, 85%, 90%, 91%, 92%, 93%, 94%, 95%, 96%, 97%, 98%, or 99% identity to the mature polypeptide coding sequence of SEQ ID NO: 3 in WO 2013/148993; and
(iv) a cellobiohydrolase II encoded by a polynucleotide that hybridizes under at least high stringency conditions, e.g., very high stringency conditions, with the mature polypeptide coding sequence of SEQ ID NO: 3 in WO 2013/148993 or the full-length complement thereof.

### Cellulolytic Compositions

As mentioned above the cellulolytic composition may comprise a number of difference polypeptides, such as enzymes.

In an embodiment the cellulolytic composition comprises a Trichoderma reesei cellulolytic composition, further comprising Thermoascus aurantiacus GH61A polypeptide having cellulolytic enhancing activity (WO 2005/074656) and Aspergillus oryzae beta-glucosidase fusion protein (WO 2008/057637).

In another embodiment the cellulolytic composition comprises a Trichoderma reesei cellulolytic composition, further comprising Thermoascus aurantiacus GH61A polypeptide having cellulolytic enhancing activity (SEQ ID NO: 2 in WO 2005/074656) and Aspergillus fumigatus beta-glucosidase (SEQ ID NO: 2 of WO 2005/047499).

In another embodiment the cellulolytic composition comprises a Trichoderma reesei cellulolytic composition, further comprising Penicillium emersonii GH61A polypeptide having cellulolytic enhancing activity disclosed in WO 2011/041397, Aspergillus fumigatus beta-glucosidase (SEQ ID NO: 2 of WO 2005/047499) or a variant thereof with the following substitutions: F100D, S283G, N456E, F512Y.

The enzyme composition of the present invention may be in any form suitable for use, such as, for example, a crude fermentation broth with or without cells removed, a cell lysate with or without cellular debris, a semi-purified or purified enzyme composition, or a host cell, e.g., Trichoderma host cell, as a source of the enzymes.

The enzyme composition may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

In an preferred embodiment the cellulolytic composition comprising a beta-glucosidase having a Relative ED50 loading value of less than 1.00, preferably less than 0.80, such as preferably less than 0.60, such as between 0.1-0.9, such as between 0.2-0.8, such as 0.30-0.70.

In an embodiment cellulolytic enzyme composition is dosed (i.e. during saccharification in step ii) and/or fermentation in step iii) or SSF) from 0.0001-3 mg EP/g DS, preferably 0.0005-2 mg EP/g DS, preferably 0.001-1 mg/g DS, more preferred from 0.005-0.5 mg EP/g DS, even more preferred 0.01-0.1 mg EP/g DS.

Protease Present and/or Added During Liquefaction and/or Saccharification and/or Fermentation

In an embodiment of the invention an optional protease, such as a thermostable protease, may be present and/or added in liquefaction together with an alpha-amylase, such as a thermostable alpha-amylase, and a hemicellulase, preferably xylanase, having a melting point (DSC) above 80° C., and optionally an endoglucanase, a carbohydrate-source generating enzyme, in particular a glucoamylase, optionally a pullulanase, optionally a phospholipase C, and/or optionally a phytase.

In an embodiment of the invention an optional protease may be present and/or added in saccharification step (b), fermentation step (c), simultaneous saccharification and fermentation, or presaccharification prior to step (b) optionally together with an alpha-amylase, a glucoamylase, a cellulolytic composition, and a trehalase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section of WO 2017/112540 (incorporated herein by reference), of which the so-called “AZCL-Casein Assay” is the preferred assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the JTP196 variant (Example 2 from WO 2017/112540) or Protease Pfu (SEQ ID NO: 26 herein) determined by the AZCL-casein assay described in the “Materials & Methods”-section in WO 2017/112540.

There are no limitations on the origin of the thermostable protease used in a process or composition of the invention as long as it fulfills the thermostability properties defined below.

In one embodiment the protease is of fungal origin.

In a preferred embodiment the thermostable protease is a variant of a metallo protease as defined above. In an embodiment the thermostable protease used in a process or composition of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of the mature part of the metallo protease shown in SEQ ID NO: 2 disclosed in WO 2003/048353 or the mature part of SEQ ID NO: 1 in WO 2010/008841 and shown as SEQ ID NO: 27 herein further with mutations selected from below list:
S5*+D79L+S87P+A112P+D142L;
D79L+S87P+A112P+T124V+D142L;
S5*+N26R+D79L+S87P+A112P+D142L;
N26R+T46R+D79L+S87P+A112P+D142L;
T46R+D79L+S87P+T116V+D142L;
D79L+P81R+S87P+A112P+D142L;
A27K+D79L+S87P+A112P+T124V+D142L;
D79L+Y82F+S87P+A112P+T124V+D142L;
D79L+Y82F+S87P+A112P+T124V+D142L;
D79L+S87P+A112P+T124V+A126V+D142L;
D79L+S87P+A112P+D142L;
D79L+Y82F+S87P+A112P+D142L;
S38T+D79L+S87P+A112P+A126V+D142L;
D79L+Y82F+S87P+A112P+A126V+D142L;
A27K+D79L+S87P+A112P+A126V+D142L;
D79L+S87P+N98C+A112P+G135C+D142L;
D79L+S87P+A112P+D142L+T141C+M161C;
S36P+D79L+S87P+A112P+D142L;
A37P+D79L+S87P+A112P+D142L;
S49P+D79L+S87P+A112P+D142L;
S50P+D79L+S87P+A112P+D142L;
D79L+S87P+D104P+A112P+D142L;
D79L+Y82F+S87G+A112P+D142L;
S70V+D79L+Y82F+S87G+Y97W+A112P+D142L;
D79L+Y82F+S87G+Y97W+D104P+A112P+D142L;
S70V+D79L+Y82F+S87G+A112P+D142L;
D79L+Y82F+S87G+D104P+A112P+D142L;
D79L+Y82F+S87G+A112P+A126V+D142L;
Y82F+S87G+S70V+D79L+D104P+A112P+D142L;
Y82F+S87G+D79L+D104P+A112P+A126V+D142L;
A27K+D79L+Y82F+S87G+D104P+A112P+A126V+D142L;
A27K+Y82F+S87G+D104P+A112P+A126V+D142L;
A27K+D79L+Y82F+D104P+A112P+A126V+D142L;
A27K+Y82F+D104P+A112P+A126V+D142L;
A27K+D79L+S87P+A112P+D142L;
D79L+S87P+D142L.

In a preferred embodiment the thermostable protease is a variant of the mature metallo protease disclosed as the mature part of SEQ ID NO: 2 disclosed in WO 2003/048353 or the mature part of SEQ ID NO: 1 in WO 2010/008841 or SEQ ID NO: 27 herein with the following mutations:

### D79L+S87P+A112P+D142L;

### D79L+S87P+D142L; or

### A27K+D79L+Y82F+S87G+D104P+A112P+A126V+D142L.

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 27 herein.

The thermostable protease may also be derived from any bacterium as long as the protease has the thermostability properties defined according to the invention.

In an embodiment the thermostable protease is derived from a strain of the bacterium Pyrococcus, such as a strain of Pyrococcus furiosus (pfu protease).

In an embodiment the protease is one shown as SEQ ID NO: 1 in U.S. Pat. No. 6,358,726-B1 (Takara Shuzo Company) and SEQ ID NO: 26 herein.

In an embodiment the thermostable protease is one disclosed in SEQ ID NO: 26 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 26 herein. The Pyroccus furiosus protease can be purchased from Takara Bio, Japan.

The Pyrococcus furiosus protease is a thermostable protease according to the invention. The commercial product Pyrococcus furiosus protease (Pfu S) was found (see Example 5 of) to have a thermostability of 110% (80° C./70° C.) and 103% (90° C./70° C.) at pH 4.5 determined as described in Example 2 of WO 2017/112540.

In one embodiment a thermostable protease has a thermostability value of more than 20% determined as Relative Activity at 80° C./70° C. determined as described in Example 2.

In an embodiment the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In an embodiment protease has a thermostability of between 20 and 50%, such as between 20 and 40%, such as 20 and 30% determined as Relative Activity at 80° C./70° C.

In an embodiment the protease has a thermostability between 50 and 115%, such as between 50 and 70%, such as between 50 and 60%, such as between 100 and 120%, such as between 105 and 115% determined as Relative Activity at 80° C./70° C.

In an embodiment the protease has a thermostability value of more than 10% determined as Relative Activity at 85° C./70° C. determined as described in Example 2 of WO 2017/112540.

In an embodiment the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In an embodiment the protease has a thermostability of between 10 and 50%, such as between 10 and 30%, such as between 10 and 25% determined as Relative Activity at 85° C./70° C.

In an embodiment the protease has more than 20%, more than 30%, more than 40%, more than 50%, more than 60%, more than 70%, more than 80%, more than 90% determined as Remaining Activity at 80° C.; and/or

In an embodiment the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in Example 2 of WO 2017/112540.

In an embodiment the protease may have a thermostability for above 90, such as above 100 at 85° C. as determined using the Zein-BCA assay as disclosed in Example 3 of WO 2017/112540.

In an embodiment the protease has a thermostability above 60%, such as above 90%, such as above 100%, such as above 110% at 85° C. as determined using the Zein-BCA assay.

In an embodiment protease has a thermostability between 60-120, such as between 70-120%, such as between 80-120%, such as between 90-120%, such as between 100-120%, such as 110-120% at 85° C. as determined using the Zein-BCA assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by the AZCL-casein assay described in the “Materials & Methods”-section of WO 2017/112540.

Additional proteases suitable for use in processes of the invention are shown in SEQ ID Nos: 9-73 (or variants thereof having at least 60%, at least 65%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto) of Table 1 of U.S. Application No. 62/514,636, filed Jun. 2, 2017 (Attorney Docket No. 14480-US-PRO), which is incorporated by reference herein in its entirety.

In various embodiments, the proteases can be expressed with the fermenting organism, e.g., yeast, e.g., a Saccharomyces strain, such as a Saccharomyces cerevisiae strain and processes described herein. In certain embodiments, the proteases are expressed with the fermenting organism, e.g., yeast, e.g., a Saccharomyces strain, such as a Saccharomyces cerevisiae strain, in saccharification, fermentation, simultaneous saccharification and fermentation steps of processes for producing a fermentation product, such as especially ethanol

Trehalases Used in Saccharification and/or Fermentation

According to the invention a trehalase may be present and/or added in saccharification step (b), fermentation step (c), simultaneous saccharification and fermentation (SSF); or presaccharification before step (b) optionally together with an alpha-amylase, a cellulolytic composition, a protease, and any combination thereof.

Trehalases are enzymes which degrade trehalose into its unit monosaccharides (i.e., glucose). According to the invention trehalase may be one single trehalase, or a combination of two of more trehalases of any origin, such as plant, mammalian, or microbial origin, such a bacterial or fungal origin. In a preferred embodiment the trehalase is of mammalian origin, such as porcine trehalase. In another preferred embodiment the trehalase is of fungal origin, preferably of yeast origin. In a preferred embodiment the trehalase is derived from a strain of Saccharomyces, such as a strain of Saccharomyces cervisae.

Trehalases are classified in EC 3.2.1.28 (alpha,alpha-trehalase) and EC. 3.2.1.93 (alpha,alpha-phosphotrehalase). The EC classes are based on recommendations of the Nomenclature Committee of the International Union of Biochemistry and Molecular Biology (IUBMB). Description of EC classes can be found on the internet, e.g., on “http://www.expasy.orq/enzvmer. Trehalases are enzymes that catalyze the following reactions:

### EC 3.2.1.28:

Alpha,alpha-trehalose+H₂O=2 D-glucose;

### EC 3.2.1. 93:

Alpha,alpha-trehalose 6-phosphate+H₂O<=>D-glucose+D-glucose 6-phosphate;

The two enzyme classes are both referred to as “trehalases” in context of the present invention. In a preferred embodiment the trehalase is classified as EC 3.2.1.28. In another embodiment the trehalase is classified as EC 3.2.1.93. In embodiment the trehalase is a neutral trehalase. In another embodiment the trehalase is an acid trehalase.

The trehalase present and/or added during saccharification step (b); fermentation step (c); simultaneous saccharification and fermentation; or presaccharification before step (b), may be derived from any suitable source, e.g., derived from a microorganism or a plant.

Examples of neutral trehalases include, but are not limited to, treahalases from Saccharomyces cerevisiae (Londesborouh et al. (1984) Characterization of two trehalases from baker's yeast” Biochem J 219, 511-518; Mucor roxii (Dewerchin et al (1984),“Trehalase activity and cyclic AMP content during early development of Mucor rouxii spores”, J. Bacteriol. 158, 575-579); Phycomyces blakesleeanus (Thevelein et al (1983), “Glucose-induced trehalase activation and trehalose mobilization during early germination of Phycomyces blakesleeanus spores” J. Gen Microbiol. 129, 719-726); Fusarium oxysporium (Amaral et al (1996), “Comparative study of two trehalase activities from Fusarium oxysporium var Linii” Can. J Microbiol. 41, 1057-1062);

Examples of neutral trehalases include, but are not limited to, trehalases from Saccharomyces cerevisiae (Parvaeh et al. (1996) Purification and biochemical characterization of the ATH1 gene product, vacuolar acid trehalase from Saccharomyces cerevisae” FEBS Lett. 391, 273-278); Neorospora crassa (Hecker et al (1973), “Location of trehalase in the ascospores of Neurospora: Relation to ascospore dormancy and germination”. J. Bacteriol. 115, 592-599); Chaetomium aureum (Sumida et al. (1989), “Purification and some properties of trehalase from Chaetomium aureum MS-27. J. Ferment. Bioeng. 67, 83-86); Aspergillus nidulans (d′Enfert et al. (1997), “Molecular characterization of the Aspergillus nidulans treA gene encoding an acid trehalase required for growth on trehalose. Mol. Microbiol. 24, 203-216); Humicola grisea (Zimmermann et al. (1990).” Purification and properties of an extracellular conidial trehalase from Humicola grisea var. thermoidea“, Biochim. Acta 1036, 41-46); Humicola grisea (Cardello et al. (1994), “A cytosolic trehalase from the thermophilhilic fungus Humicola grisea var. thermoidea, Microbiology UK 140, 1671-1677; Scytalidium thermophilum (Kadowaki et al. (1996), “Characterization of the trehalose system from the thermophilic fungus Scytalidium thermophilum” Biochim. Biophys. Acta 1291, 199-205); and Fusarium oxysporium (Amaral et al (1996), “Comparative study of two trehalase activities from Fusarium oxysporium var Linii” Can. J Microbiol. 41, 1057-1062).

A trehalase is also know from soybean (Aeschbachet et al (1999)” Purification of the trehalase GmTRE1 from soybean nodules and cloning of its cDNA“, Plant Physiol 119, 489-496).

Trehalases are also present in small intestine and kidney of mammals.

In an embodiment, the trehalase is derived from a strain of Talaromyces, such as strain of Talaromyces funiculosus, such as the one shown in SEQ ID NO: 28 herein, or one having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 28 herein, a strain of Talaromyces leycettanus such as the one shown in SEQ ID NO: 29 herein, or one having at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO: 29 herein, or a strain of Talaromyces cellulyticus, such as the one having Accession No: Uniprot:A0A0B8MYG3, or a variant thereof having at least 60%, preferably at least 65%, at least 70%, at least 75%, 80%, at least 85%, at least 90%, at least 91%, at least 92%, at least 93%, at least 94%, at least 95%, at least 96%, at least 97%, at least 98%, at least 99% sequence identity thereto.

In an embodiment, the trehalase is derived from a strain of Myceliophthora, such as a strain of Myceliophthora thermophile, such as one disclosed in WO 2012/027374 (incorporated herein by reference in its entirety) Dyadic) or variants thereof having at least 60%, preferably at least 65%, at least 70%, at least 75%, 80%, at least 85%, at least 90%, at least 91%, at least 92%, at least 93%, at least 94%, at least 95%, at least 96%, at least 97%, at least 98%, at least 99% sequence identity thereto, or from a strain of Myceliophthora sepedonium belonging to Family 37 Glucoside Hydrolases (“GH37″) as defined by the CAZY database (available on the world wide web) having high thermostability and a broad pH range, or variants thereof having at least 60%, preferably at least 65%, at least 70%, at least 75%, 80%, at least 85%, at least 90%, at least 91%, at least 92%, at least 93%, at least 94%, at least 95%, at least 96%, at least 97%, at least 98%, at least 99% sequence identity thereto.

In an embodiment, the trehalase is derived from a strain of Trichoderma, such as a strain of Triochoderma reesei, such as one disclosed in WO 2013/148993 (incorporated herein by reference in its entirety), or a variant thereof having at least 60%, preferably at least 65%, at least 70%, at least 75%, 80%, at least 85%, at least 90%, at least 91%, at least 92%, at least 93%, at least 94%, at least 95%, at least 96%, at least 97%, at least 98%, at least 99% sequence identity thereto.

In an embodiment, the trehalase is derived from a strain of Aspergillus, such as a strain of Aspergillus wentii, such as the one having Accession No: Uniprot:A0A1L9RM22, or a variant thereof having at least 60%, preferably at least 65%, at least 70%, at least 75%, 80%, at least 85%, at least 90%, at least 91%, at least 92%, at least 93%, at least 94%, at least 95%, at least 96%, at least 97%, at least 98%, at least 99% sequence identity thereto.

Commercially available trehalase includes the porcine trehalase available from SIGMA, USA (product #A8778).

The trehalase may be added or present in any effective dosage during fermentation, which includes, but is not limited to, from 1 to 500 Sigma units per liter fermentation medium, preferably 10-100 Sigma units per liter fermentation medium.

### X. Further Aspects of the Invention

In a further aspect of the invention it relates to the use of a peroxidase or peroxidase composition for increasing the growth and/or productivity of yeast.

In a further aspect of the invention it relates to the use of a peroxidase or peroxidase composition for increasing the growth and/or productivity of yeast during yeast propagation.

In a further aspect of the invention it relates to the use of a peroxidase or peroxidase composition for increasing the growth and/or productivity of yeast during ethanol fermentation.

In a further aspect of the invention it relates to the use of a peroxidase or peroxidase composition for increasing the rate at which ethanol is produced within the first 24 hours of fermentation during a biofuel production process.

In a further aspect of the invention it relates to the use of a peroxidase or peroxidase composition for reducing the levels of lactic acid in a biofuel fermentation system.

In a further aspect of the invention it relates to the use of a peroxidase or peroxidase composition for reducing the levels of lactic acid in a fermentation medium.

In a further aspect of the invention it relates to the use of a peroxidase or peroxidase composition for reducing lactic acid titers during the fermentation or simultaneous saccharification and fermentation steps of a biofuel production process.

In a further aspect of the invention it relates to the use of a peroxidase or peroxidase composition for reducing the levels of lactic acid during yeast propagation.

In a further aspect of the invention it relates to the use of a peroxidase or peroxidase composition for reducing lactic acid titers during the fermentation or simultaneous saccharification and fermentation steps of a biofuel production process.

Those skilled in the art will appreciate that the aspects and embodiments described in this section are applicable to any peroxidase, for instance the peroxidases described in section VII herein.

In an embodiment, the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase.

Preferably, the peroxidase is derived from a microorganism, such as a fungal organism, such a yeast or filamentous fungi, or bacteria; or plant.

In a preferred embodiment, the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

The invention is further summarized in the following paragraphs:

1. A process for enhancing yeast growth and/or productivity, the process comprising contacting yeast with an effective amount of a peroxidase.

2. A process for the production of yeast comprising cultivating the yeast of claim 1 under conditions conducive for yeast growth.

3. The process of paragraph 1 or 2, wherein the growth of the yeast is increased from 10% to 50% in comparison to growth of yeast not contacted with the polypeptide.

4. The process of any one of paragraphs 1 to 3, wherein the productivity of the yeast is increased from 10% to 50% in comparison to productivity of yeast not contacted with the polypeptide.

5. A composition comprising yeast produced according to the process of any one of paragraphs 1 to 4 and at least one component selected from a surfactant, an emulsifier, a gum, a swelling agent, an antioxidant, and any combination thereof.

6. The composition according to paragraph 5, which is formulated as a cream yeast, a compressed yeast, a crumbled yeast, or an active dry yeast.

7. A container comprising the composition according to paragraph 5 or 6, wherein the container is optionally selected from a tote, a dosage skid, a package, a sack, or a fermentation vessel.

8. A process for propagating yeast for bioproduct production in a biofuel fermentation system, the process comprising introducing an enzyme composition comprising a peroxidase to a biofuel fermentation system, wherein the fermentation system comprises one or more fermentation vessels, pipes and/or components, and wherein the peroxidase is added at a concentration sufficient to enhance yeast growth and/or productivity in the biofuel fermentation system.

9. The process of any of paragraph 8, wherein at least one of the fermentation vessels is a fermentation tank and the enzyme composition is introduced into the fermentation tank.

10. The process of paragraph 8 or 9, wherein the enzyme composition is introduced into the fermentation tank within the first six hours of fermentation.

11. The process of any one of paragraph 8 to 10, wherein the rate at which ethanol is produced within the first 24 hours of fermentation is increased by from 10% to 50% compared to the amount of ethanol produced within the first 24 hours without the peroxidase.

12. The process of any one of paragraphs 8 to 11, wherein the growth of yeast within the first 24 hours of fermentation is increased by from 10% to 50% compared to the growth of yeast within the first 24 hours of fermentation without the peroxidase.

13. The process of any one of paragraphs 8 to 12, wherein at least one of the fermentation vessels is a yeast propagation tank and the enzyme composition is introduced into the yeast propagation tank.

14. The process of any one of paragraphs 8 to 13, wherein the rate at which ethanol is produced within the first 24 hours of fermentation is increased by from 10% to 50% compared to the amount of ethanol produced within the first 24 hours without the peroxidase.

15. The process of any one of paragraphs 8 to 14, wherein the growth of yeast after 24 hours of propagation is increased by from 10% to 50% in the presence of the peroxidase compared to the growth of yeast over the same period of propagation without the peroxidase.

16. The process of any one of paragraphs 8 to 15, further comprising adding yeast to the propagation tank or to the fermentation vessel.

17. The process of paragraph 16, wherein the yeast is contacted with a peroxidase prior to being added to the propagation tank or the fermentation vessel.

18. The process of any one of paragraphs 8 to 17, wherein the biofuel is ethanol.

19. A process for producing a fermentation product from a starch-containing material, the process comprising:

a) liquefying a starch-containing material in the presence of an alpha-amylase to form a liquefied mash;
b) saccharifying the liquefied mash using a carbohydrate source generating enzyme to produce a fermentable sugar;
c) fermenting the sugar using a fermenting organism under conditions suitable to produce the fermentation product,

wherein a peroxidase is added before or during saccharifying step b) and/or fermenting step c).

20. The process of paragraph 19, wherein steps b) and c) are carried out simultaneously.

21. The process of paragraph 19 or 20, wherein a slurry of the starch containing material is heated to above the gelatinization temperature.

22. The process of any one of paragraphs 19 to 21, wherein the a peroxidase is added during liquefaction.

23. The process of any one of paragraphs 19 to 22, wherein the a peroxidase is added during saccharification, wherein the peroxidase is optionally added within the first 2 hours of saccharification.

24. The process of any one of paragraphs 19 to 23, wherein the a peroxidase is added during fermentation, wherein the peroxidase is optionally added within the first 6 hours fermentation.

25. The process of any one of paragraphs 19 to 24, wherein the fermentation product is an alcohol, preferably ethanol.

26. The process of any one of paragraphs 19 to 25, wherein the fermenting organism is yeast.

27. The process of any one of paragraphs 1 to 26, wherein the yeast belongs to a genus selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera.

28. The process of any one of paragraphs 1 to 27, wherein the yeast is Saccharomyces cerevisiae, Saccharomyces pastorianus (carlsbergiensis), Kluyveromyces lactis, Kluyveromyces fragilis, Fusarium oxysporum, or any combination thereof.

29. The process of any one of paragraphs 1 to 28, wherein the yeast is Saccharomyces cerevisiae.

30. The process of any one of paragraphs 1 to 29, wherein the yeast comprises a heterologous polynucleotide encoding an enzyme selected from an alpha-amylase, a glucoamylase, or a protease.

31. The process of any one of paragraphs 1 to 30, wherein the a peroxidase is added during yeast propagation.

32. The process of paragraph 31, wherein the growth of yeast within the first 24 hours of yeast propagation is increased by from 10% to 50% compared to growth of yeast within the first 24 hours of yeast propagation without the peroxidase.

33. The process of any one of paragraphs 19 to 32, wherein the rate at which ethanol produced within the first 24 hours of fermentation is increased by from 10% to 50% compared to the amount of ethanol produced within the first 24 hours of fermentation without the peroxidase.

34. The process of any one of paragraphs 19 to 33, wherein absolute titers of lactic acid at the end of fermentation are reduced by from 10% to 50% compared to absolute titers of lactic acid at the end of fermentation without the peroxidase.

35. The process of any one of paragraphs 19 to 34, wherein titers of lactic acid within the first 24 hours of fermentation are reduced by from 10% to 50% compared to titers of lactic acid within the first 24 hours of fermentation without the peroxidase.

36. The process of any one of paragraphs 1 to 35, wherein the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase.

37. The process of any one of paragraphs 1 to 36, wherein the peroxidase is derived from a microorganism, such as a fungal organism, such a yeast or filamentous fungi, or bacteria; or plant.

38. The process of any one of paragraphs 1 to 37, wherein the peroxidase is selected from: (i) a peroxidase derived from a strain of Thermoascus, such as strain of Thermoascus aurantiacus, such as the one shown in SEQ ID NO: 1 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1 herein; (ii) a peroxidase derived from a strain of Mycothermus, such as strain of Mycothermus thermophilus, such as the one shown in SEQ ID NO: 2 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 herein; or (iii) a peroxidase derived from a strain of Coprinus, such as strain of Coprinus cinereus, such as the one shown in SEQ ID NO: 3 herein, or one having at least 60%, preferably at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3 herein.

39. Use of a peroxidase according to any one of paragraphs 36 to 39 for propagating yeast.

40. Use of a peroxidase according to any one of paragraphs 36 to 39 increasing the growth and/or productivity of yeast.

41. Use of a peroxidase according to any one of paragraphs 36 to 39 for increasing rate at which ethanol is produced within the first 24 hours of fermentation during a biofuel production process.

42. Use of a peroxidase according to any one of paragraphs 36 to 39 for reducing lactic acid titers during the fermentation or simultaneous saccharification and fermentation steps of a biofuel production process.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. All references are specifically incorporated by reference for that which is described.

The following examples are offered to illustrate certain aspects of the present invention, but not in any way intended to limit the scope of the invention as claimed.

### Materials & Methods

T.a. Catalase: Thermoascus aurantiacus polypeptide having peroxidase activity classified as an E.C. 1.11.1.6 catalase and having the amino acid sequence of SEQ ID NO: 1.

M.t. Catalase: Mycothermus thermophilus polypeptide having peroxidase activity classified as a E.C. 1.11.1.6 catalase and having the amino acid sequence of SEQ ID NO: 2.

C.c. Peroxidase: Coprinus cinereus polypeptide having peroxidase activity classified as a E.C. 1.11.1.7 peroxidase and having the amino acid sequence of SEQ ID NO: 3.

Alpha-Amylase 369 (AA369): Bacillus stearothermophilus alpha-amylase with the mutations: I181*+G182*+N193F+V59A+Q89R+E129V+K177L+R179E+Q254S+M284V (SEQ ID NO: 22 herein) truncated to 491 amino acids.

Glucoamylase SA (GSA): Blend comprising Talaromyces emersonii glucoamylase disclosed as SEQ ID NO: 34 in WO99/28448, Trametes cingulata glucoamylase disclosed as SEQ ID NO: 2 in WO 06/69289, and Rhizomucor pusillus alpha-amylase with Aspergillus niger glucoamylase linker and starch binding domain (SBD) disclosed in SEQ ID NO: 9 herein having the following substitutions G128D+D143N (activity ratio in AGU:AGU:FAU-F is about 20:5:1).

REDSTAR/ETHANOL RED™ (“ER”): Saccharomyces cerevisiae yeast available from Fermentis/Lesaffre, USA.

Protease Pfu: Protease derived from Pyrococcus furiosus shown in SEQ ID NO: 26 herein.

6% YPD Media: Yeast extract, peptone, and glucose (in place of dextrose) were solubilized in deionized water and then sterile filtered; glucose made up 6% of the total solution.

Nutrient Media: Defined nutrient media consisting of complex carbohydrates, trace metals, and ions similar to that of a typical corn mash; used for standardized measurements of yeast performance.

Cytation: Performed using Biotek CYTATION 5, which combines brighfield and phase contrast microscopy. Integrated imaging software was used to develop a method for typical cell enumeration based on cell shape and size.

Clean corn mash: Corn mash was prepared in our laboratories by liquefying ground corn using AA369 and Protease Pfu at 85° C. for 2 hours.

Infected corn mash: MRS media was inoculated with a mixture of isolated bacteria from infected commercial corn ethanol production plants. MRS culture was grown overnight at 32° C. for up to 24 hours. The culture was then introduced to clean corn mash and then incubated for up to 24 hours, and then aliquoted with 20% glycerol and stored at 4° C. Prior to experimentation, an aliquot would be thawed and then weighed into clean corn mash at a rate of 1% w/w.

### EXAMPLES

### Example 1—Peroxidase for Enhancing Yeast Cell Production and Robustness During Ethanol Fermentation

This example demonstrates that the addition of peroxidases in fermentation enhance yeast cell production and robustness early in ethanol fermentation, and in particular significantly increase ethanol production and decrease lactic acid titers within the first 24 hours of fermentation.

### Fermentation Procedure

Infected corn mash, at a 1% w/w infection rate into clean mash, was weighed into a large vessel where 200 ppm urea was added. The pH was adjusted to approximately pH 5.0 and the % dry solids (DS) were adjusted with tap water to either 20% DS or 32% DS. The adjusted mash was then weighed into 15 mL falcon tubes, where the final reaction volume was 5 g. A commercial glucoamylase blend GSA was dosed at 0.6 AGU/g-dry solids for all treatments. Penicillin was dosed at 25 ppm for a single control treatment. T.a. Catalase or C.c. Peroxidase were dosed at 10, 50, 100, and 200 ppm. Additional tap water was added to normalize treatment volumes. Red Star or ER (activated dried yeast) was rehydrated in tap water at 32° C. for approximately 30 minutes, and then pitched at 0.25 g/L. All samples were capped with a lid with a small hole in the top for CO₂ release. Each sample was then vortexed for approximately 15 seconds prior to incubation at 32° C. for either ˜24 hours (for 20% DS samples) or ˜60 hours (for 32% DS samples). Treatments were run in triplicate. Compounds of interest were measured via HPLC using an ion-exchange H-column. Standards for measurement were: maltotriose (DP3), maltose (DP2), glucose, fructose, arabinose, lactic acid, glycerol, acetic acid, and ethanol. Titers were reported in g/L. These conditions were used to carry out three separate studies, outlined in the results below.

### Results

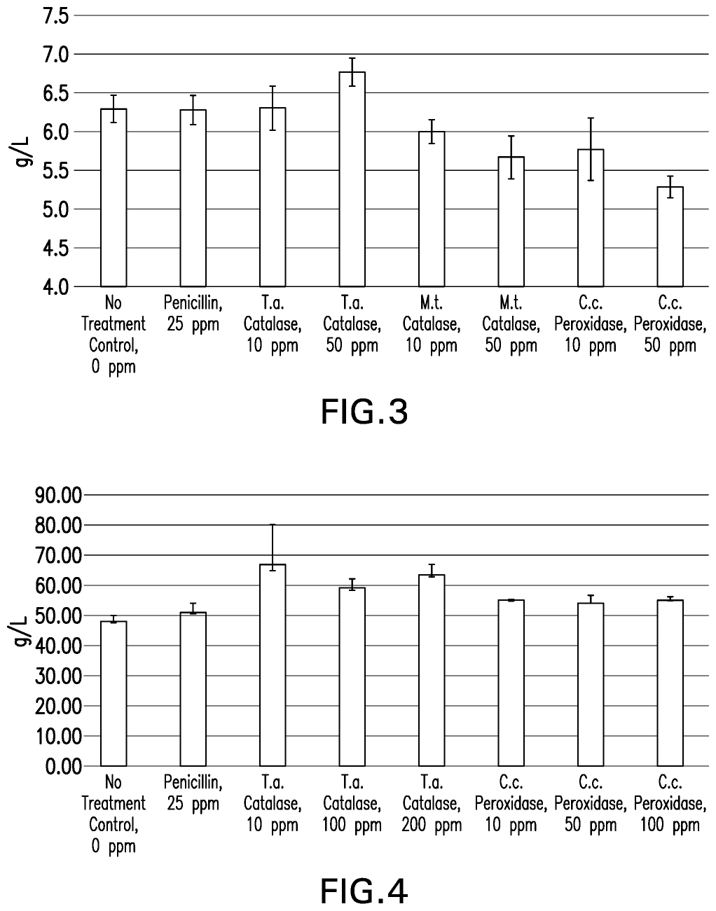

In a first study, the addition of peroxidases during fermentation increased ethanol titers significantly compared to the control lacking peroxidase and penicillin control (FIG. 2). Additionally, three of the treatments showed decreased lactic acid titers compared to the controls (FIG. 3).

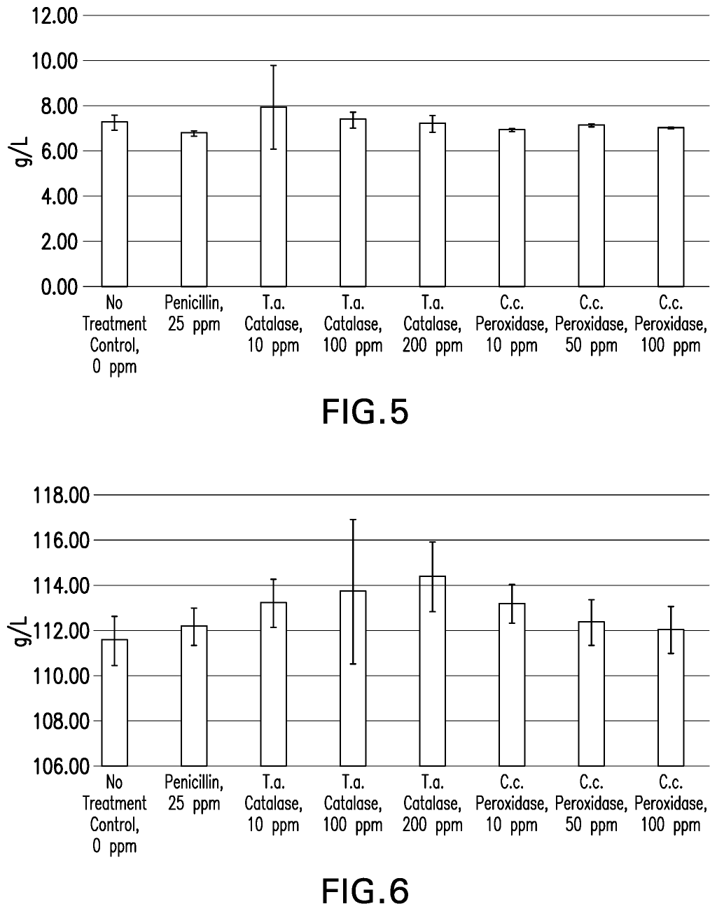

In a second study, the addition of peroxidases during fermentation increased ethanol titers significantly compared to the control lacking peroxidases and penicillin control (FIG. 4). In this case, lactic acid titers were not shown to decrease, but rather shown to be nearly equivalent compared to the controls (FIG. 5).

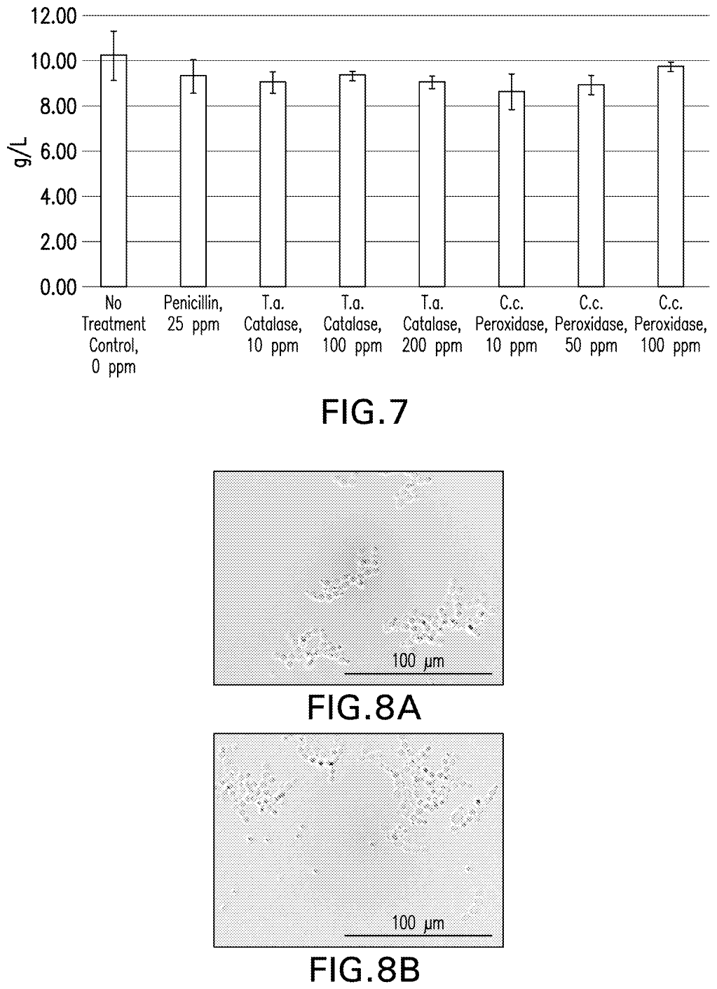

In a third study, the addition of peroxidases during fermentation increased ethanol titers significantly compared to the control lacking peroxidase and penicillin control (FIG. 6). However, C.c. peroxidase did not seem to be as ethanol tolerant as T.a. catalase. As seen in the first study, lactic acid titers decreased compared to the controls (FIG. 7).

### Example 2—Peroxidase for Yeast Cell Growth

This example demonstrates that peroxidases enhance yeast cell growth, and can be used for propagating yeast (e.g., for production of yeast on a commercial scale, for ethanol fermentation, etc.).

### 50 mL Propagation Procedure

Ethanol Red activated dried yeast (ADY) was rehydrated in tap water at 32° C. for approximately 30 minutes. 50 mL YPD media was sterile aliquoted into a baffled sterile 125 mL flask. One loop full of rehydrated yeast (approximately 10 uL) was inoculated into the sterile media. T.a. Catalase was then dosed at 5, 25, 50, and 200 uL of product. A no enzyme treatment was used as a control. Additional sterile water was added as a liquid balance. Treatments were incubated at 32° C. for approximately 1 hour with 100 rpm orbital shaking. Measurement of yeast cells was performed by examining the cells on a Cytation, which using bright field microscopy techniques and cell counting software. The Cytation preparation consists of placing 20 uL of dilute sample into the well of a black 384-well plate with a clear bottom. 20 images per well are taken, then counts are averaged between all images. Samples were done in quadruplicate.

### 14 L Propagation Procedure

Yeast (S. cerevisiae) was initially propagated in nutrient media to allow cells to get to a certain density prior to larger scale propagation. 2 L scale propagations were performed in liquid nutrient media for up to 24 hours at 30° C. with agitation. A portion of the 2 L propagation was used for inoculating 14 L reactors for yeast cell production. 0.5 mL/L of concentrated liquid T.a. Catalase product or 3 mL/L of concentrated M.t. Catalase product was introduced at 14 L scale prior to yeast inoculation. 14 L reactions were performed in liquid nutrient media, titrated over time, at 30° C. to 35° C. with agitation for up to 50 hours.

### Results

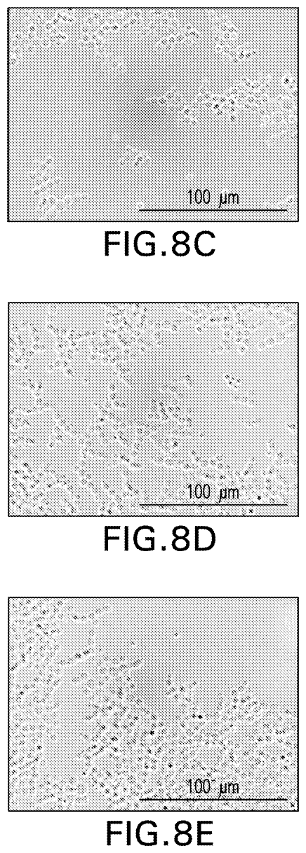

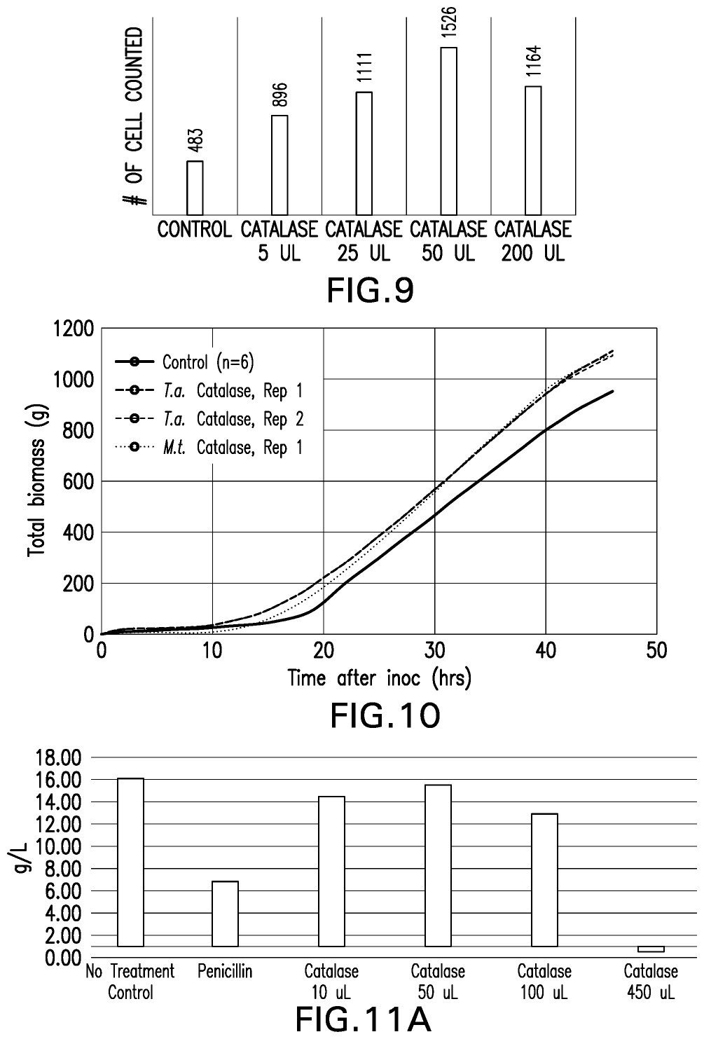

Cytation images showing yeast cell growth in sterile nutrient medium are shown in the figure below (FIG. 8A, FIG. 8B, FIG. 8C, FIG. 8D, and FIG. 8E). As the titration of T.a. Catalase product increases, so does the yeast cell count, i.e. yeast cell biomass generation. Enumeration was performed using software and bright field microscopy techniques. Large population densities have the potential to be undercounted as cells tend to clump together, shown in FIG. 8E and FIG. 9.

In 14 L propagation, a 10% increase in yeast cell biomass was reported at the end of the reaction compared to the control (FIG. 10). This indicates that the addition of either T.a. Catalase or M.t. Catalase can increase yeast cell growth using equal nutrient inputs.

### Example 3—Peroxidases for Yeast Cell Production and Robustness During Propagation

This example demonstrates that peroxidases enhance yeast growth and/or productivity, for example, yeast propagated in the presence of peroxidase consumed more glucose and produced higher ethanol titers within the first 6 hours of propagation. Unexpectedly, when the propagated yeast were transferred into fermentation and yeast were challenged with infection, the peroxidase treated yeast were able to outcompete the infection more productively as measured by reduced lactic acid titers.

### Propagation Procedure

Clean mash was diluted to 20% dried solids (DS) and then 1000 ppm urea and commercial glucoamylase blend GSA were added. The substrate was then weighed into 125 mL baffled shake flasks. Concentrated T.a. Catalase product was dosed at 10 uL up to 450 uL into treatments. Final working volume was 50 g for all treatments. Penicillin and No Treatment were used as controls. Ethanol Red or Red Star activated dry yeast was rehydrated, and then inoculated at equivalent cell densities for all treatments. The samples were incubated at 32° C. for approximately 6 hours with agitation. HPLC measurements were taken and analyzed for soluble carbohydrates and organic acids.

### Fermentation Procedure

Infected corn mash, at a 1% w/w infection rate into clean mash, was weighed into a large vessel where 1000 ppm urea was added. The pH was adjusted to approximately pH 5.0 and the % dry solids (DS) were adjusted with tap water to 32% DS. The adjusted mash was then weighed into Ankom jars. Commercial gluco-amylase, GSA, was dosed at a commercially relevant, equivalent amount for all treatments. No additional catalase enzyme was used during fermentation. Propagation treatments were transferred into fermentation treatments at 5% of the working fermentation volume, where the total working volume was 50 g. Fermentation treatments were run in triplicate. Ankom pressure monitors were used to cap the jars, and gas release was recorded throughout fermentation, reported in psi. Compounds of interest were measured via HPLC using an ion-exchange H-column. Standards for measurement were: maltotriose (DP3), maltose (DP2), glucose, fructose, arabinose, lactic acid, glycerol, acetic acid, and ethanol. Titers were reported in g/L.

### Results

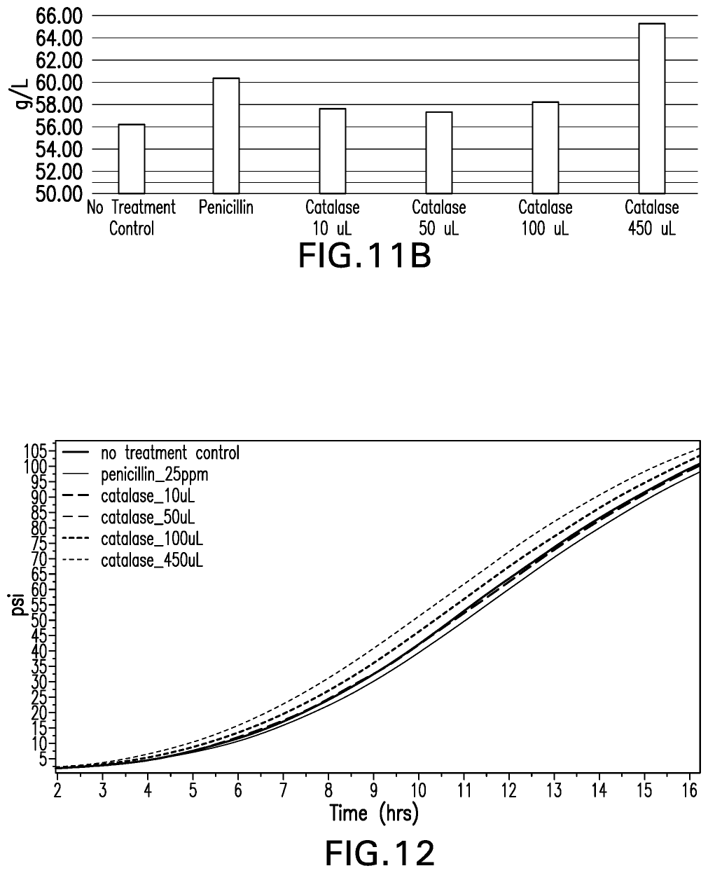

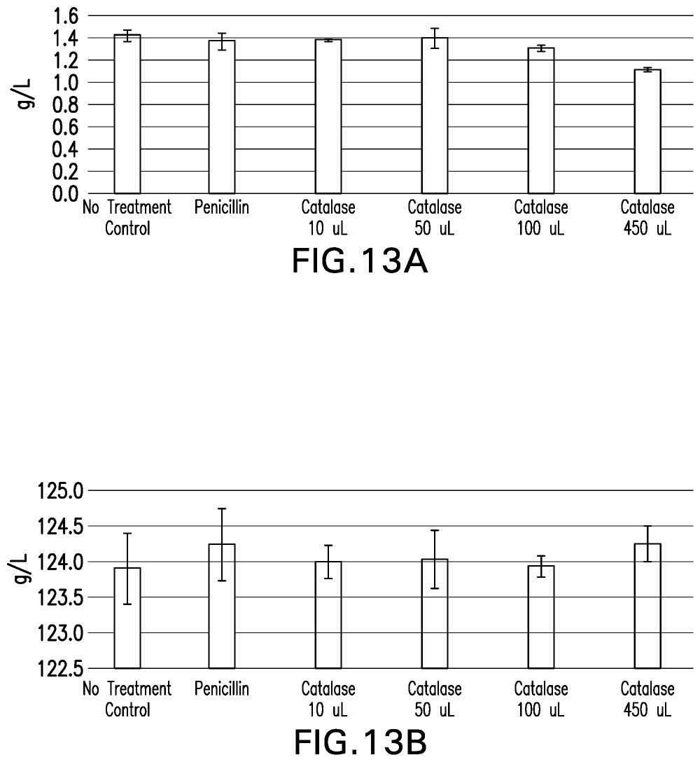

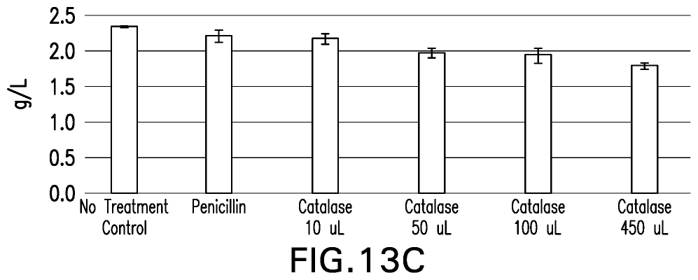

Yeast that were propagated in the presence of catalase consumed more glucose (FIG. 11A) and produced higher titers of ethanol during propagation (FIG. 11B). When the propagations were transferred into fermentation, the treatments with catalase produced off-gas at a faster rate than no treatment or penicillin treatment controls (FIG. 12). As a result, when the yeast was challenged with an infected system, the yeast treated with catalase were able to overcome and outcompete it more productively as measured by lowered lactic acid titers (FIG. 13A). Although at the time the fermentation was measured, approximately 60 hours, the ethanol titers were fairly flat across all treatments (FIG. 13B). However, DP2 titers decreased as catalase dose increased (FIG. 13C).

1. A process for enhancing yeast growth and/or productivity, the process comprising contacting yeast with an effective amount of a peroxidase.

2. A process for the production of yeast comprising cultivating the yeast of claim 1 under conditions conducive for yeast growth.

3. The process of claim 1 or 2, wherein the growth of the yeast is increased from 10% to 50% in comparison to growth of yeast not contacted with the polypeptide.

4. The process of claim 1, wherein the productivity of the yeast is increased from 10% to 50% in comparison to productivity of yeast not contacted with the polypeptide.

5. A composition comprising yeast produced according to the process of claim 1 and at least one component selected from a surfactant, an emulsifier, a gum, a swelling agent, an antioxidant, and any combination thereof.

6. The composition according to claim 5, which is formulated as a cream yeast, a compressed yeast, a crumbled yeast, or an active dry yeast.

7. A container comprising the composition according to claim 5, wherein the container is optionally selected from a tote, a dosage skid, a package, a sack, or a fermentation vessel.

8. A process for propagating yeast for bioproduct production in a biofuel fermentation system, the process comprising introducing an enzyme composition comprising a peroxidase to a biofuel fermentation system, wherein the fermentation system comprises one or more fermentation vessels, pipes and/or components, and wherein the peroxidase is added at a concentration sufficient to enhance yeast growth and/or productivity in the biofuel fermentation system.

9. A process for producing a fermentation product from a starch-containing material, the process comprising:
a) liquefying a starch-containing material in the presence of an alpha-amylase to form a liquefied mash;
b) saccharifying the liquefied mash using a carbohydrate source generating enzyme to produce a fermentable sugar;
c) fermenting the sugar using a fermenting organism under conditions suitable to produce the fermentation product,
wherein a peroxidase is added before or during saccharifying step b) and/or fermenting step c).

10. The process of claim 9, wherein a peroxidase is added during yeast propagation.

11. The process of claim 1, wherein the peroxidase is a peroxidase or peroxide-decomposing enzymes selected from: E.C. 1.11.1.1 NADH peroxidase; E.C. 1.11.1.2 NADPH peroxidase; E.C. 1.11.1.3 fatty-acid peroxidase; E.C. 1.11.1.5 cytochrome-c peroxidase; E.C. 1.11.1.5; E.C. 1.11.1.6 catalase; E.C. 1.11.1.7 peroxidase; E.C. 1.11.1.8 iodide peroxidase; E.C. 1.11.1.9 glutathione peroxidase; E.C. 1.11.1.10 chloride peroxidase; E.C. 1.11.1.11 L-ascorbate peroxidase; E.C. 1.11.1.12 Phospholipid-hydroperoxide glutathione peroxidase; E.C. 1.11.1.13 manganese peroxidase; E.C. 1.11.1.14 lignin peroxidase; E.C. 1.11.1.15 peroxiredoxin; E.C. 1.11.1.16 versatile peroxidase; E.C. 1.11.1.B2 chloride peroxidase; E.C. 1.11.1.B6 iodide peroxidase (vanadium-containing); E.C. 1.11.1.B7 bromide peroxidase; E.C. 1.11.1.B8 iodide peroxidase.

12. The process of claim 1, wherein the peroxidase is derived from a microorganism.

13. (canceled)

14. (canceled)

15. (canceled)

16. The process of claim 1, wherein the peroxidase is derived from a strain of Thermoascus.

17. The process of claim 1, wherein the peroxidase is derived from a strain of Thermoascus aurantiacus.

18. The process of claim 1, wherein the peroxidase has an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence set forth in SEQ ID NO: 1.

19. The process of claim 1, wherein the peroxidase is derived from a strain of Mycothermus.

20. The process of claim 1, wherein the peroxidase is derived from a strain of Mycothermus thermophilus.

21. The process of claim 1, wherein the peroxidase has an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence set forth in SEQ ID NO: 2.

22. The process of claim 1, wherein the peroxidase is derived from a strain of Coprinus.

23. The process of claim 1, wherein the peroxidase is derived from a strain of Coprinus cinereus.

24. The process of claim 1, wherein the peroxidase has an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence set forth in SEQ ID NO: 3.
